(19) United States
(12) Patent Application Publication
(10) Pub. No.: US 2003/0134806 A1
(43) Pub. Date: Jul. 17, 2003

(54) Method and reagent for the inhibition of grid

(76) Inventors: Thale Jarvis, Boulder, CO (US); Ira Von Carlowitz, Boulder, CO (US); James McSwiggen, Boulder, CO (US); Paul Andrew Hamblin, Stevenage (GB); Jonathon Henry Ellis, Stevenage (GB)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/792,818

(22) Filed: Feb. 23, 2001

Related U.S. Application Data
(60) Provisional application No. 60/181,594, filed on Feb. 10, 2000.

(51) Int. Cl. A61K 48/00; C07H 21/04

(52) U.S. Cl. 514/44; 536/23.2

(57) ABSTRACT

The present invention relates to nucleic acid molecules, including antisense and enzymatic nucleic acid molecules, such as hammerhead ribozymes, DNAzymes, and antisense, which modulate the expression of the GRID (Grb2-related with Insert Domain) gene.

This invention claims priority from Jarvis et al., U.S. Ser. No. 60/181,594, filed Feb. 24, 2000, entitled “METHOD AND REAGENT FOR THE INHIBITION OF GRID”. This application is hereby incorporated by reference herein in its entirety including the drawings.

### BACKGROUND OF THE INVENTION

The present invention concerns compounds, compositions, and methods for the study, diagnosis, and treatment of conditions and diseases related to the expression of the T-cell co-stimulatory adapter protein GRID (Grb2-related with Insert Domain).

The following is a brief description of the current understanding of GRID. The discussion is not meant to be complete and is provided only for understanding the invention that follows. The summary is not an admission that any of the work described below is prior art to the claimed invention.

One of the emerging paradigms for signal transduction in lymphocytes is that receptors and other signaling molecules do not operate in isolation, but through the recruitment of a complex of other proteins (Pawson and Scott, 1997; Science, 278, 2075; Rudd, 1999, Cell, 96, 5). These other proteins serve to amplify and diversify the signal into a number of biochemical cascades. The archetypal adapter protein is Grb2, which serves to regulate downstream pathways such as Ras activation and Ca2+ mobilization (Lowenstein et al., 1992, Cell, 70, 431), and is ultimately responsible for modulating gene expression required for proliferation and differentiation. Grb2 is recruited to LAT and SLP-76 which are downstream targets in the signaling cascade initiated by ligation of the T-cell receptor by MHC-antigen. These functions are mediated by specialized domains which bind specific motifs and include the phosphotyrosine binding SH2 (Src homology) domain and SH3 domain which are associated with proline-rich PXXP motifs. Grb2, whose sole function appears to be the formation of bridges between other proteins, is entirely comprised of such domains having an SH3-SH2-SH3 structure (Peterson et al., 1998, Curr. Opin. Immunol., 10, 337; Koretzky, 1997, Immunol Today, 18, 401).

A novel member of the Grb2 family of adapter proteins termed GRID (Grb2-related with Insert Domain) has recently been identified (Asada et al, 1999, J. Exp. Med., 189, 1383; Liu et al., 1999, Curr. Biol., 9, 67; Liu et al., 1998, Oncogene, 17, 3073; Law et al., 1999, J. Exp. Med., 189, 1243; Qiu et al., 1998, Biochem. Biophys. Res. Commun., 253, 443; Bourette et al., 1998, Embo. J, 17, 7273). GRID is recruited to the T cell co-stimulatory receptor CD28 upon activation of this receptor by cross-linking antibodies. Although GRID shares significant similarity at the protein level with Grb2, possessing an SH3-SH2-SH3 domain structure, GRID also contains a unique proline-glutamine rich domain situated between the SH2 and C-terminal SH3 domain. The association of GRID with activated CD28 is absolutely dependent upon the integrity of the SH2 domain and phosphorylation of residue Y173 in the cytoplasmic tail of CD28. Although GRID has been shown to associate with other T cell signaling proteins including SLP-76 and LAT (Asada et al., supra; Liu et al., supra; Law et al., supra), it's role in T cell signaling pathways is not well defined.

Tari et al., 1999, Oncogene, 18(6), 1325-1332, describe the antisense inhibition of Grb2 in breast cancer cells in order to investigate the role of Grb2 in the proliferation of breast cancer cells. The resulting Grb2 inhibition led to MAP kinase inactivation in EGFR but not in ErbB2 expressing breast cancer cells.

Tari et al., 1998, J. Liposome Res., 8(2), 251-264, describe P-ethoxy antisense oligonucleotides targeting Bcr-Ab1, Grb2, Crk1, and Bc1-2 mRNA. Delivery of these antisense oligonucleotides via liposome transfection results in the inhibition of corresponding proteins, thereby inducing growth inhibition in leukemia and lymphoma cell lines.

Lopez-Berestein et al., 1998, International PCT publication No. WO 98/01547, describe inhibition of chronic myelogenous leukemic cell growth by liposomal-antisense oligodeoxynucleotides targeting Grb2 and Crk1.

Tari et al., 1997, Biochem. Biophys. Res. Commun., 235(2), 383-388, describe the antisense-based inhibition of Grb2 and Crk1 proteins results in growth inhbition of Philadelphia chromosome positive leukemic cells.

### SUMMARY OF THE INVENTION

The invention features novel nucleic acid-based techniques [e.g., enzymatic nucleic acid molecules (for example, ribozymes or DNAzymes), antisense nucleic acids, 2-5A antisense chimeras, triplex DNA, antisense nucleic acids containing RNA cleaving chemical groups] and methods for their use to modulate the expression of GRID (Grb2-related with Insert Domain).

The description below of the various aspects and embodiments is provided with reference to the exemplary gene GRID. However, the various aspects and embodiments are also directed to other genes which express GRID-like adapter proteins involved in T-cell co-activation. Those additional genes can be analyzed for target sites using the methods described for GRID. Thus, the inhibition and the effects of such inhibition of the other genes can be performed as described herein.

In a preferred embodiment, the invention features the use of one or more of the nucleic acid-based techniques independently or in combination to inhibit the expression of the genes encoding GRID. For example, the nucleic acid-based techniques of the present invention can be used to inhibit the expression of GRID gene sequences found at GenBank Accession NOS. AJ011736, NM_{—}004810, Y18051, AF121002, AF042380, AF129476, AF090456).

In another preferred embodiment, the invention features the use of an enzymatic nucleic acid molecule, preferably in the hammerhead, NCH (Inozyme), G-cleaver, amberzyme, zinzyme and/or DNAzyme motif, to inhibit the expression of GRID gene.

By “inhibit” it is meant that the activity of GRID or level of GRID RNAs or equivalent RNAs encoding one or more protein subunits of GRID or GRID-like proteins is reduced below that observed in the absence of the nucleic acid molecules of the invention. In one embodiment, the inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence of, for example, an oligonucleotide with scrambled sequence or with mismatches. In another embodiment, inhibition of GRID or GRID-like genes with the nucleic acid molecule of the instant invention is greater than in the presence of the nucleic acid molecule than in its absence.

By “enzymatic nucleic acid molecule” it is meant a nucleic acid molecule which has complementarity in a substrate-binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and may comprise modified or unmodified nucleotides or non-nucleotides or various mixtures and combinations thereof.

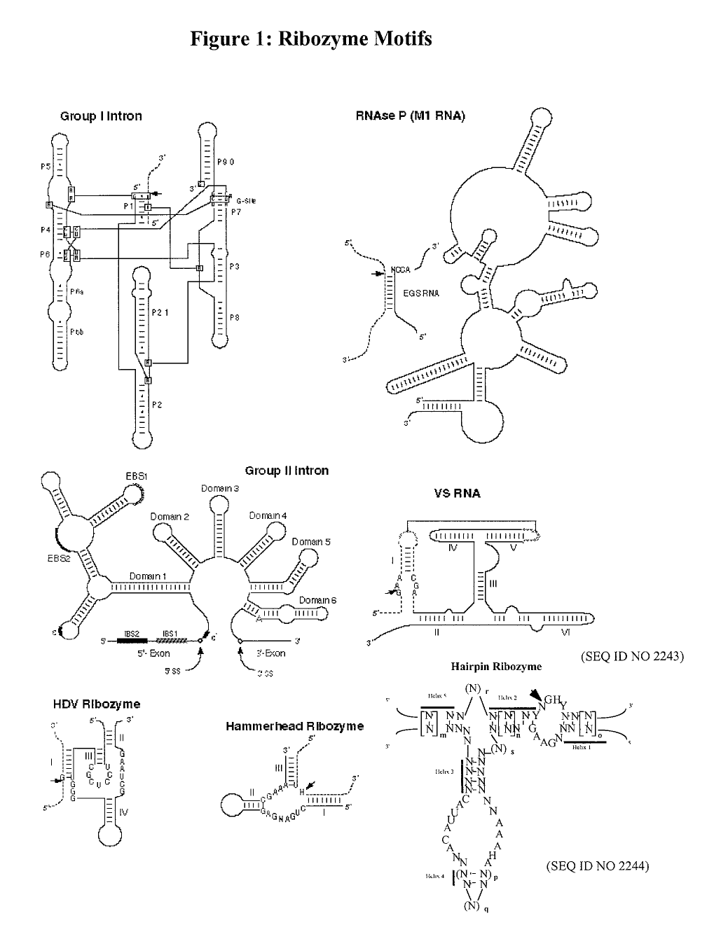

By “enzymatic portion” or “catalytic domain” is meant that portion or region of the enzymatic nucleic acid molecule essential for cleavage of a nucleic acid substrate (for example, see FIGS. 1-5).

By “substrate binding arm” or “substrate binding domain” is meant that portion or region of a enzymatic nucleic acid which is able to interact, for example, via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-5. That is, these arms contain sequences within an enzymatic nucleic acid which are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) are preferably greater than or equal to four nucleotides and of sufficient length to stably interact with the target RNA. Preferably, the binding arm(s) are 12-100 nucleotides in length. More preferably, the binding arms are 14-24 nucleotides in length (see, for example, Werner and Uhlenbeck, supra; Hamman et al., supra; Hampel et al., EP0360257; Berzal-Herrance et al., 1993, EMBOJ, 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

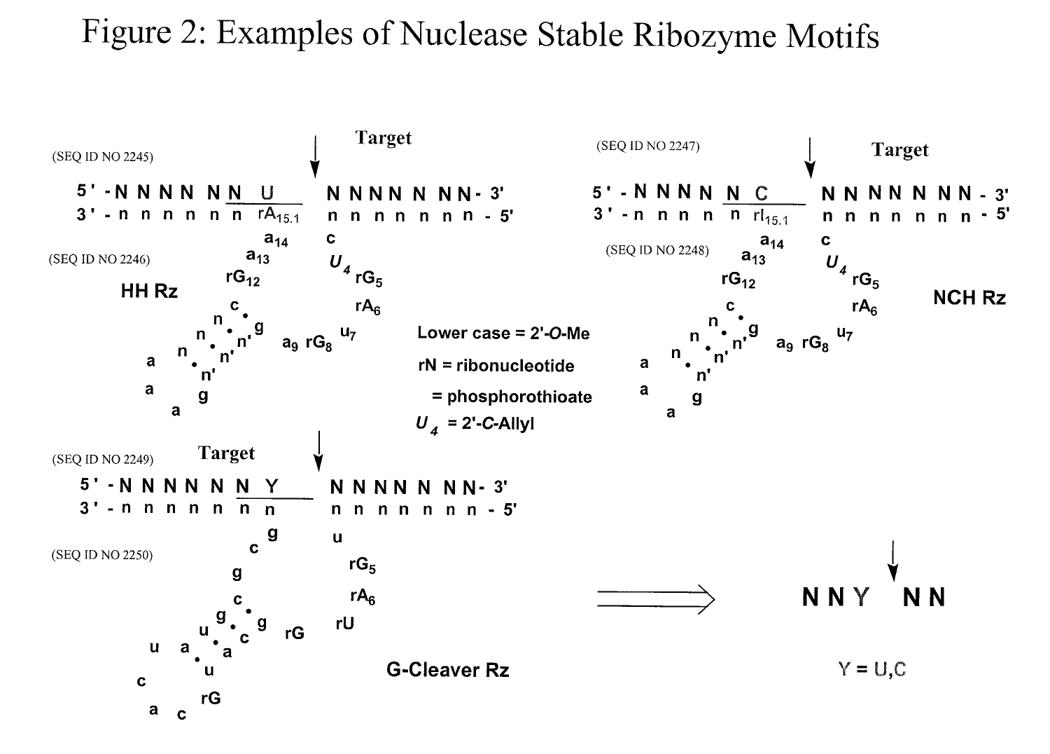

By “Inozyme” or “NCH” motif is meant, an enzymatic nucleic acid molecule comprising a motif as is generally described as NCH Rz in FIG. 2. Inozymes possess endonuclease activity to cleave RNA substrates having a cleavage triplet NCH/, where N is a nucleotide, C is cytidine and H is adenosine, uridine or cytidine, and / represents the cleavage site. H is used interchangeably with X. Inozymes can also possess endonuclease activity to cleave RNA substrates having a cleavage triplet NCN/, where N is a nucleotide, C is cytidine, and / represents the cleavage site. “I” in FIG. 2 represents an Inosine nucleotide, preferably a ribo-Inosine or xylo-Inosine nucleoside.

By “G-cleaver” motif is meant, an enzymatic nucleic acid molecule comprising a motif as is generally described as G-cleaver in FIG. 2. G-cleavers possess endonuclease activity to cleave RNA substrates having a cleavage triplet NYN/, where N is a nucleotide, Y is uridine or cytidine and / represents the cleavage site. G-cleavers may be chemically modified as is generally shown in FIG. 2.

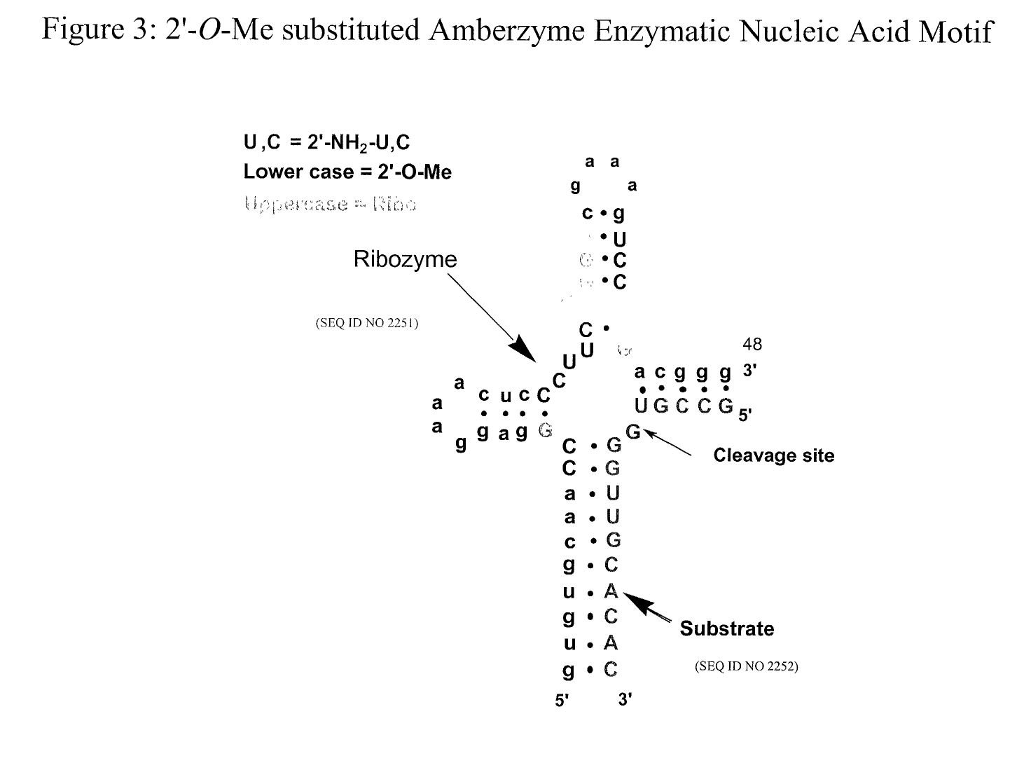

By “amberzyme” motif is meant, an enzymatic nucleic acid molecule comprising a motif as is generally described in FIG. 3. Amberzymes possess endonuclease activity to cleave RNA substrates having a cleavage triplet NG/N, where N is a nucleotide, G is guanosine, and / represents the cleavage site. Amberzymes can be chemically modified to increase nuclease stability through substitutions as are generally shown in FIG. 3. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

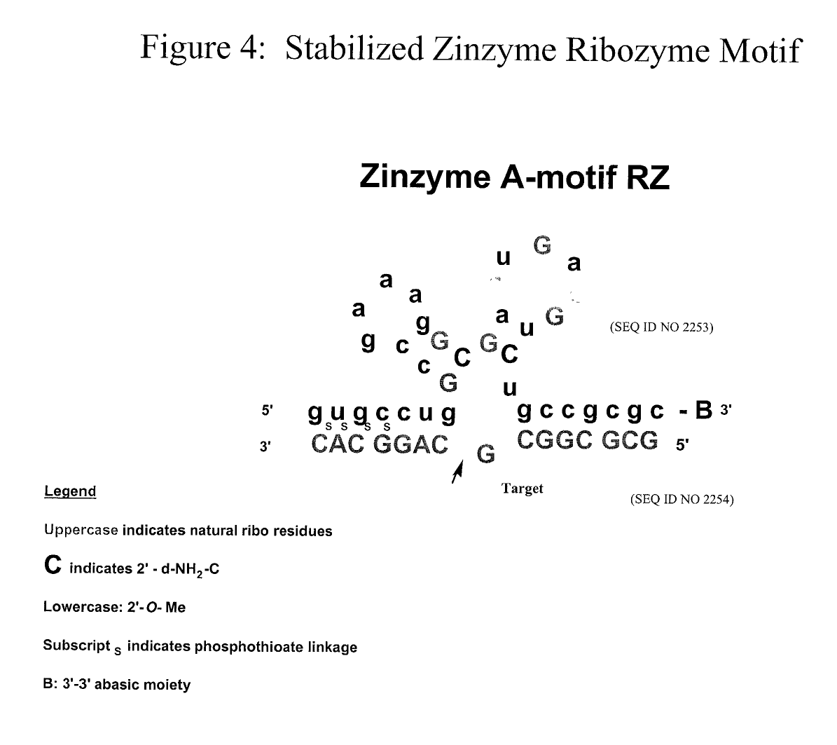

By “zinzyme” motif is meant, an enzymatic nucleic acid molecule comprising a motif as is generally described in FIG. 4. Zinzymes possess endonuclease activity to cleave RNA substrates having a cleavage triplet including but not limited to YG/Y, where Y is uridine or cytidine, and G is guanosine and / represents the cleavage site. Zinzymes can be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

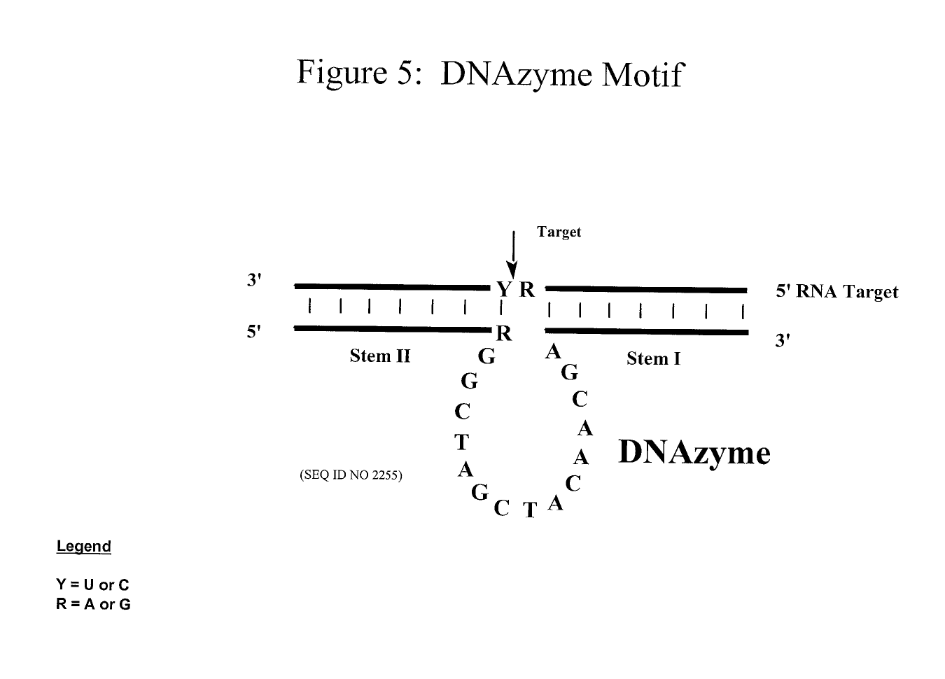

By ‘DNAzyme’ is meant, an enzymatic nucleic acid molecule that does not require the presence of a 2′-OH group for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. Additional DNAzyme motifs can be selected for using techniques similar to those described in these references, and hence, are within the scope of the present invention.

By “sufficient length” is meant an oligonucleotide of greater than or equal to 3 nucleotides that is of a length great enough to provide the intended function under the expected condition.

For example, for binding arms of enzymatic nucleic acid “sufficient length” means that the binding arm sequence is long enough to provide stable binding to a target site under the expected binding conditions. Preferably, the binding arms are not so long as to prevent useful turnover.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” RNA to GRID is meant to include those naturally occurring RNA molecules having homology (partial or complete) to GRID proteins or encoding for proteins with similar function as GRID in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et cal., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more preferably, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target RNA in a sequence- specific manner and activate a cellular 2-5A-dependent ribonuclease which, in turn, cleaves the target RNA (Torrence et al., 1993 Proc. Natl Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “gene” it is meant a nucleic acid that encodes RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSHSymp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “decoy RNA” is meant a RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art.

Several varieties of naturally occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in GRID-specific RNAs represent a novel therapeutic approach to treat a variety of pathologic indications, including but not limited to tissue/graft rejection and leukemia.

In one of the preferred embodiments of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183. Examples of hairpin motifs are described by Hampel et al., EP0360257, Hampel and Tritz, 1989 Biochemistry 28, 4929, Feldstein et al., 1989, Gene 82, 53, Haseloff and Gerlach, 1989, Gene, 82, 43, Hampel et al., 1990 Nucleic Acids Res. 18, 299; and Chowrira & McSwiggen, U.S. Pat. No. 5,631,359. The hepatitis delta virus motif is described by Perrotta and Been, 1992 Biochemistry 31, 16. The RNase P motif is described by Guerrier-Takada et al., 1983 Cell 35, 849; Forster and Altman, 1990, Science 249, 783; and Li and Altman, 1996, Nucleic Acids Res. 24, 835. The Neurospora VS RNA ribozyme motif is described by Collins (Saville and Collins, 1990 Cell, 61, 685-696; Saville and Collins, 1991 Proc. Natl. Acad. Sci. USA 88, 8826-8830; Collins and Olive, 1993 Biochemistry 32, 2795-2799; and Guo and Collins, 1995, EMBO. J. 14, 363). Group II introns are described by Griffin et al., 1995, Chem. Biol. 2, 761; Michels and Pyle, 1995, Biochemistry 34, 2965; and Pyle et al., International PCT Publication No. WO 96/22689. The Group I intron is described by Cech et al., U.S. Pat. No. 4,987,071. DNAzymes are described by Usman et al., International PCT Publication No. WO 95/11304; Chartrand et al., 1995, NAR 23, 4092; Breaker et al., 1995, Chem. Bio. 2, 655; and Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs include the Aptazyme (Breaker et al., WO 98/43993), Amberzyme (Class I motif; FIG. 3; Beigelman et al., International PCT publication No. WO 99/55857) and Zinzyme (Beigelman et al., International PCT publication No. WO 99/55857), all these references are incorporated by reference herein in their totalities, including drawings and can also be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule of the instant invention can be between 13 and 100 nucleotides in length. Exemplary enzymatic nucleic acid molecules of the invention are shown in Tables III-VIII and X. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096 and Cairns et al., 2000, Antisense & Nucleic Acid Drug Dev., 10, 323-332). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for the nucleic acid molecule to be of length and conformation sufficient and suitable for the nucleic acid molecule to catalyze a reaction contemplated herein. The length of the nucleic acid molecules of the instant invention are not limiting within the general limits stated.

Preferably, a nucleic acid molecule that down regulates the replication of GRID or GRID-like gene comprises between 12 and 100 bases complementary to a GRID or GRID-like RNA. Even more preferably, a nucleic acid molecule that down regulates the replication of GRID or GRID-like gene comprises between 14 and 24 bases complementary to a GRID or GRID-like RNA.

In a preferred embodiment, the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding GRID or GRID-like proteins such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to target cells.

In a preferred embodiment, the invention features the use of nucleic acid-based inhibitors of the invention to specifically target genes that share homology with the GRID gene. For example, the invention describes the use of nucleic acid-based inhibitors to target the Grb2 (GenBank accession No. NM_{—}002086) and GRAP (GenBank accession No. NM_{—}006613) genes.

As used in herein “cell” is used in its usual biological sense and does not refer to an entire multicellular organism. The cell can be present in an organism which includes humans but is preferably a non-human multicellular organism, e.g., birds, plants and mammals such as cows, sheep, apes, monkeys, swine, dogs, and cats. The cell can be prokaryotic (e.g., bacterial cell) or eukaryotic (e.g., mammalian or plant cell).

By “GRID proteins” is meant, a protein or a mutant protein derivative thereof, comprising an adapter-protein type of association to the activated CD28 co-stimulatory receptor, and to other signaling proteins including but not limited to SLP-76 and LAT.

By “highly conserved sequence region” is meant a nucleotide sequence of one or more regions in a target gene that does not vary significantly from one generation to the other or from one biological system to the other.

The nucleic acid-based inhibitors of GRID expression are useful for the prevention and/or treatment of diseases and conditions that are related to or will respond to the levels of GRID in a cell or tissue, alone or in combination with other therapies. For example, the nucleic acid-based inhibitors of GRID expressions are useful for the prevention and/or treatment of tissue/graft rejection and cancer, such as leukemia, among other conditions.

By “related” is meant that the reduction of GRID expression (specifically GRID gene) RNA levels and thus reduction in the level of the respective protein will relieve, to some extent, the symptoms of the disease or condition.

In a preferred embodiment, the invention features the use of nucleic acid-based inhibitors of the invention to specifically target regions of GRID gene that are not homologous to Grb2 gene. Specifically, the invention describes the use of nucleic acid-based inhibitors to target sequences that are unique to GRID gene.

The nucleic acid-based inhibitors of the invention are added directly, or can be complexed with cationic lipids, packaged within liposomes, or otherwise delivered to target cells or tissues using well-known methods described herein and generally known in the art. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences, which are complementary to the substrate sequences in Tables III to X. Examples of such enzymatic nucleic acid molecules also are shown in Tables III to VIII and X. Examples of such enzymatic nucleic acid molecules consist essentially of sequences defined in these Tables.

In yet another embodiment, the invention features antisense nucleic acid molecules and 2-5A chimera including sequences complementary to the substrate sequences shown in Tables III to X. Such nucleic acid molecules can include sequences as shown for the binding arms of the enzymatic nucleic acid molecules in Tables III to VIII and X and sequences shown as GeneBloc™ sequences in Table X. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples and binding arms able to bind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Tables III and IV can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by a sequence X, where X is 5′-GCCGUUAGGC-3′ (SEQ ID NO 2236) or any other stem II region known in the art or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers may be present that do not interfere with the function of the nucleic acid molecule.

Sequence X can be a linker of >2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker.

In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide linker” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. The term “non-nucleotide” further refers to any group or compound which can be incorporated into a nucleic acid chain in the place of one or more nucleotide units, including either sugar and/or phosphate substitutions and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties and having enzymatic activity to cleave an RNA or DNA molecule.

In another aspect of the invention, ribozymes or antisense molecules that interact with target RNA molecules and inhibit GRID activity (e.g., inhibit GRID gene) are expressed from transcription units inserted into DNA or RNA vectors. The recombinant vectors are preferably DNA plasmids or viral vectors. Ribozyme or antisense expressing viral vectors can be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors can be used that provide for transient expression of ribozymes or antisense. Such vectors can be repeatedly administered as necessary. Once expressed, the ribozymes or antisense bind to the target RNA and inhibit its function or expression. Delivery of ribozyme or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed endogenously via the use of a single stranded DNA intracellular expression vector.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the individual catalytic activity or stability of the nucleic acid molecule can be decreased (i.e., less than ten-fold), but the overall activity of the nucleic acid molecule is enhanced in vivo.

The nucleic acid molecules of the instant invention, individually, or in combination or in conjunction with other drugs, can be used to treat diseases or conditions discussed above. For example, to treat a disease or condition associated with the levels of GRID, the patient can be treated, or other appropriate cells can be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules can be used in combination with one or more known therapeutic agents to treat tissue/graft rejection, leukemia and/or other disease states or conditions which respond to the modulation of GRID expression.

In another preferred embodiment, the invention features nucleic acid-based inhibitors (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of genes (e.g., GRID) related to the progression and/or maintenance of tissue/graft rejection, leukemia and/or other disease states or conditions which respond to the modulation of GRID expression.

In another aspect, the invention provides mammalian cells containing one or more nucleic acid molecules and/or expression vectors of this invention. The one or more nucleic acid molecules can independently be targeted to the same or different sites.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

### DESCRIPTION OF THE PREFERRED EMBODIMENTS

First the drawings will be described briefly.

Drawings

FIG. 1 shows the secondary structure model for seven different classes of enzymatic nucleic acid molecules. Arrow indicates the site of cleavage . . . indicate the target sequence. Lines interspersed with dots are meant to indicate tertiary interactions.—is meant to indicate base-paired interaction. Group I Intron: P1-P9.0 represent various stem-loop structures (Cech et al., 1994, Nature Struc. Bio., 1, 273). RNase P (M1RNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: : I-IV are meant to indicate four stem-loop structures (Been et al., U.S. Pat. No. 5,625,047). Hammerhead Ribozyme:: I-III are meant to indicate three stem-loop structures; stems I-III can be of any length and can be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 can be covalently linked by one or more bases (i.e., r is >1 base). Helix 1, 4 or 5 can also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides can be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more can be modified chemically (abasic, base, sugar and/or phosphate modifications) or replaced with another base without significant effect. Helix 4 can be formed from two separate molecules, i.e., without a connecting loop. The connecting loop when present can be a ribonucleotide with or without modifications to its base, sugar or phosphate. “q”≧is 2 bases. The connecting loop can also be replaced with a non-nucleotide linker molecule. H refers to bases A, U, or C. Y refers to pyrimidine bases. “______” refers to a covalent bond. (Burke et al, 1996, Nucleic Acids & Mol. Biol., 10, 129; Chowrira et al., U.S. Pat. No. 5,631,359).

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 3 shows an example of the Amberzyme ribozyme motif that is chemically stabilized (see, for example, Beigelman et al., International PCT publication No. WO 99/55857, incorporated by reference herein; also referred to as Class I Motif). The Amberzyme motif is a class of enzymatic nucleic molecules that do not require the presence of a ribonucleotide (2′-OH) group for its activity.

FIG. 4 shows an example of the Zinzyme A ribozyme motif that is chemically stabilized (Beigelman et al., International PCT publication No. WO 99/55857, incorporated by reference herein; also referred to as Class A or Class II Motif). The Zinzyme motif is a class of enzymatic nucleic molecules that do not require the presence of a ribonucleotide (2′-OH) group for its activity.

FIG. 5 shows an example of a DNAzyme motif described by Santoro et al., 1997, PNAS, 94, 4262.

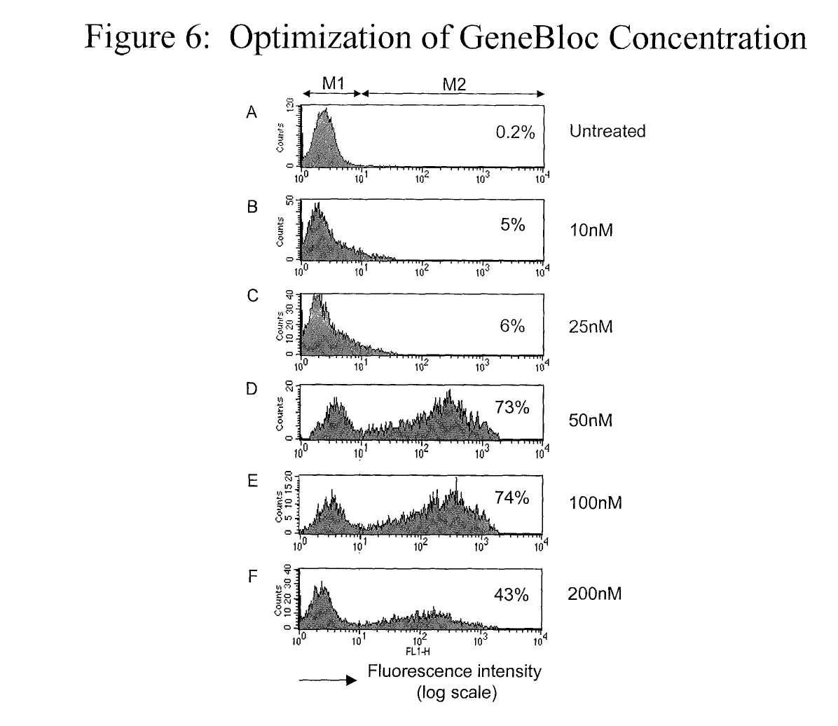

FIG. 6 shows a graph of optimization of GeneBloc concentration. A fluoresceinated randomized antisense GeneBloc (fGB) was used as a marker for uptake using a fixed concentration of lipid. Cells were either untreated (A) or treated continuously for 24hrs with 10-200 nM antisense GeneBloc (B-F). Following treatment, cells were analyzed by flow cytometry. Gate M1 represents either untransfected cells or cells refractory to transfection. Gate M2 represents the transfected cells.

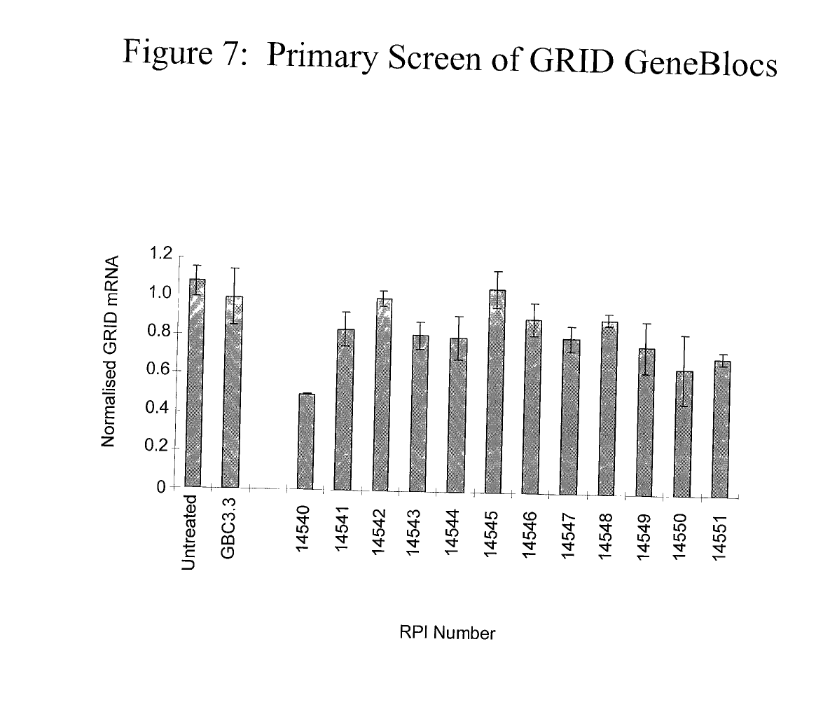

FIG. 7 shows a bar graph of a primary screen of twelve GRID GeneBlocs. Taqman mRNA assay was used to quantify the level of GRID transcript in Jurkat cells treated continuously for 24 hours with 100 nM antisense GeneBloc and 5.0 μgml⁻¹ cationic lipid. For comparison, all data was normalized to the level of β-actin. Error bars represent the standard error of the mean of triplicate points.

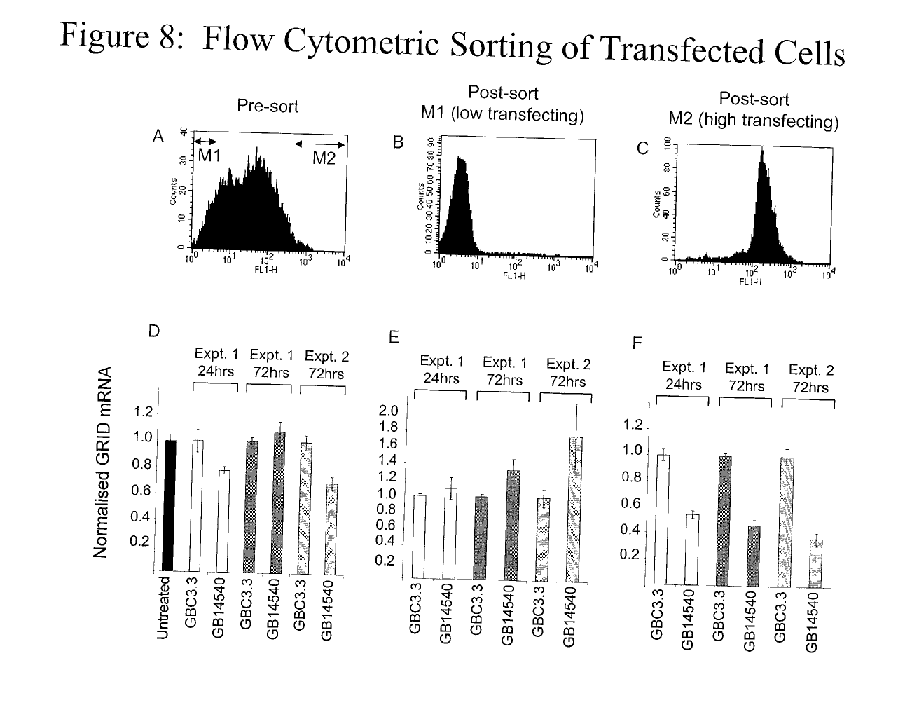

FIG. 8 shows a graph demonstrating that flow cytometric sorting of transfected cells improves antisense GeneBloc mediated inhibition of GRID mRNA expression. Jurkat cells were treated continuously for 24 and 72 hours with GB14540 (75 nM) or control GeneBloc GBC3.3 (75 nM) spiked with 25 nM fluorescent randomized GeneBloc (A) to facilitate the identification of transfected cells. After transfection, the 10% most and least fluorescent cells (gates M2 and M1 respectively) were sorted on a FACStar Plus. Post-sort low transfecting (B) and high transfecting (C) fractions were re-analyzed for purity. Histograms A-D are representative of results obtained in all experiments and were taken from cells treated for 72 hours. The GRID mRNA content of all samples was quantified by Taqman RNA assay and normalized to the β-actin content. For the purposes of inter-experiment comparison, all GB14540 values were also normalized to the appropriate control GBC3.3 value. (D) Normalized GRID mRNA levels in pre-sort samples; (E) Normalized GRID mRNA levels in the post-sort low transfecting fraction; (F) Normalized GRID mRNA levels in the post-sort high transfecting fraction. Error bars represent the range of duplicate points.

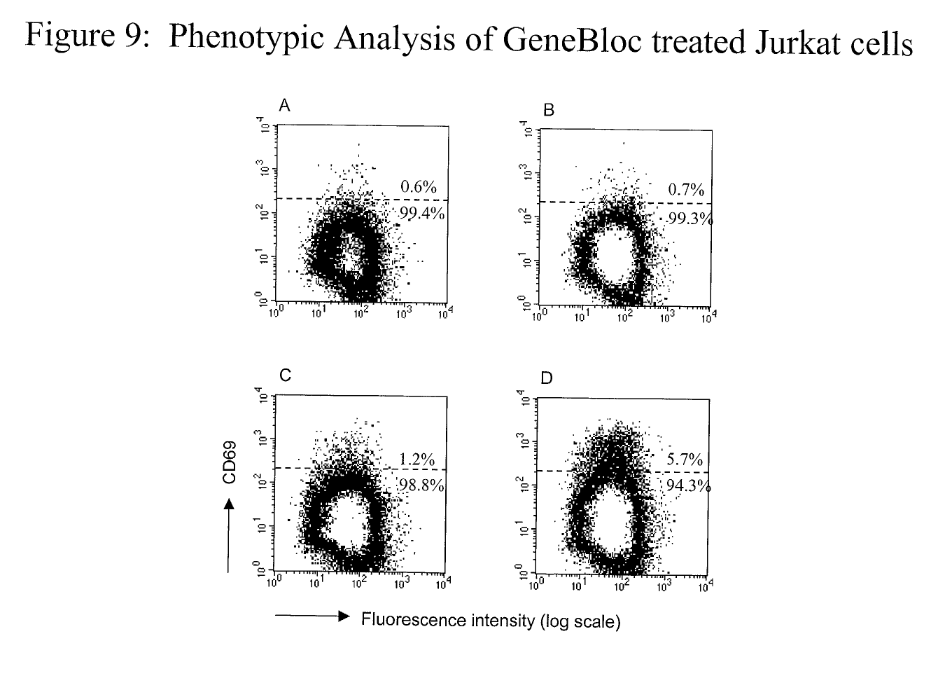

FIG. 9 shows a graph representing the phenotypic analysis of antisense GeneBloc treated Jurkat cells following activation with anti-CD3 and anti-CD28 anti-sera. Jurkat cells were treated continuously for 72 hours with the anti-GRID reagent GB14540 (A, C) and the mismatch control reagent GB17477 (B, D), activated for 22 hours (C, D) and stained for the surface activation marker CD69. Unactivated samples are shown in (A, B).

### MECHANISM OF ACTION OF NUCLEIC ACID MOLECULES OF THE INVENTION

Antisense:

Antisense molecules can be modified or unmodified RNA, DNA, or mixed polymer oligonucleotides which primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oneogenesis 7, 151-190).

In addition, binding of single stranded DNA to RNA can result in nuclease degradation of the heteroduplex (Wu-Pong, supra; Crooke, supra). To date, the only backbone modified DNA chemistry known to act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed endogenously in vivo via the use of a single stranded DNA intracellular expression vector or equivalents and variations thereof.

Triplex Forming Oligonucleotides (TFO):

Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism can result in gene expression or cell death since binding may be irreversible (Mukhopadhyay & Roth, supra).

2-5A Antisense Chimera:

The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid:

Several varieties of naturally occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9,1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention can block to some extent GRID protein expression and can be used to treat disease or diagnose disease associated with levels of GRID.

The enzymatic nature of an enzymatic nucleic acid has significant advantages, such as the concentration of enzymatic nucleic acid necessary to affect a therapeutic treatment is lower. This advantage reflects the ability of the enzymatic nucleic acid to act enzymatically. Thus, a single enzymatic nucleic acid molecule is able to cleave many molecules of target RNA. In addition, the enzymatic nucleic acid is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of an enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript and achieve efficient cleavage in vitro (Zaug et al., 324, Nature 429 1986 ; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

The nucleic acid molecules of the instant invention are also referred to as GeneBloc reagents, which are essentially nucleic acid molecules (e.g., ribozymes, antisense) capable of down-regulating gene expression.

GeneBlocs are modified oligonucleotides, including ribozymes and modified antisense oligonucleotides, that bind to and target specific mRNA molecules. Because GeneBlocs can be designed to target any specific mRNA, their potential applications are quite broad. Traditional antisense approaches have often relied heavily on the use of phosphorothioate modifications to enhance stability in biological samples, leading to a myriad of specificity problems stemming from non-specific protein binding and general cytotoxicity (Stein, 1995, Nature Medicine, 1, 1119). In contrast, GeneBlocs contain a number of modifications that confer nuclease resistance while making minimal use of phosphorothioate linkages, which reduces toxicity, increases binding affinity, and minimizes non-specific effects compared with traditional antisense oligonucleotides. Similar reagents have recently been utilized successfully in various cell culture systems (Vassar, et al., 1999, Science, 286, 735) and in vivo (Jarvis et al., manuscript in preparation). In addition, novel cationic lipids can be utilized to enhance cellular uptake in the presence of serum. Since ribozymes and antisense oligonucleotides regulate gene expression at the RNA level, the ability to maintain a steady-state dose of GeneBloc over several days is important for target protein and phenotypic analysis. The advances in resistance to nuclease degradation and prolonged activity in vitro have supported the use of GeneBlocs in target validation applications.

Target Sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468. All of these publications are hereby incorporated by reference herein in their totality. Other examples include the following PCT applications, which concern inactivation of expression of disease-related genes: WO 95/23225, WO 95/13380, WO 94/02595, all of which are incorporated by reference herein. Rather than repeat the guidance provided in those documents here, specific examples of such methods are provided herein, not limiting to those in the art. Ribozymes and antisense to such targets are designed as described in those applications and synthesized to be tested in vitro and in vivo, as also described. The sequences of human GRID RNAs were screened for optimal enzymatic nucleic acid and antisense target sites using a computer-folding algorithm. Antisense, hammerhead, DNAzyme, NCH, amberzyme, zinzyme, or G-Cleaver ribozyme binding/cleavage sites were identified. These sites are shown in Tables III to VIII and X (all sequences are 5′ to 3′ in the tables; underlined regions can be any sequence or linker X as previously defined herein, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted ribozymes are also useful to test efficacy of action of the enzymatic nucleic acid molecule and/or antisense prior to testing in humans.

Antisense, hammerhead, DNAzyme, NCH, amberzyme, zinzyme or G-Cleaver ribozyme binding/cleavage sites were identified. The nucleic acid molecules were individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions, such as between the binding arms and the catalytic core, were eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; Wincott et al., 1995 Nucleic Acids Res. 23, 2677-2684; and Caruthers et al., 1992, Methods in Enzymology 211,3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can be similarly synthesized.

Oligonucleotides (e.g.; antisense GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684 and Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G₅ and a U for A₁₄ (numbering from Hertel, K. J., et al., 1992, Nucleic Acids Res, 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the examples described above including but not limited to 96-well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., supra, the totality of which is hereby incorporated herein by reference) and are re-suspended in water.

The sequences of the ribozymes and antisense constructs that are chemically synthesized, useful in this study, are shown in Tables III to X. Those in the art will recognize that these sequences are representative only of many more such sequences where the enzymatic portion of the ribozyme (all but the binding arms) is altered to affect activity. The ribozyme and antisense construct sequences listed in Tables III to X can be formed of ribonucleotides or other nucleotides or non-nucleotides. Such ribozymes with enzymatic activity are equivalent to the ribozymes described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein). All these references are incorporated by reference herein. Modifications which enhance their efficacy in cells, and removal of bases from nucleic acid molecules to shorten oligonucleotide synthesis times and reduce chemical requirements are preferably desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modifications of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into ribozymes without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore, when designing nucleic acid molecules the amount of these internucleotide linkages should be minimized. The reduction in the concentration of these linkages should lower toxicity resulting in increased efficacy and higher specificity of these molecules.

Use of the nucleic acid-based molecules of the invention can lead to improved treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

Therapeutic nucleic acid molecules (e.g., enzymatic nucleic acid molecules and antisense nucleic acid molecules) delivered exogenously should preferably be stable within cells until translation of the target RNA has been inhibited long enough to reduce the levels of the undesirable protein. This period of time varies between hours to days depending upon the disease state. The nucleic acid molecules should be resistant to nucleases in order to function as effective intracellular therapeutic agents when delivered exogenously. Improvements in the chemical synthesis of nucleic acid molecules described in the instant invention and in the art (see, e.g., Wincott et al., 1995, Nucleic Acids Res., 23:2677; Carruthers, et al., 1992, Methods in Enzymology, 211:3-19, each incorporated by reference herein) have expanded the ability to modify nucleic acid molecules by introducing nucleotide modifications to enhance their nuclease stability as described above.

In yet another preferred embodiment, nucleic acid catalysts having chemical modifications which maintain or enhance enzymatic activity are provided. Such nucleic acid is also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10 fold (Burgin et al., 1996, Biochemistry, 35, 14090). Such ribozymes herein are said to “maintain” the enzymatic activity of an all RNA ribozyme.

In another aspect the nucleic acid molecules comprise a 5′ and/or a 3′- cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap is selected from the group consisting of inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

Suitable 3′-caps include 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details, see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group which has at least one ring having a conjugated π electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguano sine, N6-methyladeno sine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra).

By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra).

By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In a preferred embodiment, the invention features modified ribozymes with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, (for more details, see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH2 or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference herein in their entireties.

Various modifications to nucleic acid (e.g., antisense and ribozyme) structure can be made to enhance the utility of these molecules. For example, modifications can enhance shelf-life, half-life in vitro, stability, and ease of introduction of such oligonucleotides to the target site, e.g., to enhance penetration of cellular membranes, and confer the ability to recognize and bind to targeted cells.

Use of these molecules can lead to better treatment of the disease progression by affording the possibility of combination therapies (e.g., multiple ribozymes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules can also include combinations of different types of nucleic acid molecules. Therapies can be devised which include a mixture of ribozymes (including different ribozyme motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategiesfor Antisense Oligonucleotide Therapeutics, ed.

Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules can be directly delivered ex vivo to cells or tissues with or without the aforementioned vehicles. Alternatively, the nucleic acid/vehicle combination can be locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (i.e., alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed as described in the art. The compositions of the present invention can also be formulated and used as tablets, capsules or elixirs for oral administration; suppositories for rectal administration; sterile solutions; suspensions for injectable administration; and other compositions known in the art.

The present invention also includes pharmaceutically acceptable formulations of the compounds described. These formulations include salts of the above compounds, e.g., acid addition salts, including salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes that lead to systemic absorption include, without limitations: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). All incorporated by reference herein. Such liposomes have been shown to accumulate selectively in tumors, presumably by extravasation and capture in the neovascularized target tissues (Lasic et al., Science 1995, 267, 1275-1276; Oku et al., 1995, Biochini. Biophys. Acta, 1238, 86-90). All incorporated by reference herein. The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state.

The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention can also be administered to a patient in combination with other therapeutic compounds to increase the overall therapeutic effect. The use of multiple compounds to treat an indication may increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that allow for introduction into the desired target cell (for a review, see Couture et al., 1996, TIG., 12, 510).

In one aspect, the invention features an expression vector comprising a nucleic acid sequence encoding at least one of the nucleic acid molecules disclosed in the instant invention. The nucleic acid sequence encoding the nucleic acid molecule of the instant invention is operable linked in a manner which allows expression of that nucleic acid molecule.

In another aspect, the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given po1 II promoter in a given cell type depends on the nature of the gene regulatory sequences (enhancers, silencers, etc.) present nearby. Prokaryotic RNA polymerase promoters also can be used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein.

Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 45 81-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; and Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; and Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review, see Couture and Stinchcomb, 1996, supra).

In yet another aspect, the invention features an expression vector comprising a nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another preferred embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule.

In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver enzymatic nucleic acid molecules and binding/cleavage sites within GRID RNA.

Nucleic Acid Inhibition of GRID Target RNA

The use of GeneBlocs to modulate the activity of GRID, a putative component of co-stimulatory signaling in T cells, is herein described. An array of GeneBlocs were designed and screened for their ability to reduce GRID mRNA levels whilst leaving transcripts from the closely related genes Grb2 and GRAP unaffected. A series of experiments were conducted to optimize delivery of GeneBlocs to the Jurkat T cell line. Using these conditions, applicant has demonstrated the efficacy of these reagents at both the mRNA and protein level. Anti-CD3/CD28 triggering of Jurkat cells pre-treated with the anti-GRID GeneBloc results in an impairment of CD69 up-regulation consistent with an important role for GRID in transducing the co-stimulatory signal.

### Example 1

### Identification of Potential Target Sites in Human GRID RNA

The sequence of human GRID were screened for accessible sites using a computer-folding algorithm. Regions of the RNA were identified that do not form secondary folding structures. These regions contain potential ribozyme and/or antisense binding/cleavage sites. The sequences of these binding/cleavage sites are shown in Tables III-X.

### Example 2

### Selection of Enzymatic Nucleic Acid Cleavage Sites in Human GRID RNA

Enzymatic nucleic acid target sites are chosen by analyzing sequences of Human GRID (for example, GenBank accession numbers: AJ011736 and Y18051) and prioritizing the sites on the basis of folding. Enzymatic nucleic acids are designed that bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid sequences fold into the appropriate secondary structure. Those enzymatic nucleic acids with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Enzymatic Nucleic Acids and Antisense for Efficient Cleavage and/or Blocking of GRID RNA

Enzymatic nucleic acids and antisense constructs are designed to anneal to various sites in the RNA message. The binding arms of the enzymatic nucleic acids are complementary to the target site sequences described above, while the antisense constructs are fully complimentary to the target site sequences described above. The enzymatic nucleic acids and antisense constructs were chemically synthesized. The method of synthesis used followed the procedure for normal RNA or DNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acids and antisense constructs also can be synthesized from DNA templates using bacteriophage T7 RNA polymerase (Milligan and Uhlenbeck, 1989, Methods Enzymol. 180, 51). Enzymatic nucleic acid and antisense constructs are purified by gel electrophoresis using general methods or are purified by high pressure liquid chromatography (HPLC; see Wincott et al., supra; the totality of which is hereby incorporated herein by reference) and are resuspended in water. The sequences of the chemically synthesized enzymatic nucleic acid and antisense constructs used in this study are shown below in Table III-X.

### Example 4

### Enzymatic Nucleic Acid Cleavage of GRID RNA Target in vitro

Enzymatic nucleic acids targeted to the human GRID RNA are designed and synthesized as described above. These enzymatic nucleic acids can be tested for cleavage activity in vitro, for example, using the following procedure. The target sequences and the nucleotide location within the GRID RNA are given in Tables III-X.

Cleavage Reactions:

Full-length or partially full-length, internally-labeled target RNA for enzymatic nucleic acid cleavage assay is prepared by in vitro transcription in the presence of [a-³²P] CTP, passed over a G 50 Sephadex® column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid in enzymatic nucleic acid cleavage buffer (50 mM Tris-HCl, pH 7.5 at 37° C., 10 mM MgCl₂) and the cleavage reaction was initiated by adding the 2× enzymatic nucleic acid mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., enzymatic nucleic acid excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid cleavage are visualized on an autoradiograph of the gel. The percentage of cleavage is determined by Phosphor Imager® quantitation of bands representing the intact substrate and the cleavage products.

### Example 5

### Nucleic Acid Inhibition of GRID in vivo

Antisense nucleic acid molecules (GeneBlocs) targeted to the human GRID RNA are designed and synthesized as described above. These nucleic acid molecules can be tested for cleavage activity in vivo, for example, using the following procedure. The target sequences and the nucleotide location within the GRID RNA are given in Tables III-X.

GRID shares 60.3% and 57.3% homology at the nucleotide level with the closely related adapter proteins Grb2 and GRAP. In order to discriminate between human GRID and other Grb2 family members, twelve GeneBlocs (see Methods for details) targeting human GRID (GenBank accession number Y18051) were designed, each containing a minimum of six mismatches versus human Grb2 (M96995) and human GRAP (U52518). In order to determine the optimal site for GeneBloc binding and inhibition of the target mRNA, the efficacy of the GeneBlocs was tested on Jurkat cells. A Taqman RNA assay was used to quantify the level of GRID transcript in cells treated continuously for 24hrs. The efficacy of the twelve GeneBlocs, normalized to the levels of a house-keeping gene (β-actin), is shown in FIG. 7. The GeneBloc targeting site 152 (GeneBloc 14540) was the most efficacious, reducing GRID mRNA levels by up to 55% when compared with a randomized control GeneBloc (GBC3.3). To confirm that these effects were target specific, a four base-pair mismatch GeneBloc (GB17477) was synthesized. GRID mRNA expression was unaffected in cells treated with the mismatch control GeneBloc compared to untreated cells.

Efficacy of the Anti-GRID GeneBloc (GB14540) in Jurkat Cells

From the primary screen (FIG. 7), the optimal GeneBloc, GB14540, suppressed GRID mRNA levels by up to 55%. However, this represents the inhibition in a bulk population of cells, some of which are refractory to transfection (see FIG. 6D-F). To investigate the correlation between dose and efficacy, GB14540 was spiked with 25% fGB. Based on mixture experiments with active GeneBlocs in other systems, it was not expected that the presence of the fluorescent GeneBloc would interfere with anti-GRID activity of GB14540. Thus, the most highly fluorescent cells represent the population of cells transfected with the highest concentration of active GeneBloc (‘high transfecting’), whilst the cells that appear to be refractory to transfection should contain a significantly lower concentration active GeneBloc (‘low transfecting’).

Following transfection of a GB14540:fGB mixture, the high transfecting cells (FIG. 8A, Gate M2, the 10% most fluorescent cells) and the low transfecting cells (FIG. 8A, Gate M1, the 10% least fluorescent cells) were purified by FACS sorting. Re-analysis of the sorted cell populations confirmed greater than 95% purity (FIG. 8B-C). Taqman RNA analysis of the treated cells pre- and post-sort (FIG. 8D-F) shows that although GB14540 inhibition of GRID mRNA expression in an unsorted population is variable between experiments (0-30%, FIG. 8D), the level of inhibition is significantly increased to 45-63% in the ‘high transfecting’ fraction (FIG. 8F). In contrast, GRID mRNA levels in the ‘low transfecting’ fraction was similar to that of cells treated with control GBC3.3 (FIG. 8E). These data suggest that the degree of GRID mRNA inhibition is dependent on the dose of GeneBloc delivered to the cells.

To identify the optimal time-point for inhibition of GRID mRNA levels, samples were sorted as described above at 24 and 72 hours following continuous transfection. Analysis of pre- and post-sort samples at these time-points revealed that in pre-sort samples, inhibition of GRID transcript occurred within 24 hours and did not significantly increase throughout the time-course of the experiment (FIG. 8D). In the ‘high transfecting’ fractions, reduction of GRID transcript was ˜45% at 24 hours and increased only fractionally at the 72 hour time-point (50-65%, FIG. 8F). This suggests that GB14540 reduced GRID mRNA levels rapidly following transfection and that inhibition was sustained in the continued presence of GB14540.

Analysis of GRID Protein Levels in GB14540 Treated Cells

To determine whether the reduction in GRID transcript levels was associated with a loss of GRID protein, the level of GRID protein in cells treated continuously with active GeneBloc reagent GB14540 and the mismatch control GB17477 was assessed. When delivered continuously for 72 hours, GB14540 caused a substantial reduction in GRID protein levels as determined by the intensity of the GRID specific band whilst at earlier time-points (24 and 48 hrs) no reduction in protein was observed. Cells treated with the mismatch control GB17477 showed GRID levels comparable to the untreated sample. Cells treated continuously with GB14540 for periods up to 144 hours showed no further reduction in GRID protein levels, suggesting that the effect of the GeneBloc was maximal and sustained from 72 hours onwards. Whilst the effects of the anti-GRID GeneBloc on MRNA levels are seen at 24 hours, the reduction in GRID protein is delayed a further 48 hours indicating that GRID protein may have a relatively long half-life.

The GeneBlocs were designed to target and discriminate GRID from the closely related adapter proteins Grb2 and GRAP. GB14540 contains 6 and 7 mismatches respectively when aligned with the human Grb2 and GRAP sequences. Due to the presence of these mismatches, GB14540 was not expected to inhibit Grb2 mRNA expression. The Western blots used for the GRID assay were stripped and re-probed using an anti-Grb2 antibody. No difference in Grb2 protein levels was observed between the untreated sample and cells treated with either GB14540 or the mismatch control reagent GB17477, confirming that the GB14540 was specific for GRID.

Phenotypic Effects of the Anti-GRID GeneBloc on T Cell Activation

GRID is a novel member of the Grb2 family of adapter proteins. A role for GRID in T cell signaling has been postulated due to its association with known T cell signaling proteins [Law, 1999 #3296][Asada, 1999 #3243][Liu, 1999 #3245] and more recently the T cell co-stimulatory receptor CD28 following activation by cross-linking antibodies (Ellis et al.). To further elucidate the role of GRID in T cell co-stimulatory pathways, applicant studied the expression of early surface activation marker CD69 (Jung et al., 1988, Cellular Immunology, 117, 352, Lanier et al., 1988, J. Exp. Med., 167, 1572) following activation of Jurkat cells treated with GB14540 and GB17477. Jurkat cells were activated by cross-linking anti-CD3 and anti-CD28 monoclonal antibodies using a sub-maximal stimulus to increase the sensitivity of the assay. In cells treated with the mismatch control GeneBloc, GB17477, 5.7% stained CD69 positive following activation compared with 0.7% CD69 positive in unactivated cells (FIG. 9D vs. 9B). In cells treated with the anti-GRID reagent GB14540, there was a marked reduction in the proportion of activated cells, with only 1.3% staining positive for CD69 (FIG. 9C). Expression of CD69 in the unactivated sample remained unaltered at 0.6% (FIG. 9A). As the activation stimulus was increased, the relative difference between the cells treated with GB14540 and GB17477 decreased even though the proportion of cells staining positive for CD69 increased. This can be attributed to the combination of residual GRID protein and supra-maximal activation stimulus. The latter component is particularly relevant to T cell activation since the dependency on co-stimulation is reduced as the strength of the CD3 signal increases (Geppert and Lipsky, 1988, J. Clin. Invest., 81, 1497, Geppert and Lipsky, 1987, Journal of Immunology, 138, 1660).

Taken together, these data suggest that the phenotypic effects described above can be attributed to GRID and not the closely related adapter protein Grb2. The inhibitory effects of GB14540 on CD69 expression support a role for GRID in T cell co-stimulatory signaling.

### Example 6

### Delivery of GeneBloc Reagents to Jurkat Cells

As in many mammalian cell culture systems (Marcusson et al., 1998, Nuc. Acids, Res. 26, 2016), a cationic lipid was found to be necessary to facilitate cellular uptake of oligonucleotide. In preliminary experiments using a fluoresceinated randomized GeneBloc as a marker for uptake, a lipid concentration of 2.5-5.0 μgml⁻¹ was found to be optimal. Although some cells are readily transfected by the GeneBloc, a sub-population of cells remained refractory to transfection (see Gate M2 vs. M1 in FIGS. 6D-6F). In order to minimize the refractory population, the concentration of GeneBloc was varied between 10-200 nM. Transfection frequencies of up to 75% (as determined by fraction of cells in Gate M2) were observed in the 50-100 nM range of GeneBloc concentration. At lower concentrations (10-25nM), the transfection frequency dropped off very steeply whilst at higher concentrations, no further enhancement of transfection was observed. Cationic lipids however are not essential for the use of oligonucleotides in vivo (see McGraw et al., 1997, Anti-Cancer Drug Design, 12, 315-326; Henry et al., 1997, Anti-Cancer Drug Design, 12,409-420).

### Example 7

### Flow Cytometry

Cultures were harvested, washed once and re-suspended in PBS containing 2% FCS. Cells were stained with a human anti-CD69 PE-conjugated antibody (Caltag) using an IgG2a PE-conjugate as an isotype control (Becton Dickinson). Cells were analyzed on a Becton Dickinson FACScan using CellQuest software. Cells were sorted on the basis of fluorescence in the FL1 channel using a Becton Dickinson FACStar Plus. In order to compare the efficiency of GeneBloc uptake using different transfection conditions, a coefficient of transfection was calculated by multiplying the proportion of control GeneBloc (as a fraction of total GeneBloc) and the transfection frequency.

### Example 8

### Protein Studies

Actively growing Jurkat cells (0.1-1.0×10⁶) were harvested, washed once in PBS and re-suspended in 25 μl PBS. Cells were lysed by the addition of an equal volume of ice-cold 2× RIPA buffer (2% NP40, 1.0% sodium deoxycholate, 0.2% SDS in PBS with 2× protease and phosphatase inhibitors). Following a 30 minute incubation on ice, cell debris was removed by centrifugation and the supernatant denatured at 100° C. for 5 minutes following the addition of an equal volume of 2× SDS protein sample buffer. Prior to separation by SDS-PAGE electrophoresis, protein content was normalized using a Coomassie™ Plus-200 protein assay reagent (Pierce). For Western blotting, SDS-PAGE gels were transferred to PVDF membrane (Millipore). Antisera specific for GRID (rabbit polyclonal courtesy of Claire Ashman, GlaxoWellcome), p85 sub-unit of PI-3-kinase (#06-195, Upstate Biotechnology) and Grb2 (sc-255, Santa Cruz) were used as primary antibodies with an anti-rabbit HRP conjugate as the secondary antibody. Bound antibody was visualized using the SuperSignal® West Dura chemiluminescent reagent. For re-probing, chemiluminescent substrate and bound antibody were removed with TBST (TBS +0.5% Tween-20) and ImmunoPure® IgG Elution Buffer (Pierce) respectively.

### Example 9

### Cell Culture

Human Jurkat cell lines E6.1 and J6 were maintained at 37° C. in 5% CO₂ in flasks in RPMI 1641 (+25 mM HEPES) supplemented with 10% fetal calf serum and glutamine. Cells were passaged at a density of 1×10⁶ cells ml⁻¹. GeneBlocs were delivered to the cells using a modified centrifugation-based transfection protocol (Verma et al., 1998, BioTechniques, 25, 46). Cells were grown to a density of 1×10⁶ cells ml⁻¹, harvested by centrifugation and re-suspended in fresh media at 0.75×10⁶ cells ml⁻¹. GeneBloc at 10× final concentration and cationic lipid (25 μgml⁻¹) at 10× final concentration were prepared separately in RPMI media (no FCS or glutamine), mixed 1:1 and incubated at 37° C. for 30 minutes. 1.6 ml aliquots of the cell suspension was dispensed into a 6-well tissue-culture treated plate and 0.4 ml of the GeneBloc:lipid mixture added drop-wise. The GeneBloc:lipid solution was evenly distributed by gentle agitation. Following centrifugation at 1000 rpm for 60 minutes at room temperature, the 6-well plates were incubated for 24-72 hours at 37° C.

### Example 10

### Real-time Quantitative PCR (Taqman)

Human GRID oligonucleotide Taqman probe 6FAM-(5′-ACTCCAGTTTCCCAAATGGTTTCACGAA-3′) (SEQ ID NO 2237)-TAMRA and human actin Taqman probe JOE-(5′-TCGAGCACGGCATCGTCACCAA-3′) (SEQ ID NO 2238)-TAMRA were purchased from PE Applied Biosystems. GRID primers (forward, 5′-AGGATATGTGCCCAAGAATTTCATA-3′) (SEQ ID NO 2239) and reverse, (5′-TGCCTGGTGTCGAGAGAGG-3′) (SEQ ID NO 2240) and actin primers (forward, 5′-GCATGGGTCAGAAGGATTCCTAT-3′) (SEQ ID NO 2241) and reverse, (5′-TGTAGAAGGTGTGGTGCCAGATT-3′) (SEQ ID NO 2242) were purchased from Life Technologies. The Taqman probes were labeled with a reporter dye (FAM or JOE) at the 5′ termini and a quencher dye (TAMRA) at their 3′ termini. A combination RT-PCR and Taqman PCR was performed for each sample in triplicate on an ABI PRISM 7700 Sequence Detection System using the following program: 48° C. for 30 minutes, 95° C. for 10 minutes and then 40 cycles of 95° C. for 15 seconds and 60° C. for 1 minute. The reaction was performed in a total volume of 40 μl with each tube containing 10 U RNase inhibitor (Promega), 1.25 U Amplitaq Gold (PE Biosystems), 100 nM of the GRID and Actin primers, 100 nM GRID FAM Taqman probe, 100 nM Actin JOE Taqman probe and 10 U MuLV reverse transcriptase. PCR Buffer (PE Biosystems #4304441) and dNTPs (PE Biosystems #N808-0261) were added according to the manufacturer's guidelines. A standard curve was generated using serially diluted purified RNA (300, 100, 33 and 11 ng) prepared from untreated Jurkat cells.

### Example 11

### RNA Isolation

Total RNA was isolated from Jurkat J6 or Jurkat E6.1 cells using the 96-well RNeasy kit (Qiagen) and a minor modification of their protocol. 90 μl of RLT buffer was added to each sample, followed by an equal volume of 70% ethanol. Samples were mixed and transferred to a RNeasy-96-plate. A vacuum was applied for 15-60 sec until the wells were dry. 80 μl of 1× DNase solution was added (40 mM Tris-HCl pH 7.5, 10 mM MgCl₂, 10 mM CaCl₂, 10 mM NaCl, 1.2 U/μl RNase-free DNase I). Following incubation at room temperature for 15 minutes, 1 ml of Buffer RW1 was added and incubated for a further 5 minutes. The buffer was removed by applying a vacuum. The wells were washed once in 1 ml of RPE. A second 1 ml aliquot of Buffer RPE was added and the RNeasy-96-plate centrifuged at 6000 rpm for 10 minutes. The RNA was eluted by the addition of 100 ml of RNase-free water. Following incubation at room temperature for 1 minute, the RNA was recovered by centrifugation at 6000 rpm for 4 minutes and stored at −70° C.

Indications

Particular conditions and disease states that can be associated with GRID expression modulation include, but are not limited to. tissue/graft rejection and cancer, such as leukemia.

The present body of knowledge in GRID research indicates the need for methods to assay GRID activity and for compounds that can regulate GRID expression for research, diagnostic, and therapeutic use.

Radiation, chemotherapeutic treatments, and Cyclosporin are non-limiting examples of compounds and/or methods that can be combined with or used in conjunction with the nucleic acid molecules (e.g. ribozymes and antisense molecules) of the instant invention. Those skilled in the art will recognize that other drug compounds and therapies can be similarly be readily combined with the nucleic acid molecules of the instant invention (e.g. ribozymes and antisense molecules) are hence within the scope of the instant invention.

Diagnostic Uses

The nucleic acid molecules of this invention (e.g., ribozymes) can be used as diagnostic tools to examine genetic drift and mutations within diseased cells or to detect the presence of GRID RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one can map nucleotide changes which are important to RNA structure and function in vitro, as well as in cells and tissues. Cleavage of target RNAs with ribozymes can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention include detection of the presence of mRNAs associated with GRID-related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme is used to identify mutant RNA in the sample. As reaction controls, synthetic substrates of both wild-type and mutant RNA are cleaved by both ribozymes to demonstrate the relative ribozyme efficiencies in the reactions and the absence of cleavage of the “non-targeted” RNA species. The cleavage products from the synthetic substrates also serve to generate size markers for the analysis of wild-type and mutant RNAs in the sample population. Thus, each analysis can require two ribozymes, two substrates and one unknown sample, which are combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., GRID) is adequate to establish risk. If probes of comparable specific activity are used for both transcripts, then a qualitative comparison of RNA levels is adequate and will decrease the cost of the initial diagnosis. Higher mutant form to wild-type ratios are correlated with higher risk whether RNA levels are compared qualitatively or quantitatively.

Additional Uses

Potential usefulness of sequence-specific enzymatic nucleic acid molecules of the instant invention have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate
cleavage products with 3′-OH and 5′-guanosine.
Additional protein cofactors required in some cases to help folding and
maintenance of the active structure.
Over 300 known members of this class. Found as an intervening
sequence in Tetrahymena thermophila rRNA, fungal mitochondria,
chloroplasts, phage T4, blue-green algae, and others.
Major structural features largely established through phylogenetic
comparisons, mutagenesis, and biochemical studies [ⁱ,ⁱⁱ].
Complete kinetic framework established for one ribozyme [ⁱⁱⁱ,^{iv},^{v},^{vi}].
Studies of ribozyme folding and substrate docking underway [^{vii},^{viii},^{ix}].
Chemical modification investigation of important residues well
established [^{x},^{xi}].
The small (4-6 nt) binding site may make this ribozyme too non-specific
for targeted RNA cleavage, however, the Tetrahymena group I intron
has been used to repair a “defective” beta-galactosidase message by
the ligation of new beta-galactosidase sequences onto the defective
message[^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺-OH to generate cleavage
products with 3′-OH and 5′-phosphate.
RNAse P is found throughout the prokaryotes and eukaryotes. The
RNA subunit has been sequenced from bacteria, yeast, rodents, and
primates.
Recruitment of endogenous RNAse P for therapeutic applications is
possible through hybridization of an External Guide Sequence (EGS) to
the target RNA [^{xiv},^{xv}]
Important phosphate and 2′ OH contacts recently identified [^{xvi},^{xvii}]
Group II Introns
Size: >1000 nucleotides.
Trans cleavage of target RNAs recently demonstrated [^{xviii},^{xix}]
Sequence requirements not fully determined.
Reaction mechanism: 2′-OH of an internal adenosine generates cleavage
products with 3′-OH and a “lariat” RNA containing a 3′-5′ and a 2′-5′
branch point.
Only natural ribozyme with demonstrated participation in DNA
cleavage [^{xx},^{xxi}] in addition to RNA cleavage and ligation.
Major structural features largely established through phylogenetic
comparisons [^{xxii}].
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate
cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate
cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal
structures [^{xxvi},^{xxvii}]
Minimal ligation activity demonstrated (for engineering through in
vitro selection) [^{xxviii}]
Complete kinetic framework established for two or more ribozymes [^{xxix}].
Chemical modification investigation of important residues well
established [^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable
number to the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined [^{xxxi},^{xxxii},^{xxxiii},^{xxxiv}]
Ligation activity (in addition to cleavage activity) makes ribozyme
amenable to engineering through in vitro selection [^{xxxv}]
Complete kinetic framework established for one ribozyme [^{xxxvi}].
Chemical modification investigation of important residues begun
[^{xxxvii},^{xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined,
although no sequences 5′ of cleavage site are required. Folded
ribozyme contains a pseudoknot structure [^{xl}].
Reaction mechanism: attack by 2′-OH 5′ to the scissile bond to generate
cleavage products with 2′,3′-cyclic phosphate and 5′-OH ends.
Only 2 known members of this class. Found in human HDV.
^{xli}Circular form of HDV is^{xlii} active and shows increased nuclease
stability [^{xliii}]
[ⁱ] Michel, Francois; Westhof, Eric. Slippery substrates. Nat. Struct. Biol. (1994), 1(1), 5-7.
[ⁱⁱ] Lisacek, Frederique; Diaz, Yolande; Michel, Francois. Automatic identification of group I intron cores in genomic DNA sequences. J. Mol. Biol. (1994), 235(4), 1206-17.
[ⁱⁱⁱ] Herschlag, Daniel; Cech, Thomas R. Catalysis of RNA cleavage by the Tetrahymena thermophila ribozyme. 1. Kinetic description of the reaction of an RNA substrate complementary to the active site. Biochemistry (1990), 29(44), 10159-71.
[^{iv}] Herschlag, Daniel; Cech, Thomas R. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
[^{v}] Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
[^{vi}] Bevilacqua, Philip C.; Sugimoto, Naoki; Turner, Douglas H. A mechanistic framework for the second step of splicing catalyzed by the Tetrahymena ribozyme. Biochemistry (1996), 35(2), 648-58.
[^{vii}] Li, Yi; Bevilacqua, Philip C.; Mathews, David; Turner, Douglas H. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
[^{viii}] Banerjee, Aloke Raj; Turner, Douglas H. The time dependence of chemical modification reveals slow steps in the folding of a group I ribozyme. Biochemistry (1995), 34(19), 6504-12.
[^{ix}] Zarrinkar, Patrick P.; Williamson, James R. The P9.1-P9.2 peripheral extension helps guide folding of the Tetrahymena ribozyme. Nucleic Acids Res. (1996), 24(5), 854-8.
[^{x}] Strobel, Scott A.; Cech, Thomas R. Minor groove recognition of the conserved G.cntdot.U pair at the Tetrahymena ribozyme reaction site. Science (Washington, D.C.) (1995), 267(5198), 675-9.
[^{xi}] Strobel, Scott A.; Cech, Thomas R. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
[^{xii}] Sullenger, Bruce A.; Cech, Thomas R. Ribozyme-mediated repair of defective mRNA by targeted trans-splicing. Nature (London) (1994), 371(6498), 619-22.
[^{xiii}] Robertson, H.D.; Altman, S.; Smith, J.D. J. Biol. Chem., 247, 5243-5251 (1972).
[^{xiv}] Forster, Anthony C.; Altman, Sidney. External guide sequences for RNA enzyme. Science (Washington, D.C., 1883-) (1990), 249(4970), 783-6.
[^{xv}] Yuan, Y.; Hwang, E. S.; Altman, S. Targeted cleavage of mRNA by human RNase P. Proc. Natl. Acad. Sci. USA (1992) 89, 8006-10.
[^{xvi}] Harris, Michael E.; Pace, Norman R. Identification of phosphates involved in catalysis by the ribozyme RNase P RNA. RNA (1995), 1(2), 210-18.
[^{xvii}] Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
[^{xviii}] Pyle, Anna Marie; Green, Justin B. Building a Kinetic Framework for Group II Intron Ribozyme Activity: Quantitation of Interdomain Binding and Reaction Rate. Biochemistry (1994), 33(9), 2716-25.
[^{xix}] Michels, William J. Jr.; Pyle, Anna Marie. Conversion of a Group II Intron into a New Multiple-Turnover Ribozyme that Selectively Cleaves Oigonucleotides: Elucidation of Reaction Mechanism and Structure/Function Relationships. Biochemistry (1995), 34(9), 2965-77.
[^{xx}] Zimmerly, Steven; Guo, Huatao; Eskes, Robert; Yang, Jian; Perlman, Philip S.; Lambowitz, Alan M. A group II intron RNA is a catalytic component of a DNA endonuclease involved in intron mobility. Cell (Cambridge, Mass.) (1995), 83(4), 529-38.
[^{xxi}] Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
[^{xxii}] Michel, Francois; Ferat, Jean Luc. Structure and activities of group II introns. Annu. Rev. Biochem. (1995), 64, 435-61.
[^{xxiii}] Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
[^{xxiv}] Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
[^{xxv}] Guo, Hans C. T.; Collins, Richard A. Efficient trans-cleavage of a stem-loop RNA substrate by a ribozyme derived from Neurospora VS RNA. EMBO J. (1995), 14(2), 368-76.
[^{xxvi}] Scoff, W.G., Finch, J.T., Aaron, K. The crystal structure of an all RNA hammerhead ribozyme:A proposed mechanism for RNA catalytic cleavage. Cell, (1995), 81, 991-1002.
[^{xxvii}] McKay, Structure and function of the hammerhead ribozyme: an unfinished story. RNA, (1996), 2, 395-403.
[^{xxviii}] Long, D., Uhlenbeck, O., Hertel, K. Ligation with hammerhead ribozymes. U.S. Pat. No. 5,633,133.
[^{xxix}] Hertel, K.J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
[^{xxx}] Beigelman, L., et al., Chemical modifications of hammerhead ribozymes. J. Biol. Chem., (1995) 270, 25702-25708.
[^{xxxi}] Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.
[^{xxxii}] Chowrira, Bharat M.; Berzal-Herranz, Alfredo; Burke, John M. Novel guanosine requirement for catalysis by the hairpin ribozyme. Nature (London) (1991), 354(6351), 320-2.
[^{xxxiii}] Berzal-Herranz, Alfredo; Joseph, Simpson; Chowrira, Bharat M.; Butcher, Samuel E.; Burke, John M. Essential nucleotide sequences and secondary structure elements of the hairpin ribozyme. EMBO J. (1993), 12(6), 2567-73.
[^{xxxiv}] Joseph, Simpson; Berzal-Herranz, Alfredo; Chowrira, Bharat M.; Butcher, Samuel E. Substrate selection rules for the hairpin ribozyme determined by in vitro selection, mutation, and analysis of mismatched substrates. Genes Dev. (1993), 7(1), 130-8.
[^{xxxv}] Berzal-Herranz, Alfredo; Joseph, Simpson; Burke, John M. In vitro selection of active hairpin ribozymes by sequential RNA-catalyzed cleavage and ligation reactions. Genes Dev. (1992), 6(1), 129-34.
[^{xxxvi}] Hegg, Lisa A.; Fedor, Martha J. Kinetics and Thermodynamics of Intermolecular Catalysis by Hairpin Ribozymes. Biochemistry (1995), 34(48), 15813-28.
[^{xxxvii}] Grasby, Jane A.; Mersmann, Karin; Singh, Mohinder; Gait, Michael J. Purine Functional Groups in Essential Residues of the Hairpin Ribozyme Required for Catalytic Cleavage of RNA. Biochemistry (1995), 34(12), 4068-76.
[^{xxxviii}] Schmidt, Sabine; Beigelman, Leonid; Karpeisky, Alexander; Usman, Nassim; Sorensen, Ulrik S.; Gait, Michael J. Base and sugar requirements for RNA cleavage of essential nucleoside residues in internal loop B of the hairpin ribozyme: implications for secondary structure. Nucleic Acids Res. (1996), 24(4), 573-81.
[^{xxxix}] Perrotta, Anne T.; Been, Michael D. Cleavage of oligoribonucleotides by a ribozyme derived from the hepatitis .delta. virus RNA sequence. Biochemistry (1992), 31(1), 16-21.
[^{xl}] Perrotta, Anne T.; Been, Michael D. A pseudoknot-like structure required for efficient self-cleavage of hepatitis delta virus RNA. Nature (London) (1991), 350(6317), 434-6.
^{xli}
^{xlii}
[^{xliii}] Puttaraju, M.; Perrotta, Anne T.; Been, Michael D. A circular trans-acting hepatitis delta virus ribozyme. Nucleic Acids Res. (1993), 21(18), 4253-8.

TABLE II
A. 2.5 μmol Synthesis Cycle ABI 394 Instrument

| Reagent | Equivalents | Amount | Wait Time* DNA | Wait Time* 2′-O-methyl | Wait Time* RNA |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl Imidazole | 186 | 233 μL | 5 sec | 5 sec | 5 sec |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| Reagent | Equivalents | Amount | Wait Time* DNA | Wait Time* 2′-O-methyl | Wait Time* RNA |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl Imidazole | 1245 | 124 μL | 5 sec | 5 sec | 5 sec |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equivalents: DNA/ | Amount: DNA/ | | Wait Time* | Wait Time* |
|---|---|---|---|---|---|
| Reagent | 2′-O-methyl/Ribo | 2′-O-methyl/Ribo | Wait Time* DNA | 2′-O-methyl | Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl Imidazole | 502/502/502 | 50/50/50 μL | 10 sec | 10 sec | 10 sec |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

*Wait time does not include contact time during delivery.

TABLE III

Human GRID Hammerhead Ribozyme and Substrate Sequence

| | | | | |
|---|---|---|---|---|
| | | Seq | | Seq |
| Pos | Substrate | ID | Ribozyme | ID |

| | | | | |
|---|---|---|---|---|
| 13 | GGCACAGU U AAUGGAUC | 1 | GAUCCAUU CUGAUGAG GCCGUUAGGC CGAA ACUGUGCC | 906 |
| 14 | GCACAGUU A AUGGAUCU | 2 | AGAUCCAU CUGAUGAG GCCGUUAGGC CGAA AACUGUGC | 907 |
| 21 | UAAUGGAU C UGUAAACU | 3 | AGUUUACA CUGAUGAG GCCGUUAGGC CGAA AUCCAUUA | 908 |
| 25 | GGAUCUGU A AACUUGCA | 4 | UGCAAGUU CUGAUGAG GCCGUUAGGC CGAA ACAGAUCC | 909 |
| 30 | UGUAAACU U GCACCCUC | 5 | GAGGGUGC CUGAUGAG GCCGUUAGGC CGAA AGUUUACA | 910 |
| 38 | UGCACCCU C UUUCAGAG | 6 | CUCUGAAA CUGAUGAG GCCGUUAGGC CGAA AGGGUGCA | 911 |
| 40 | CACCCUCU U UCAGAGUG | 7 | CACUCUGA CUGAUGAG GCCGUUAGGC CGAA AGAGGGUG | 912 |
| 41 | ACCCUCUU U CAGAGUGG | 8 | CCACUCUG CUGAUGAG GCCGUUAGGC CGAA AAGAGGGU | 913 |
| 42 | CCCUCUUU C AGAGUGGU | 9 | ACCACUCU CUGAUGAG GCCGUUAGGC CGAA AAAGAGGG | 914 |
| 51 | AGAGUGGU A CAUGGAAG | 10 | CUUCCAUG CUGAUGAG GCCGUUAGGC CGAA ACCACUCU | 915 |
| 76 | AAGUGGAU C CAUACUCU | 11 | AGAGUAUG CUGAUGAG GCCGUUAGGC CGAA AUCCACUU | 916 |
| 80 | GGAUCCAU A CUCUGAAA | 12 | UUUCAGAG CUGAUGAG GCCGUUAGGC CGAA AUGGAUCC | 917 |
| 83 | UCCAUACU C UGAAAUGC | 13 | GCAUUUCA CUGAUGAG GCCGUUAGGC CGAA AGUAUGGA | 918 |
| 95 | AAUGCAGU A ACUCUGAU | 14 | AUCAGAGU CUGAUGAG GCCGUUAGGC CGAA ACUGCAUU | 919 |
| 99 | CAGUAACU C UGAUGCUU | 15 | AAGCAUCA CUGAUGAG GCCGUUAGGC CGAA AGUUACUG | 920 |
| 107 | CUGAUGCU U GAAUUUGU | 16 | ACAAAUUC CUGAUGAG GCCGUUAGGC CGAA AGCAUCAG | 921 |
| 112 | GCUUGAAU U UGUUCUCC | 17 | GGAGAACA CUGAUGAG GCCGUUAGGC CGAA AUUCAAGC | 922 |
| 113 | CUUGAAUU U GUUCUCCC | 18 | GGGAGAAC CUGAUGAG GCCGUUAGGC CGAA AAUUCAAG | 923 |
| 116 | GAAUUUGU U CUCCCUUC | 19 | GAAGGGAG CUGAUGAG GCCGUUAGGC CGAA ACAAAUUC | 924 |
| 117 | AAUUUGUU C UCCCUUCU | 20 | AGAAGGGA CUGAUGAG GCCGUUAGGC CGAA AACAAAUU | 925 |
| 119 | UUUGUUCU C CCUUCUUG | 21 | CAAGAAGG CUGAUGAG GCCGUUAGGC CGAA AGAACAAA | 926 |
| 123 | UUCUCCCU U CUUGCCAG | 22 | CUGGCAAG CUGAUGAG GCCGUUAGGC CGAA AGGGAGAA | 927 |
| 124 | UCUCCCUU C UUGCCAGA | 23 | UCUGGCAA CUGAUGAG GCCGUUAGGC CGAA AAGGGAGA | 928 |
| 126 | UCCCUUCU U GCCAGAAA | 24 | UUUCUGGC CUGAUGAG GCCGUUAGGC CGAA AGAAGGGA | 929 |
| 139 | GAAAGGAU U CUAAUAAC | 25 | GUUAUUAG CUGAUGAG GCCGUUAGGC CGAA AUCCUUUC | 930 |
| 140 | AAAGGAUU C UAAUAACU | 26 | AGUUAUUA CUGAUGAG GCCGUUAGGC CGAA AAUCCUUU | 931 |
| 142 | AGGAUUCU A AUAACUCG | 27 | CGAGUUAU CUGAUGAG GCCGUUAGGC CGAA AGAAUCCU | 932 |
| 145 | AUUCUAAU A ACUCGGUG | 28 | CACCGAGU CUGAUGAG GCCGUUAGGC CGAA AUUAGAAU | 933 |
| 149 | UAAUAACU C GGUGUCAA | 29 | UUGACACC CUGAUGAG GCCGUUAGGC CGAA AGUUAUUA | 934 |
| 155 | CUCGGUGU C AAAGCCAA | 30 | UUGGCUUU CUGAUGAG GCCGUUAGGC CGAA ACACCGAG | 935 |
| 169 | CAAGACAU A AACUCAAU | 31 | AUUGAGUU CUGAUGAG GCCGUUAGGC CGAA AUGUCUUG | 936 |
| 174 | CAUAAACU C AAUCUCUU | 32 | AAGAGAUU CUGAUGAG GCCGUUAGGC CGAA AGUUUAUG | 937 |
| 178 | AACUCAAU C UCUUCUCU | 33 | AGAGAAGA CUGAUGAG GCCGUUAGGC CGAA AUUGAGUU | 938 |
| 180 | CUCAAUCU C UUCUCUUC | 34 | GAAGAGAA CUGAUGAG GCCGUUAGGC CGAA AGAUUGAG | 939 |
| 182 | CAAUCUCU U CUCUUCCA | 35 | UGGAAGAG CUGAUGAG GCCGUUAGGC CGAA AGAGAUUG | 940 |
| 183 | AAUCUCUU C UCUUCCAA | 36 | UUGGAAGA CUGAUGAG GCCGUUAGGC CGAA AAGAGAUU | 941 |
| 185 | UCUCUUCU C UUCCAAAA | 37 | UUUUGGAA CUGAUGAG GCCGUUAGGC CGAA AGAAGAGA | 942 |
| 187 | UCUUCUCU U CCAAAAGC | 38 | GCUUUUGG CUGAUGAG GCCGUUAGGC CGAA AGAGAAGA | 943 |
| 188 | CUUCUCUU C CAAAAGCU | 39 | AGCUUUUG CUGAUGAG GCCGUUAGGC CGAA AAGAGAAG | 944 |
| 197 | CAAAAGCU U CACGUUAC | 40 | GUAACGUG CUGAUGAG GCCGUUAGGC CGAA AGCUUUUG | 945 |
| 198 | AAAAGCUU C ACGUUACA | 41 | UGUAACGU CUGAUGAG GCCGUUAGGC CGAA AAGCUUUU | 946 |
| 203 | CUUCACGU U ACAGCAUG | 42 | CAUGCUGU CUGAUGAG GCCGUUAGGC CGAA ACGUGAAG | 947 |
| 204 | UUCACGUU A CAGCAUGG | 43 | CCAUGCUG CUGAUGAG GCCGUUAGGC CGAA AACGUGAA | 948 |
| 220 | GAAGCUGU U GCCAAGUU | 44 | AACUUGGC CUGAUGAG GCCGUUAGGC CGAA ACAGCUUC | 949 |
| 228 | UGCCAAGU U UGAUUUCA | 45 | UGAAAUCA CUGAUGAG GCCGUUAGGC CGAA ACUUGGCA | 950 |
| 229 | GCCAAGUU U GAUUUCAC | 46 | GUGAAAUC CUGAUGAG GCCGUUAGGC CGAA AACUUGGC | 951 |
| 233 | AGUUUGAU U UCACUGCU | 47 | AGCAGUGA CUGAUGAG GCCGUUAGGC CGAA AUCAAACU | 952 |
| 234 | GUUUGAUU U CACUGCUU | 48 | AAGCAGUG CUGAUGAG GCCGUUAGGC CGAA AAUCAAAC | 953 |
| 235 | UUUGAUUU C ACUGCUUC | 49 | GAAGCAGU CUGAUGAG GCCGUUAGGC CGAA AAAUCAAA | 954 |
| 242 | UCACUGCU U CAGGUGAG | 50 | CUCACCUG CUGAUGAG GCCGUUAGGC CGAA AGCAGUGA | 955 |
| 243 | CACUGCUU C AGGUGAGG | 51 | CCUCACCU CUGAUGAG GCCGUUAGGC CGAA AAGCAGUG | 956 |
| 264 | ACUGAGCU U UCACACUG | 52 | CAGUGUGA CUGAUGAG GCCGUUAGGC CGAA AGCUCAGU | 957 |
| 265 | CUGAGCUU U CACACUGG | 53 | CCAGUGUG CUGAUGAG GCCGUUAGGC CGAA AAGCUCAG | 958 |
| 266 | UGAGCUUU C ACACUGGA | 54 | UCCAGUGU CUGAUGAG GCCGUUAGGC CGAA AAAGCUCA | 959 |
| 280 | GGAGAUGU U UUGAAGAU | 55 | AUCUUCAA CUGAUGAG GCCGUUAGGC CGAA ACAUCUCC | 960 |
| 281 | GAGAUGUU U UGAAGAUU | 56 | AAUCUUCA CUGAUGAG GCCGUUAGGC CGAA AACAUCUC | 961 |
| 282 | AGAUGUUU U GAAGAUUU | 57 | AAAUCUUC CUGAUGAG GCCGUUAGGC CGAA AAACAUCU | 962 |
| 289 | UUGAAGAU U UUAAGUAA | 58 | UUACUUAA CUGAUGAG GCCGUUAGGC CGAA AUCUUCAA | 963 |
| 290 | UGAAGAUU U UAAGUAAC | 59 | GUUACUUA CUGAUGAG GCCGUUAGGC CGAA AAUCUUCA | 964 |
| 291 | GAAGAUUU U AAGUAACC | 60 | GGUUACUU CUGAUGAG GCCGUUAGGC CGAA AAAUCUUC | 965 |
| 292 | AAGAUUUU A AGUAACCA | 61 | UGGUUACU CUGAUGAG GCCGUUAGGC CGAA AAAAUCUU | 966 |
| 296 | UUUUAAGU A ACCAAGAG | 62 | CUCUUGGU CUGAUGAG GCCGUUAGGC CGAA ACUUAAAA | 967 |
| 312 | GGAGUGGU U UAAGGCGG | 63 | CCGCCUUA CUGAUGAG GCCGUUAGGC CGAA ACCACUCC | 968 |
| 313 | GAGUGGUU U AAGGCGGA | 64 | UCCGCCUU CUGAUGAG GCCGUUAGGC CGAA AACCACUC | 969 |
| 314 | AGUGGUUU A AGGCGGAG | 65 | CUCCGCCU CUGAUGAG GCCGUUAGGC CGAA AAACCACU | 970 |
| 325 | GCGGAGCU U GGGAGCCA | 66 | UGGCUCCC CUGAUGAG GCCGUUAGGC CGAA AGCUCCGC | 971 |
| 342 | GGAAGGAU A UGUGCCCA | 67 | UGGGCACA CUGAUGAG GCCGUUAGGC CGAA AUCCUUCC | 972 |
| 356 | CCAAGAAU U UCAUAGAC | 68 | GUCUAUGA CUGAUGAG GCCGUUAGGC CGAA AUUCUUGG | 973 |
| 357 | CAAGAAUU U CAUAGACA | 69 | UGUCUAUG CUGAUGAG GCCGUUAGGC CGAA AAUUCUUG | 974 |
| 358 | AAGAAUUU C AUAGACAU | 70 | AUGUCUAU CUGAUGAG GCCGUUAGGC CGAA AAAUUCUU | 975 |
| 361 | AAUUUCAU A GACAUCCA | 71 | UGGAUGUC CUGAUGAG GCCGUUAGGC CGAA AUGAAAUU | 976 |
| 367 | AUAGACAU C CAGUUUCC | 72 | GGAAACUG CUGAUGAG GCCGUUAGGC CGAA AUGUCUAU | 977 |
| 372 | CAUCCAGU U UCCCAAAU | 73 | AUUUGGGA CUGAUGAG GCCGUUAGGC CGAA ACUGGAUG | 978 |
| 373 | AUCCAGUU U CCCAAAUG | 74 | CAUUUGGG CUGAUGAG GCCGUUAGGC CGAA AACUGGAU | 979 |
| 374 | UCCAGUUU C CCAAAUGG | 75 | CCAUUUGG CUGAUGAG GCCGUUAGGC CGAA AAACUGGA | 980 |
| 384 | CAAAUGGU U UCACGAAG | 76 | CUUCGUGA CUGAUGAG GCCGUUAGGC CGAA ACCAUUUG | 981 |
| 385 | AAAUGGUU U CACGAAGG | 77 | CCUUCGUG CUGAUGAG GCCGUUAGGC CGAA AACCAUUU | 982 |
| 386 | AAUGGUUU C ACGAAGGC | 78 | GCCUUCGU CUGAUGAG GCCGUUAGGC CGAA AAACCAUU | 983 |
| 397 | GAAGGCCU C UCUCGACA | 79 | UGUCGAGA CUGAUGAG GCCGUUAGGC CGAA AGGCCUUC | 984 |
| 399 | AGGCCUCU C UCGACACC | 80 | GGUGUCGA CUGAUGAG GCCGUUAGGC CGAA AGAGGCCU | 985 |
| 401 | GCCUCUCU C GACACCAG | 81 | CUGGUGUC CUGAUGAG GCCGUUAGGC CGAA AGAGAGGC | 986 |
| 420 | AGAGAACU U ACUCAUGG | 82 | CCAUGAGU CUGAUGAG GCCGUUAGGC CGAA AGUUCUCU | 987 |
| 421 | GAGAACUU A CUCAUGGG | 83 | CCCAUGAG CUGAUGAG GCCGUUAGGC CGAA AAGUUCUC | 988 |
| 424 | AACUUACU C AUGGGCAA | 84 | UUGCCCAU CUGAUGAG GCCGUUAGGC CGAA AGUAAGUU | 989 |
| 439 | AAGGAGGU U GGCUUCUU | 85 | AAGAAGCC CUGAUGAG GCCGUUAGGC CGAA ACCUCCUU | 990 |
| 444 | GGUUGGCU U CUUCAUCA | 86 | UGAUGAAG CUGAUGAG GCCGUUAGGC CGAA AGCCAACC | 991 |
| 445 | GUUGGCUU C UUCAUCAU | 87 | AUGAUGAA CUGAUGAG GCCGUUAGGC CGAA AAGCCAAC | 992 |
| 447 | UGGCUUCU U CAUCAUCC | 88 | GGAUGAUG CUGAUGAG GCCGUUAGGC CGAA AGAAGCCA | 993 |
| 448 | GGCUUCUU C AUCAUCCG | 89 | CGGAUGAU CUGAUGAG GCCGUUAGGC CGAA AAGAAGCC | 994 |
| 451 | UUCUUCAU C AUCCGGGC | 90 | GCCCGGAU CUGAUGAG GCCGUUAGGC CGAA AUGAAGAA | 995 |
| 454 | UUCAUCAU C CGGGCCAG | 91 | CUGGCCCG CUGAUGAG GCCGUUAGGC CGAA AUGAUGAA | 996 |
| 471 | CCAGAGCU C CCCAGGGG | 92 | CCCCUGGG CUGAUGAG GCCGUUAGGC CGAA AGCUCUGG | 997 |
| 483 | AGGGGACU U CUCCAUCU | 93 | AGAUGGAG CUGAUGAG GCCGUUAGGC CGAA AGUCCCCU | 998 |
| 484 | GGGGACUU C UCCAUCUC | 94 | GAGAUGGA CUGAUGAG GCCGUUAGGC CGAA AAGUCCCC | 999 |
| 486 | GGACUUCU C CAUCUCUG | 95 | CAGAGAUG CUGAUGAG GCCGUUAGGC CGAA AGAAGUCC | 1000 |
| 490 | UUCUCCAU C UCUGUCAG | 96 | CUGACAGA CUGAUGAG GCCGUUAGGC CGAA AUGGAGAA | 1001 |
| 492 | CUCCAUCU C UGUCAGGC | 97 | GCCUGACA CUGAUGAG GCCGUUAGGC CGAA AGAUGGAG | 1002 |
| 496 | AUCUCUGU C AGGCAUGA | 98 | UCAUGCCU CUGAUGAG GCCGUUAGGC CGAA ACAGAGAU | 1003 |
| 514 | GAUGACGU U CAACACUU | 99 | AAGUGUUG CUGAUGAG GCCGUUAGGC CGAA ACGUCAUC | 1004 |
| 515 | AUGACGUU C AACACUUC | 100 | GAAGUGUU CUGAUGAG GCCGUUAGGC CGAA AACGUCAU | 1005 |
| 522 | UCAACACU U CAAGGUCA | 101 | UGACCUUG CUGAUGAG GCCGUUAGGC CGAA AGUGUUGA | 1006 |
| 523 | CAACACUU C AAGGUCAU | 102 | AUGACCUU CUGAUGAG GCCGUUAGGC CGAA AAGUGUUG | 1007 |
| 529 | UUCAAGGU C AUGCGAGA | 103 | UCUCGCAU CUGAUGAG GCCGUUAGGC CGAA ACCUUGAA | 1008 |
| 548 | ACAAGGGU A AUUACUUU | 104 | AAAGUAAU CUGAUGAG GCCGUUAGGC CGAA ACCCUUGU | 1009 |
| 551 | AGGGUAAU U ACUUUCUG | 105 | CAGAAAGU CUGAUGAG GCCGUUAGGC CGAA AUUACCCU | 1010 |
| 552 | GGGUAAUU A CUUUCUGU | 106 | ACAGAAAG CUGAUGAG GCCGUUAGGC CGAA AAUUACCC | 1011 |
| 555 | UAAUUACU U UCUGUGGA | 107 | UCCACAGA CUGAUGAG GCCGUUAGGC CGAA AGUAAUUA | 1012 |
| 556 | AAUUACUU U CUGUGGAC | 108 | GUCCACAG CUGAUGAG GCCGUUAGGC CGAA AAGUAAUU | 1013 |
| 557 | AUUACUUU C UGUGGACU | 109 | AGUCCACA CUGAUGAG GCCGUUAGGC CGAA AAAGUAAU | 1014 |
| 573 | UGAGAAGU U UCCAUCCC | 110 | GGGAUGGA CUGAUGAG GCCGUUAGGC CGAA ACUUCUCA | 1015 |
| 574 | GAGAAGUU U CCAUCCCU | 111 | AGGGAUGG CUGAUGAG GCCGUUAGGC CGAA AACUUCUC | 1016 |
| 575 | AGAAGUUU C CAUCCCUA | 112 | UAGGGAUG CUGAUGAG GCCGUUAGGC CGAA AAACUUCU | 1017 |
| 579 | GUUUCCAU C CCUAAAUA | 113 | UAUUUAGG CUGAUGAG GCCGUUAGGC CGAA AUGGAAAC | 1018 |
| 583 | CCAUCCCU A AAUAAGCU | 114 | AGCUUAUU CUGAUGAG GCCGUUAGGC CGAA AGGGAUGG | 1019 |
| 587 | CCCUAAAU A AGCUGGUA | 115 | UACCAGCU CUGAUGAG GCCGUUAGGC CGAA AUUUAGGG | 1020 |
| 595 | AAGCUGGU A GACUACUA | 116 | UAGUAGUC CUGAUGAG GCCGUUAGGC CGAA ACCAGCUU | 1021 |
| 600 | GGUAGACU A CUACAGGA | 117 | UCCUGUAG CUGAUGAG GCCGUUAGGC CGAA AGUCUACC | 1022 |
| 603 | AGACUACU A CAGGACAA | 118 | UUGUCCUG CUGAUGAG GCCGUUAGGC CGAA AGUAGUCU | 1023 |
| 614 | GGACAAAU U CCAUCUCC | 119 | GGAGAUGG CUGAUGAG GCCGUUAGGC CGAA AUUUGUCC | 1024 |
| 615 | GACAAAUU C CAUCUCCA | 120 | UGGAGAUG CUGAUGAG GCCGUUAGGC CGAA AAUUUGUC | 1025 |
| 619 | AAUUCCAU C UCCAGACA | 121 | UGUCUGGA CUGAUGAG GCCGUUAGGC CGAA AUGGAAUU | 1026 |
| 621 | UUCCAUCU C CAGACAGA | 122 | UCUGUCUG CUGAUGAG GCCGUUAGGC CGAA AGAUGGAA | 1027 |
| 637 | AAGCAGAU C UUCCUUAG | 123 | CUAAGGAA CUGAUGAG GCCGUUAGGC CGAA AUCUGCUU | 1028 |
| 639 | GCAGAUCU U CCUUAGAG | 124 | CUCUAAGG CUGAUGAG GCCGUUAGGC CGAA AGAUCUGC | 1029 |
| 640 | CAGAUCUU C CUUAGAGA | 125 | UCUCUAAG CUGAUGAG GCCGUUAGGC CGAA AAGAUCUG | 1030 |
| 643 | AUCUUCCU U AGAGACAG | 126 | CUGUCUCU CUGAUGAG GCCGUUAGGC CGAA AGGAAGAU | 1031 |
| 644 | UCUUCCUU A GAGACAGA | 127 | UCUGUCUC CUGAUGAG GCCGUUAGGC CGAA AAGGAAGA | 1032 |
| 671 | ACCAGGGU C ACCGGGGC | 128 | GCCCCGGU CUGAUGAG GCCGUUAGGC CGAA ACCCUGGU | 1033 |
| 699 | CCGGAGGU C CCAGGGAG | 129 | CUCCCUGG CUGAUGAG GCCGUUAGGC CGAA ACCUCCGG | 1034 |
| 718 | CCACACCU C AGUGGGGC | 130 | GCCCCACU CUGAUGAG GCCGUUAGGC CGAA AGGUGUGG | 1035 |
| 742 | GAAGAAAU C CGACCUUC | 131 | GAAGGUCG CUGAUGAG GCCGUUAGGC CGAA AUUUCUUC | 1036 |
| 749 | UCCGACCU U CGAUGAAC | 132 | GUUCAUCG CUGAUGAG GCCGUUAGGC CGAA AGGUCGGA | 1037 |
| 750 | CCGACCUU C GAUGAACC | 133 | GGUUCAUC CUGAUGAG GCCGUUAGGC CGAA AAGGUCGG | 1038 |
| 768 | GAAGCUGU C GGAUCACC | 134 | GGUGAUCC CUGAUGAG GCCGUUAGGC CGAA ACAGCUUC | 1039 |
| 773 | UGUCGGAU C ACCCCCCG | 135 | CGGGGGGU CUGAUGAG GCCGUUAGGC CGAA AUCCGACA | 1040 |
| 787 | CCGACCCU U CCCCUGCA | 136 | UGCAGGGG CUGAUGAG GCCGUUAGGC CGAA AGGGUCGG | 1041 |
| 788 | CGACCCUU C CCCUGCAG | 137 | CUGCAGGG CUGAUGAG GCCGUUAGGC CGAA AAGGGUCG | 1042 |
| 821 | CACAGCCU C CGCAAUAU | 138 | AUAUUGCG CUGAUGAG GCCGUUAGGC CGAA AGGCUGUG | 1043 |
| 828 | UCCGCAAU A UGCCCCAG | 139 | CUGGGGCA CUGAUGAG GCCGUUAGGC CGAA AUUGCGGA | 1044 |
| 873 | GCAGCGAU A UCUGCAGC | 140 | GCUGCAGA CUGAUGAG GCCGUUAGGC CGAA AUCGCUGC | 1045 |
| 875 | AGCGAUAU C UGCAGCAC | 141 | GUGCUGCA CUGAUGAG GCCGUUAGGC CGAA AUAUCGCU | 1046 |
| 890 | ACCACCAU U UCCACCAG | 142 | CUGGUGGA CUGAUGAG GCCGUUAGGC CGAA AUGGUGGU | 1047 |
| 891 | CCACCAUU U CCACCAGG | 143 | CCUGGUGG CUGAUGAG GCCGUUAGGC CGAA AAUGGUGG | 1048 |
| 892 | CACCAUUU C CACCAGGA | 144 | UCCUGGUG CUGAUGAG GCCGUUAGGC CGAA AAAUGGUG | 1049 |
| 919 | GGCAGCCU U GACAUAAA | 145 | UUUAUGUC CUGAUGAG GCCGUUAGGC CGAA AGGCUGCC | 1050 |
| 925 | CUUGACAU A AAUGAUGG | 146 | CCAUCAUU CUGAUGAG GCCGUUAGGC CGAA AUGUCAAG | 1051 |
| 938 | AUGGGCAU U GUGGCACC | 147 | GGUGCCAC CUGAUGAG GCCGUUAGGC CGAA AUGCCCAU | 1052 |
| 951 | CACCGGCU U GGGCAGUG | 148 | CACUGCCC CUGAUGAG GCCGUUAGGC CGAA AGCCGGUG | 1053 |
| 976 | GCGGCCCU C AUGCAUCG | 149 | CGAUGCAU CUGAUGAG GCCGUUAGGC CGAA AGGGCCGC | 1054 |
| 983 | UCAUGCAU C GGAGACAC | 150 | GUGUCUCC CUGAUGAG GCCGUUAGGC CGAA AUGCAUGA | 1055 |
| 1009 | GUGCAGCU C CAGGCGGC | 151 | GCCGCCUG CUGAUGAG GCCGUUAGGC CGAA AGCUGCAC | 1056 |
| 1047 | GGCGCUGU A UGACUUUG | 152 | CAAAGUCA CUGAUGAG GCCGUUAGGC CGAA ACAGCGCC | 1057 |
| 1053 | GUAUGACU U UGAGGCCC | 153 | GGGCCUCA CUGAUGAG GCCGUUAGGC CGAA AGUCAUAC | 1058 |
| 1054 | UAUGACUU U GAGGCCCU | 154 | AGGGCCUC CUGAUGAG GCCGUUAGGC CGAA AAGUCAUA | 1059 |
| 1083 | GCUGGGGU U CCACAGCG | 155 | CGCUGUGG CUGAUGAG GCCGUUAGGC CGAA ACCCCAGC | 1060 |
| 1084 | CUGGGGUU C CACAGCGG | 156 | CCGCUGUG CUGAUGAG GCCGUUAGGC CGAA AACCCCAG | 1061 |
| 1108 | GUGGAGGU C CUGGAUAG | 157 | CUAUCCAG CUGAUGAG GCCGUUAGGC CGAA ACCUCCAC | 1062 |
| 1115 | UCCUGGAU A GCUCCAAC | 158 | GUUGGAGC CUGAUGAG GCCGUUAGGC CGAA AUCCAGGA | 1063 |
| 1119 | GGAUAGCU C CAACCCAU | 159 | AUGGGUUG CUGAUGAG GCCGUUAGGC CGAA AGCUAUCC | 1064 |
| 1128 | CAACCCAU C CUGGUGGA | 160 | UCCACCAG CUGAUGAG GCCGUUAGGC CGAA AUGGGUUG | 1065 |
| 1165 | CUGGGCCU C UUCCCUGC | 161 | GCAGGGAA CUGAUGAG GCCGUUAGGC CGAA AGGCCCAG | 1066 |
| 1167 | GGGCCUCU U CCCUGCCA | 162 | UGGCAGGG CUGAUGAG GCCGUUAGGC CGAA AGAGGCCC | 1067 |
| 1168 | GGCCUCUU C CCUGCCAA | 163 | UUGGCAGG CUGAUGAG GCCGUUAGGC CGAA AAGAGGCC | 1068 |
| 1179 | UGCCAACU A CGUGGCAC | 164 | GUGCCACG CUGAUGAG GCCGUUAGGC CGAA AGUUGGCA | 1069 |
| 1200 | GACCCGAU A AACUCUUC | 165 | GAAGAGUU CUGAUGAG GCCGUUAGGC CGAA AUCGGGUC | 1070 |
| 1205 | GAUAAACU C UUCAGGGG | 166 | CCCCUGAA CUGAUGAG GCCGUUAGGC CGAA AGUUUAUC | 1071 |
| 1207 | UAAACUCU U CAGGGGAC | 167 | GUCCCCUG CUGAUGAG GCCGUUAGGC CGAA AGAGUUUA | 1072 |
| 1208 | AAACUCUU C AGGGGACA | 168 | UGUCCCCU CUGAUGAG GCCGUUAGGC CGAA AAGAGUUU | 1073 |
| 1223 | CAGAAGCU U UUUGUCUG | 169 | CAGACAAA CUGAUGAG GCCGUUAGGC CGAA AGCUUCUG | 1074 |
| 1224 | AGAAGCUU U UUGUCUGG | 170 | CCAGACAA CUGAUGAG GCCGUUAGGC CGAA AAGCUUCU | 1075 |
| 1225 | GAAGCUUU U UGUCUGGA | 171 | UCCAGACA CUGAUGAG GCCGUUAGGC CGAA AAAGCUUC | 1076 |
| 1226 | AAGCUUUU U GUCUGGAG | 172 | CUCCAGAC CUGAUGAG GCCGUUAGGC CGAA AAAAGCUU | 1077 |
| 1229 | CUUUUUGU C UGGAGCUG | 173 | CAGCUCCA CUGAUGAG GCCGUUAGGC CGAA ACAAAAAG | 1078 |
| 1274 | GCUGGACU C CAUGACUA | 174 | UAGUCAUG CUGAUGAG GCCGUUAGGC CGAA AGUCCAGC | 1079 |
| 1282 | CCAUGACU A UAUAUACA | 175 | UGUAUAUA CUGAUGAG GCCGUUAGGC CGAA AGUCAUGG | 1080 |
| 1284 | AUGACUAU A UAUACAUA | 176 | UAUGUAUA CUGAUGAG GCCGUUAGGC CGAA AUAGUCAU | 1081 |
| 1286 | GACUAUAU A UACAUACA | 177 | UGUAUGUA CUGAUGAG GCCGUUAGGC CGAA AUAUAGUC | 1082 |
| 1288 | CUAUAUAU A CAUACAUC | 178 | GAUGUAUG CUGAUGAG GCCGUUAGGC CGAA AUAUAUAG | 1083 |
| 1292 | AUAUACAU A CAUCUAUC | 179 | GAUAGAUG CUGAUGAG GCCGUUAGGC CGAA AUGUAUAU | 1084 |

Input Sequence = HSA011736.
Cut Site = UH/.
Stem Length = 8.
Core Sequence = CUGAUGAG GCCGUUAGGC CGAA
HSA011736 (Homo sapiens mRNA for growth factor receptor binding protein (GRBLG); 1303 bp)

Underlined region can be any X sequence or linker as defined herein.

TABLE IV

Humam GRID NCH Ribozyme and Substrate Sequence

| | | | | |
|---|---|---|---|---|
| | | Seq | | Seq |
| Pos | Substrate | ID | Ribozyme | ID |

| | | | | |
|---|---|---|---|---|
| 10 | GGAGGCAC A GUUAAUGG | 180 | CCAUUAAC CUGAUGAG GCCGUUAGGC CGAA IUGCCUCC | 1085 |
| 22 | AAUGGAUC U GUAAACUU | 181 | AAGUUUAC CUGAUGAG GCCGUUAGGC CGAA IAUCCAUU | 1086 |
| 29 | CUGUAAAC U UGCACCCU | 182 | AGGGUGCA CUGAUGAG GCCGUUAGGC CGAA IUUUACAG | 1087 |
| 33 | AAACUUGC A CCCUCUUU | 183 | AAAGAGGG CUGAUGAG GCCGUUAGGC CGAA ICAAGUUU | 1088 |
| 35 | ACUUGCAC C CUCUUUCA | 184 | UGAAAGAG CUGAUGAG GCCGUUAGGC CGAA IUGCAAGU | 1089 |
| 36 | CUUGCACC C UCUUUCAG | 185 | CUGAAAGA CUGAUGAG GCCGUUAGGC CGAA IGUGCAAG | 1090 |
| 37 | UUGCACCC U CUUUCAGA | 186 | UCUGAAAG CUGAUGAG GCCGUUAGGC CGAA IGGUGCAA | 1091 |
| 39 | GCACCCUC U UUCAGAGU | 187 | ACUCUGAA CUGAUGAG GCCGUUAGGC CGAA IAGGGUGC | 1092 |
| 43 | CCUCUCUC A GAGUGGUA | 188 | UACCACUC CUGAUGAG GCCGUUAGGC CGAA IAAAGAGG | 1093 |
| 53 | AGUGGUAC A UGGAAGAC | 189 | GUCUUCCA CUGAUGAG GCCGUUAGGC CGAA IUACCACU | 1094 |
| 62 | UGGAAGAC A GCACAAAG | 190 | CUUUGUGC CUGAUGAG GCCGUUAGGC CGAA IUCUUCCA | 1095 |
| 65 | AAGACAGC A CAAAGUGG | 191 | CCACUUUG CUGAUGAG GCCGUUAGGC CGAA ICUGUCUU | 1096 |
| 67 | GACAGCAC A AAGUGGAU | 192 | AUCCACUU CUGAUGAG GCCGUUAGGC CGAA IUGCUGUC | 1097 |
| 77 | AGUGGAUC C AUACUCUG | 193 | CAGAGUAU CUGAUGAG GCCGUUAGGC CGAA IAUCCACU | 1098 |
| 78 | GUGGAUCC A UACUCUGA | 194 | UCAGAGUA CUGAUGAG GCCGUUAGGC CGAA IGAUCCAC | 1099 |
| 82 | AUCCAUAC U CUGAAAUG | 195 | CAUUUCAG CUGAUGAG GCCGUUAGGC CGAA IUAUGGAU | 1100 |
| 84 | CCAUACUC U GAAAUGCA | 196 | UGCAUUUC CUGAUGAG GCCGUUAGGC CGAA IAGUAUGG | 1101 |
| 92 | UGAAAUGC A GUAACUCU | 197 | AGAGUUAC CUGAUGAG GCCGUUAGGC CGAA ICAUUUCA | 1102 |
| 98 | GCAGUAAC U CUGAUGCU | 198 | AGCAUCAG CUGAUGAG GCCGUUAGGC CGAA IUUACUGC | 1103 |
| 100 | AGUAACUC U GAUGCUUG | 199 | CAAGCAUC CUGAUGAG GCCGUUAGGC CGAA IAGUUACU | 1104 |
| 106 | UCUGAUGC U UGAAUUUG | 200 | CAAAUUCA CUGAUGAG GCCGUUAGGC CGAA ICAUCAGA | 1105 |
| 118 | AUUUGUUC U CCCUUCUU | 201 | AAGAAGGG CUGAUGAG GCCGUUAGGC CGAA IAACAAAU | 1106 |
| 120 | UUGUUCUC C CUUCUUGC | 202 | GCAAGAAG CUGAUGAG GCCGUUAGGC CGAA IAGAACAA | 1107 |
| 121 | UGUUCUCC C UUCUUGCC | 203 | GGCAAGAA CUGAUGAG GCCGUUAGGC CGAA IGAGAACA | 1108 |
| 122 | GUUCUCCC U UCUUGCCA | 204 | UGGCAAGA CUGAUGAG GCCGUUAGGC CGAA IGGAGAAC | 1109 |
| 125 | CUCCCUUC U UGCCAGAA | 205 | UUCUGGCA CUGAUGAG GCCGUUAGGC CGAA IAAGGGAG | 1110 |
| 129 | CUUCUUGC C AGAAAGGA | 206 | UCCUUUCU CUGAUGAG GCCGUUAGGC CGAA ICAAGAAG | 1111 |
| 130 | UUCUUGCC A GAAAGGAU | 207 | AUCCUUUC CUGAUGAG GCCGUUAGGC CGAA IGCAAGAA | 1112 |
| 141 | AAGGAUUC U AAUAACUC | 208 | GAGUUAUU CUGAUGAG GCCGUUAGGC CGAA IAAUCCUU | 1113 |
| 148 | CUAAUAAC U CGGUGUCA | 209 | UGACACCG CUGAUGAG GCCGUUAGGC CGAA IUUAUUAG | 1114 |
| 156 | UCGGUGUC A AAGCCAAG | 210 | CUUGGCUU CUGAUGAG GCCGUUAGGC CGAA IACACCGA | 1115 |
| 161 | GUCAAAGC C AAGACAUA | 211 | UAUGUCUU CUGAUGAG GCCGUUAGGC CGAA ICUUUGAC | 1116 |
| 162 | UCAAAGCC A AGACAUAA | 212 | UUAUGUCU CUGAUGAG GCCGUUAGGC CGAA IGCUUUGA | 1117 |
| 167 | GCCAAGAC A UAAACUCA | 213 | UGAGUUUA CUGAUGAG GCCGUUAGGC CGAA IUCUUGGC | 1118 |
| 173 | ACAUAAAC U CAAUCUCU | 214 | AGAGAUUG CUGAUGAG GCCGUUAGGC CGAA IUUUAUGU | 1119 |
| 175 | AUAAACUC A AUCUCUUC | 215 | GAAGAGAU CUGAUGAG GCCGUUAGGC CGAA IAGUUUAU | 1120 |
| 179 | ACUCAAUC U CUUCUCUU | 216 | AAGAGAAG CUGAUGAG GCCGUUAGGC CGAA IAUUGAGU | 1121 |
| 181 | UCAAUCUC U UCUCUUCC | 217 | GGAAGAGA CUGAUGAG GCCGUUAGGC CGAA IAGAUUGA | 1122 |
| 184 | AUCUCUUC U CUUCCAAA | 218 | UUUGGAAG CUGAUGAG GCCGUUAGGC CGAA IAAGAGAU | 1123 |
| 186 | CUCUUCUC U UCCAAAAG | 219 | CUUUUGGA CUGAUGAG GCCGUUAGGC CGAA IAGAAGAG | 1124 |
| 189 | UUCUCUUC C AAAAGCUU | 220 | AAGCUUUU CUGAUGAG GCCGUUAGGC CGAA IAAGAGAA | 1125 |
| 190 | UCUCUUCC A AAAGCUUC | 221 | GAAGCUUU CUGAUGAG GCCGUUAGGC CGAA IGAAGAGA | 1126 |
| 196 | CCAAAAGC U UCACGUUA | 222 | UAACGUGA CUGAUGAG GCCGUUAGGC CGAA ICUUUUGG | 1127 |
| 199 | AAAGCUUC A CGUUACAG | 223 | CUGUAACG CUGAUGAG GCCGUUAGGC CGAA IAAGCUUU | 1128 |
| 206 | CACGUUAC A GCAUGGAA | 224 | UUCCAUGC CUGAUGAG GCCGUUAGGC CGAA IUAACGUG | 1129 |
| 209 | GUUACAGC A UGGAAGCU | 225 | AGCUUCCA CUGAUGAG GCCGUUAGGC CGAA ICUGUAAC | 1130 |
| 217 | AUGGAAGC U GUUGCCAA | 226 | UUGGCAAC CUGAUGAG GCCGUUAGGC CGAA ICUUCCAU | 1131 |
| 223 | GCUGUUGC C AAGUUUCA | 227 | UCAAACUU CUGAUGAG GCCGUUAGGC CGAA ICAACAGC | 1132 |
| 224 | CUGUUGCC A AGUUUGAU | 228 | AUCAAACU CUGAUGAG GCCGUUAGGC CGAA IGCAACAG | 1133 |
| 236 | UUGAUUUC A CUCCUUCA | 229 | UGAAGCAG CUGAUGAG GCCGUUAGGC CGAA IAAAUCAA | 1134 |
| 238 | GAUUUCAC U GCUUCAGG | 230 | CCUGAAGC CUGAUGAG GCCGUUAGGC CGAA IUGAAAUC | 1135 |
| 241 | UUCACUGC U UCAGGUGA | 231 | UCACCUGA CUGAUGAG GCCGUUAGGC CGAA ICAGUGAA | 1136 |
| 244 | ACUGCUCC A GGUGAGGA | 232 | UCCUCACC CUGAUGAG GCCGUUAGGC CGAA IAAGCAGU | 1137 |
| 258 | GGAUGAAC U GAGCUUUC | 233 | GAAAGCUC CUGAUGAG GCCGUUAGGC CGAA IUUCAUCC | 1138 |
| 263 | AACUGAGC U UUCACACU | 234 | AGUGUGAA CUGAUGAG GCCGUUAGGC CGAA ICUCAGUU | 1139 |
| 267 | GAGCUUUC A CACUGGAG | 235 | CUCCAGUG CUGAUGAG GCCGUUAGGC CGAA IAAAGCUC | 1140 |
| 269 | GCUUUCAC A CUGGAGAU | 236 | AUCUCCAG CUGAUGAG GCCGUUAGGC CGAA IUGAAAGC | 1141 |
| 271 | UUUCACAC U GGAGAUGU | 237 | ACAUCUCC CUGAUGAG GCCGUUAGGC CGAA IUGUGAAA | 1142 |
| 299 | UAAGUAAC C AAGAGGAG | 238 | CUCCUCUU CUGAUGAG GCCGUUAGGC CGAA IUUACUUA | 1143 |
| 300 | AAGUAACC A AGAGGAGU | 239 | ACUCCUCU CUGAUGAG GCCGUUAGGC CGAA IGUUACUU | 1144 |
| 324 | GGCGGAGC U UGGGAGCC | 240 | GGCUCCCA CUGAUGAG GCCGUUAGGC CGAA ICUCCGCC | 1145 |
| 332 | UUGGGAGC C AGGAAGGA | 241 | UCCUUCCU CUGAUGAG GCCGUUAGGC CGAA ICUCCCAA | 1146 |
| 333 | UGGGAGCC A GGAAGGAU | 242 | AUCCUUCC CUGAUGAG GCCGUUAGGC CGAA IGCUCCCA | 1147 |
| 348 | AUAUGUGC C CAAGAAUU | 243 | AAUUCUUG CUGAUGAG GCCGUUAGGC CGAA ICACAUAU | 1148 |
| 349 | UAUCUGCC C AAGAAUUU | 244 | AAAUUCUU CUGAUGAG GCCGUUAGGC CGAA IGCACAUA | 1149 |
| 350 | AUGUGCCC A AGAAUUUC | 245 | GAAAUUCU CUGAUGAG GCCGUUAGGC CGAA IGGCACAU | 1150 |
| 359 | AGAAUUUC A UAGACAUC | 246 | GAUGUCUA CUGAUGAG GCCGUUAGGC CGAA IAAAUUCU | 1151 |
| 365 | UCAUAGAC A UCCAGUUU | 247 | AAACUGGA CUGAUGAG GCCGUUAGGC CGAA IUCUAUGA | 1152 |
| 368 | UAGACAUC C AGUUUCCC | 248 | AGGAAACU CUGAUGAG GCCGUUAGGC CGAA IAUGUCUA | 1153 |
| 369 | AGACAUCC A GUUUCCCA | 249 | AGGGAAAC CUGAUGAG GCCGUUAGGC CGAA IGAUGUCU | 1154 |
| 375 | CCAGUUCC C CAAAUGGU | 250 | ACCAUUUG CUGAUGAG GCCGUUAGGC CGAA IAAACUGG | 1155 |
| 376 | CAGUUUCC C AAAUGGUU | 251 | AACCAUUU CUGAUGAG GCCGUUAGGC CGAA IGAAACUG | 1156 |
| 377 | AGUUUCCC A AAUGGUUU | 252 | AAACCAUU CUGAUGAG GCCGUUAGGC CGAA IGGAAACU | 1157 |
| 387 | AUGGUUUC A CGAAGGCC | 253 | GGCCUUCG CUGAUGAG GCCGUUAGGC CGAA IAAACCAU | 1158 |
| 395 | ACGAAGGC C UCUCUCGA | 254 | UCGAGAGA CUGAUGAG GCCGUUAGGC CGAA ICCUUCGU | 1159 |
| 396 | CGAAGGCC U CUCUCGAC | 255 | GUCGAGAG CUGAUGAG GCCGUUAGGC CGAA IGCCUUCG | 1160 |
| 398 | AAGGCCUC U CUCGACAC | 256 | GUGUCGAG CUGAUGAG GCCGUUAGGC CGAA IAGGCCUU | 1161 |
| 400 | GGCCUCUC U CGACACCA | 257 | UGGUGUCG CUGAUGAG GCCGUUAGGC CGAA IAGAGGCC | 1162 |
| 405 | CUCUCGAC A CCAGGCAG | 258 | CUGCCUGG CUGAUGAG GCCGUUAGGC CGAA IUCGAGAG | 1163 |
| 407 | CUCGACAC C AGGCAGAG | 259 | CUCUGCCU CUGAUGAG GCCGUUAGGC CGAA IUGUCGAG | 1164 |
| 408 | UCGACACC A GGCAGAGA | 260 | UCUCUGCC CUGAUGAG GCCGUUAGGC CGAA IGUGUCGA | 1165 |
| 412 | CACCAGGC A GAGAACUU | 261 | AAGUUCUC CUGAUGAG GCCGUUAGGC CGAA ICCUGGUG | 1166 |
| 419 | CAGAGAGC U UACUCAUG | 262 | CAUGAGUA CUGAUGAG GCCGUUAGGC CGAA IUUCUCUG | 1167 |
| 423 | GAACUUAC U CAUGGGCA | 263 | UGCCCAUG CUGAUGAG GCCGUUAGGC CGAA IUAAGUUC | 1168 |
| 425 | ACUUACUC A UGGGCAAG | 264 | CUUGCCCA CUGAUGAG GCCGUUAGGC CGAA IAGUAAGU | 1169 |
| 431 | UCAUGGGC A AGGAGGUU | 265 | AACCUCCU CUGAUGAG GCCGUUAGGC CGAA ICCCAUGA | 1170 |
| 443 | AGGUUGGC U UCUUCAUC | 266 | GAUGAAGA CUGAUGAG GCCGUUAGGC CGAA ICCAACCU | 1171 |
| 446 | UUGGCUUC U UCAUCAUC | 267 | GAUGAUGA CUGAUGAG GCCGUUAGGC CGAA IAAGCCAA | 1172 |
| 449 | GCUUCUUC A UCAUCCGG | 268 | CCGGAUGA CUGAUGAG GCCGUUAGGC CGAA IAAGAAGC | 1173 |
| 452 | UCUUCAUC A UCCGGGCC | 269 | GGCCCGGA CUGAUGAG GCCGUUAGGC CGAA IAUGAAGA | 1174 |
| 455 | UCAUCAUC C GGGCCAGC | 270 | GCUGGCCC CUGAUGAG GCCGUUAGGC CGAA IAUGAUGA | 1175 |
| 460 | AUCCGGGC C AGCCAGAG | 271 | CUCUGGCU CUGAUGAG GCCGUUAGGC CGAA ICCCGGAU | 1176 |
| 461 | UCCGGGCC A GCCAGAGC | 272 | GCUCUGGC CUGAUGAG GCCGUUAGGC CGAA IGCCCGGA | 1177 |
| 464 | GGGCCAGC C AGAGCUCC | 273 | GGAGCUCU CUGAUGAG GCCGUUAGGC CGAA ICUGGCCC | 1178 |
| 465 | GGCCAGCC A GAGCUCCC | 274 | GGGAGCUC CUGAUGAG GCCGUUAGGC CGAA IGCUGGCC | 1179 |
| 470 | GCCAGAUC U CCCCAGGG | 275 | CCCUGGGG CUGAUGAG GCCGUUAGGC CGAA ICUCUGGC | 1180 |
| 472 | CAGACCUC C CCAGGGGA | 276 | UCCCCUGG CUGAUGAG GCCGUUAGGC CGAA IAGCUCUG | 1181 |
| 473 | AGAGCUCC C CAGGGGAC | 277 | GUCCCCUG CUGAUGAG GCCGUUAGGC CGAA IGAGCUCU | 1182 |
| 474 | GAGCUCCC C AGGGGACU | 278 | AGUCCCCU CUGAUGAG GCCGUUAGGC CGAA IGGAGGUC | 1183 |
| 475 | AGCUCCCC A GGGGACUU | 279 | AAGUCCCC CUGAUGAG GCCGUUAGGC CGAA IGGGAGCU | 1184 |
| 482 | CAGGGGAC U UCUCCAUC | 280 | GAUGGAGA CUGAUGAG GCCGUUAGGC CGAA IUCCCCUG | 1185 |
| 485 | GGGACUUC U CCAUCUCU | 281 | AGAGAUGG CUGAUGAG GCCGUUAGGC CGAA IAAGUCCC | 1186 |
| 487 | GACCUCUC C AUCUCUGU | 282 | ACAGAGAU CUGAUGAG GCCGUUAGGC CGAA IAGAAGUC | 1187 |
| 488 | ACUUCUCC A UCUCUGUC | 283 | GACAGAGA CUGAUGAG GCCGUUAGGC CGAA IGAGAAGU | 1188 |
| 491 | UCUCCAUC U CUGUCAGG | 284 | CCUGACAG CUGAUGAG GCCGUUAGGC CGAA IAUGGAGA | 1189 |
| 493 | UCCAUCUC U GUCAGGCA | 285 | UGCCUGAC CUGAUGAG GCCGUUAGGC CGAA IAGAUGGA | 1190 |
| 497 | UCUCUGUC A GGCAUGAG | 286 | CUCAUGCC CUGAUGAG GCCGUUAGGC CGAA IACAGAGA | 1191 |
| 501 | UGUCAGGC A UGAGGAUG | 287 | CAUCCUCA CUGAUGAG GCCGUUAGGC CGAA ICCUGACA | 1192 |
| 516 | UGACGUUC A ACACUUCA | 288 | UGAAGUGU CUGAUGAG GCCGUUAGGC CGAA IAACGUCA | 1193 |
| 519 | CGUUCAAC A CUUCAAGG | 289 | ACUUGAAG CUGAUGAG GCCGUUAGGC CGAA IUUGAACG | 1194 |
| 521 | UUCAACAC U UCAAGGUC | 290 | GACCUUGA CUGAUGAG GCCGUUAGGC CGAA IUGUUGAA | 1195 |
| 524 | AACACUUC A AGGUCAUG | 291 | CAUGACCU CUGAUGAG GCCGUUAGGC CGAA IAAGUGUU | 1196 |
| 530 | UCAAGGUC A UGCGAGAC | 292 | GUCUCGCA CUGAUGAG GCCGUUAGGC CGAA IACCUUGA | 1197 |
| 539 | UGCGAGAC A ACAAGGGU | 293 | ACCCUUGU CUGAUGAG GCCGUUAGGC CGAA IUCUCGCA | 1198 |
| 542 | GAGACAAC A AGGGUAAU | 294 | AUUACCCU CUGAUGAG GCCGUUAGGC CGAA IUUGUCUC | 1199 |
| 554 | GUAAUUAC U UUCUGUGG | 295 | CCACAGAA CUGAUGAG GCCGUUAGGC CGAA IUAAUUAC | 1200 |
| 558 | UUACUUUC U GUGGACUG | 296 | CAGUCCAC CUGAUGAG GCCGUUAGGC CGAA IAAAGUAA | 1201 |
| 565 | CUGUGGAC U GAGAAGUU | 297 | AACUUCUC CUGAUGAG GCCGUUAGGC CGAA IUCCACAG | 1202 |
| 576 | GAAGUUUC C AUCCCUAA | 298 | UUAGGGAU CUGAUGAG GCCGUUAGGC CGAA IAAACUUC | 1203 |
| 577 | AAGUUUCC A UCCCUAAA | 299 | UUUAGGGA CUGAUGAG GCCGUUAGGC CGAA IGAAACUU | 1204 |
| 580 | UUUCCAUC C CUAAAUAA | 300 | UUAUUUAG CUGAUGAG GCCGUUAGGC CGAA IAUGGAAA | 1205 |
| 581 | UUCCAUCC C UAAAUAAG | 301 | CUUAUUUA CUGAUGAG GCCGUUAGGC CGAA IGAUGGAA | 1206 |
| 582 | UCCAUCCC U AAAUAAGC | 302 | GCUUAUUU CUGAUGAG GCCGUUAGGC CGAA IGGAUGGA | 1207 |
| 591 | AAAUAAGC U GGUAGACU | 303 | AGUCUACC CUGAUGAG GCCGUUAGGC CGAA ICUUAUUU | 1208 |
| 599 | UGGUAGAC U ACUACAGG | 304 | CCUGUAGU CUGAUGAG GCCGUUAGGC CGAA IUCUACCA | 1209 |
| 602 | UAGACUAC U ACAGGACA | 305 | UGUCCUGU CUGAUGAG GCCGUUAGGC CGAA IUAGUCUA | 1210 |
| 605 | ACUACUAC A GGACAAAU | 306 | AUUUGUCC CUGAUGAG GCCGUUAGGC CGAA IUAGUAGU | 1211 |
| 610 | UACAGGAC A AAUUCCAU | 307 | AUGGAAUU CUGAUGAG GCCGUUAGGC CGAA IUCCUGUA | 1212 |
| 616 | ACAAAUUC C AUCUCCAG | 308 | CUGGAGAC CUGAUGAG GCCGUUAGGC CGAA IAAUUUGU | 1213 |
| 617 | CAAAUUCC A UCUCCAGA | 309 | UCUGGAGA CUGAUGAG GCCGUUAGGC CGAA IGAAUUUG | 1214 |
| 620 | AUUCCAUC U CCAGACAG | 310 | CUGUCUGG CUGAUGAG GCCGUUAGGC CGAA IAUGGAAU | 1215 |
| 622 | UCCAUCUC C AGACAGAA | 311 | UUCUGUCU CUGAUGAG GCCGUUAGGC CGAA IAGAUGGA | 1216 |
| 623 | CCAUCUCC A GACAGAAG | 312 | CUUCUGUC CUGAUGAG GCCGUUAGGC CGAA IGAGAUGG | 1217 |
| 627 | CUCCAGAC A GAAGCAGA | 313 | UCUGCUUC CUGAUGAG GCCGUUAGGC CGAA IUCUGGAG | 1218 |
| 633 | ACAGAAGC A GAUCUUCC | 314 | GGAAGAUC CUGAUGAG GCCGUUAGGC CGAA ICUUCUGU | 1219 |
| 638 | AGCAGAUC U UCCUUAGA | 315 | UCUAAGGA CUGAUGAG GCCGUUAGGC CGAA IAUCUGCU | 1220 |
| 641 | AGAUCUUC C UUAGAGAC | 316 | GUCUCUAA CUGAUGAG GCCGUUAGGC CGAA IAAGAUCU | 1221 |
| 642 | GAUCUUCC U UAGAGACA | 317 | UGUCUCUA CUGAUGAG GCCGUUAGGC CGAA IGAAGAUC | 1222 |
| 650 | UUAGAGAC A GAACCCGA | 318 | UCGGGUUC CUGAUGAG GCCGUUAGGC CGAA IUCUCUAA | 1223 |
| 655 | GACAGAAC C CGAGAAGA | 319 | UCUUCUCG CUGAUGAG GCCGUUAGGC CGAA IUUCUGUC | 1224 |
| 656 | ACAGAACC C GAGAAGAC | 320 | GUCUUCUC CUGAUGAG GCCGUUAGGC CGAA IGUUCUGU | 1225 |
| 665 | GAGAAGAC C AGGGUCAC | 321 | GUGACCCU CUGAUGAG GCCGUUAGGC CGAA IUCUUCUC | 1226 |
| 666 | AGAAGACC A GGGUCACC | 322 | GGUGACCC CUGAUGAG GCCGUUAGGC CGAA IGUCUUCU | 1227 |
| 672 | CCAGGGUC A CCGGGGCA | 323 | UGCCCCGG CUGAUGAG GCCGUUAGGC CGAA IACCCUGG | 1228 |
| 674 | AGGGUCAC C GGGGCAAC | 324 | GUUGCCCC CUGAUGAG GCCGUUAGGC CGAA IUGACCCU | 1229 |
| 680 | ACCGGGGC A ACAGCCUG | 325 | CAGGCUGU CUGAUGAG GCCGUUAGGC CGAA ICCCCGGU | 1230 |
| 683 | GGGGCAAC A GCCUGGAC | 326 | GUCCAGGC CUGAUGAG GCCGUUAGGC CGAA IUUGCCCC | 1231 |
| 686 | GCAACAGC C UGGACCGG | 327 | CCGGUCCA CUGAUGAG GCCGUUAGGC CGAA ICUGUUGC | 1232 |
| 687 | CAACAGCC U GGACCGGA | 328 | UCCGGUCC CUGAUGAG GCCGUUAGGC CGAA IGCUGUUG | 1233 |
| 692 | GCCUGGAC C GGAGGUCC | 329 | GGACCUCC CUGAUGAG GCCGUUAGGC CGAA IUCCAGGC | 1234 |
| 700 | CGGAGGUC C CAGGGAGG | 330 | CCUCCCUG CUGAUGAG GCCGUUAGGC CGAA IACCUCCG | 1235 |
| 701 | GGAGGUCC C AGGGAGGC | 331 | GCCUCCCU CUGAUGAG GCCGUUAGGC CGAA IGACCUCC | 1236 |
| 702 | GAGGUCCC A GGGAGGCC | 332 | GGCCUCCC CUGAUGAG GCCGUUAGGC CGAA IGGACCUC | 1237 |
| 710 | AGGGAGGC C CACACCUC | 333 | GAGGUGUG CUGAUGAG GCCGUUAGGC CGAA ICCUCCCU | 1238 |
| 711 | GGGAGGCC C ACACCUCA | 334 | UGAGGUGU CUGAUGAG GCCGUUAGGC CGAA IGCCUCCC | 1239 |
| 712 | GGAGGCCC A CACCUCAG | 335 | CUGAGGUG CUGAUGAG GCCGUUAGGC CGAA IGGCCUCC | 1240 |
| 714 | AGGCCCAC A CCUCAGUG | 336 | CACUGAGG CUGAUGAG GCCGUUAGGC CGAA IUGGGCCU | 1241 |
| 716 | GCCCACAC C UCAGUGGG | 337 | CCCACUGA CUGAUGAG GCCGUUAGGC CGAA IUGUGGGC | 1242 |
| 717 | CCCACACC U CAGUGGGG | 338 | CCCCACUG CUGAUGAG GCCGUUAGGC CGAA IGUGUGGG | 1243 |
| 719 | CACACCUC A GUGGGGCU | 339 | AGCCCCAC CUGAUGAG GCCGUUAGGC CGAA IAGGUGUG | 1244 |
| 727 | AGUGGGGC U GUGGGAGA | 340 | UCUCCCAC CUGAUGAG GCCGUUAGGC CGAA ICCCCACU | 1245 |
| 743 | AAGAAAUC C GACCUUCG | 341 | CGAAGGUC CUGAUGAG GCCGUUAGGC CGAA IAUUUCUU | 1246 |
| 747 | AAUCCGAC C UUCGAUGA | 342 | UCAUCGAA CUGAUGAG GCCGUUAGGC CGAA IUCGGAUU | 1247 |
| 748 | AUCCGACC U UCGAUGAA | 343 | UUCAUCGA CUGAUGAG GCCGUUAGGC CGAA IGUCGGAU | 1248 |
| 758 | CGAUGAAC C GGAAGCUG | 344 | CAGCUUCC CUGAUGAG GCCGUUAGGC CGAA IUUCAUCG | 1249 |
| 765 | CCGGAAGC U GUCGGAUC | 345 | GAUCCGAC CUGAUGAG GCCGUUAGGC CGAA ICUUCCGG | 1250 |
| 774 | GUCGGAUC A CCCCCCGA | 346 | UCGGGGGG CUGAUGAG GCCGUUAGGC CGAA IAUCCGAC | 1251 |
| 776 | CGGAUCAC C CCCCGACC | 347 | GGUCGGGG CUGAUGAG GCCGUUAGGC CGAA IUGAUCCG | 1252 |
| 777 | GGAUCACC C CCCGACCC | 348 | GGGUCGGG CUGAUGAG GCCGUUAGGC CGAA IGUGAUCC | 1253 |
| 778 | GAUCACCC C CCGACCCU | 349 | AGGGUCGG CUGAUGAG GCCGUUAGGC CGAA IGGUGAUC | 1254 |
| 779 | AUCACCCC C CGACCCUU | 350 | AAGGGUCG CUGAUGAG GCCGUUAGGC CGAA IGGGUGAU | 1255 |
| 780 | UCACCCCC C GACCCUUC | 351 | GAAGGGUC CUGAUGAG GCCGUUAGGC CGAA IGGGGUGA | 1256 |
| 784 | CCCCCGAC C CUUCCCCU | 352 | AGGGGAAG CUGAUGAG GCCGUUAGGC CGAA IUCGGGGG | 1257 |
| 785 | CCCCGACC C UUCCCCUG | 353 | CAGGGGAA CUGAUGAG GCCGUUAGGC CGAA IGUCGGGG | 1258 |
| 786 | CCCGACCC U UCCCCUGC | 354 | GCAGGGGA CUGAUGAG GCCGUUAGGC CGAA IGGUCGGG | 1259 |
| 789 | GACCCUUC C CCUGCAGC | 355 | GCUGCAGG CUGAUGAG GCCGUUAGGC CGAA IAAGGGUC | 1260 |
| 790 | ACCCUUCC C CUGCAGCA | 356 | UGCUGCAG CUGAUGAG GCCGUUAGGC CGAA IGAAGGGU | 1261 |
| 791 | CCCUUCCC C UGCAGCAG | 357 | CUGCUGCA CUGAUGAG GCCGUUAGGC CGAA IGGAAGGG | 1262 |
| 792 | CCUUCCCC U GCAGCAGC | 358 | GCUGCUGC CUGAUGAG GCCGUUAGGC CGAA IGGGAAGG | 1263 |
| 795 | UCCCCUGC A GCAGCACC | 359 | GGUGCUGC CUGAUGAG GCCGUUAGGC CGAA ICAGGGGA | 1264 |
| 798 | CCUGCAGC A GCACCAGC | 360 | GCUGGUGC CUGAUGAG GCCGUUAGGC CGAA ICUGCAGG | 1265 |
| 801 | GCAGCAGC A CCAGCACC | 361 | GGUGCUGG CUGAUGAG GCCGUUAGGC CGAA ICUGCUGC | 1266 |
| 803 | AGCAGCAC C AGCACCAG | 362 | CUGGUGCU CUGAUGAG GCCGUUAGGC CGAA IUGCUGCU | 1267 |
| 804 | GCACCACC A GCACCAGC | 363 | GCUGGUGC CUGAUGAG GCCGUUAGGC CGAA IGUGCUGC | 1268 |
| 807 | GCACCAGC A CCAGCCAC | 364 | GUGGCUGG CUGAUGAG GCCGUUAGGC CGAA ICUGGUGC | 1269 |
| 809 | ACCACCAC C AGCCACAG | 365 | CUGUGGCU CUGAUGAG GCCGUUAGGC CGAA IUGCUGGU | 1270 |
| 810 | CCAGCACC A GCCACAGC | 366 | GCUGUGGC CUGAUGAG GCCGUUAGGC CGAA IGUGCUGG | 1271 |
| 813 | GCACCAGC C ACAGCCUC | 367 | GAGGCUGU CUGAUGAG GCCGUUAGGC CGAA ICUGGUGC | 1272 |
| 814 | CACCAGCC A CAGCCUCC | 368 | GGAGGCUG CUGAUGAG GCCGUUAGGC CGAA IGCUGGUG | 1273 |
| 816 | CCAGCCAC A GCCUCCGC | 369 | GCGGAGGC CUGAUGAG GCCGUUAGGC CGAA IUGGCUGG | 1274 |
| 819 | GCCACAGC C UCCGCAAU | 370 | AUUGCGGA CUGAUGAG GCCGUUAGGC CGAA ICUGUGGC | 1275 |
| 820 | CCACAGCC U CCGCAAUA | 371 | UAUUGCGG CUGAUGAG GCCGUUAGGC CGAA IGCUGUGG | 1276 |
| 822 | ACAGCCUC C GCAAUAUG | 372 | CAUAUUGC CUGAUGAG GCCGUUAGGC CGAA IAGGCUGU | 1277 |
| 825 | GCCUCCGC A AUAUGCCC | 373 | GGGCAUAU CUGAUGAG GCCGUUAGGC CGAA ICGGAGGC | 1278 |
| 832 | CAAUAUGC C CCAGCGCC | 374 | GGCGCUGG CUGAUGAG GCCGUUAGGC CGAA ICAGACUG | 1279 |
| 833 | AAUAUGCC C CAGCGCCC | 375 | GGGCGCUG CUGAUGAG GCCGUUAGGC CGAA IGCAUAUU | 1280 |
| 834 | AUAUGCCC C AGCGCCCC | 376 | GGGGCGCU CUGAUGAG GCCGUUAGGC CGAA IGGCAUAU | 1281 |
| 835 | UAUGCCCC A GCGCCCCA | 377 | UGGGGCGC CUGAUGAG GCCGUUAGGC CGAA IGGGCAUA | 1282 |
| 840 | CCCAGCGC C CCAGCAGC | 378 | GCUGCUGG CUGAUGAG GCCGUUAGGC CGAA ICGCUGGG | 1283 |
| 841 | CCAGCGCC C CAGCAGCU | 379 | AGCUGCUG CUGAUGAG GCCGUUAGGC CGAA IGCGCUGG | 1284 |
| 842 | CAGCGCCC C AGCAGCUG | 380 | CAGCUGCU CUGAUGAG GCCGUUAGGC CGAA IGGCGCUG | 1285 |
| 843 | AGCGCCCC A GCAGCUGC | 381 | GCAGCUGC CUGAUGAG GCCGUUAGGC CGAA IGGGCGCU | 1286 |
| 846 | GCCCCAGC A GCUGCAGC | 382 | GCUGCAGC CUGAUGAG GCCGUUAGGC CGAA ICUGGGGC | 1287 |
| 849 | CCAGCAGC U GCAGCAGC | 383 | GCUGCUGC CUGAUGAG GCCGUUAGGC CGAA ICUGCUGG | 1288 |
| 852 | GCAGCUGC A GCAGCCCC | 384 | GGGGCUGC CUGAUGAG GCCGUUAGGC CGAA ICAGCUGC | 1289 |
| 855 | GCUGCAGC A GCCCCCAC | 385 | GUGGGGGC CUGAUGAG GCCGUUAGGC CGAA ICUGCAGC | 1290 |
| 858 | GCAGCAGC C CCCACAGC | 386 | GCUGUGGG CUGAUGAG GCCGUUAGGC CGAA ICUGCUGC | 1291 |
| 859 | CAGCAGCC C CCACAGCA | 387 | UGCUGUGG CUGAUGAG GCCGUUAGGC CGAA IGCUGCUG | 1292 |
| 860 | AGCAGCCC C CACAGCAG | 388 | CUGCUGUG CUGAUGAG GCCGUUAGGC CGAA IGGCUGCU | 1293 |
| 861 | GCAGCCCC C ACAGCAGC | 389 | GCUGCUGU CUGAUGAG GCCGUUAGGC CGAA IGGGCUGC | 1294 |
| 862 | CAGCCCCC A CAGCAGCG | 390 | CGCUGCUG CUGAUGAG GCCGUUAGGC CGAA IGGGGCUG | 1295 |
| 864 | GCCCCCAC A GCAGCGAU | 391 | AUCGCUGC CUGAUGAG GCCGUUAGGC CGAA IUGGGGGC | 1296 |
| 867 | CCCACAGC A GCGAUAUC | 392 | GAUAUCGC CUGAUGAG GCCGUUAGGC CGAA ICUGUGGG | 1297 |
| 876 | GCGAUAUC U GCAGCACC | 393 | GGUGCUGC CUGAUGAG GCCGUUAGGC CGAA IAUAUCGC | 1298 |
| 879 | AUAUCUGC A GCACCACC | 394 | GGUGGUGC CUGAUGAG GCCGUUAGGC CGAA ICAGAUAU | 1299 |
| 882 | UCUGCAGC A CCACCAUU | 395 | AAUGGUGG CUGAUGAG GCCGUUAGGC CGAA ICUGCAGA | 1300 |
| 884 | UGCAGCAC C ACCAUCUC | 396 | GAAAUGGU CUGAUGAG GCCGUUAGGC CGAA IUGCUGCA | 1301 |
| 885 | GCAGCACC A CCAUUUCC | 397 | GGAAAUGG CUGAUGAG GCCGUUAGGC CGAA IGUGCUGC | 1302 |
| 887 | AGCACCAC C AUCUCCAC | 398 | GUGGAAAU CUGAUGAG GCCGUUAGGC CGAA IUGGUGCU | 1303 |
| 888 | GCACCACC A UUUCCACC | 399 | GGUGGAAA CUGAUGAG GCCGUUAGGC CGAA IGUGGUGC | 1304 |
| 893 | ACCAUCUC C ACCAGGAA | 400 | UUCCUGGU CUGAUGAG GCCGUUAGGC CGAA IAAAUGGU | 1305 |
| 894 | CCAUUUCC A CCAGGAAC | 401 | GUUCCUGG CUGAUGAG GCCGUUAGGC CGAA IGAAAUGG | 1306 |
| 896 | AUCUCCAC C AGGAACGC | 402 | GCGUUCCU CUGAUGAG GCCGUUAGGC CGAA IUGGAAAU | 1307 |
| 897 | UUUCCACC A GGAACGCC | 403 | GGCGUUCC CUGAUGAG GCCGUUAGGC CGAA IGUGGAAA | 1308 |
| 905 | AGGAACGC C GAGGAGGC | 404 | GCCUCCUC CUGAUGAG GCCGUUAGGC CGAA ICGUUCCU | 1309 |
| 914 | GAGGAGUC A GCCUUGAC | 405 | GUCAAGGC CUGAUGAG GCCGUUAGGC CGAA ICCUCCUC | 1310 |
| 917 | GAGGCAGC C UUGACAUA | 406 | UAUGUCAA CUGAUGAG GCCGUUAGGC CGAA ICUGCCUC | 1311 |
| 918 | AGGCAGCC U UGACAUAA | 407 | UCAUGUCA CUGAUGAG GCCGUUAGGC CGAA IGCUGCCU | 1312 |
| 923 | GCCUUGAC A UAAAUGAU | 408 | AUCAUUUA CUGAUGAG GCCGUUAGGC CGAA IUCAAGGC | 1313 |
| 936 | UGAUGGGC A UUGUGGCA | 409 | UGCCACAA CUGAUGAG GCCGUUAGGC CGAA ICCCAUCA | 1314 |
| 944 | AUUGUGGC A CCGGCUUG | 410 | CAAGCCGG CUGAUGAG GCCGUUAGGC CGAA ICCACAAU | 1315 |
| 946 | UGUGGCAC C GGCUUGGG | 411 | CCCAAGCC CUGAUGAG GCCGUUAGGC CGAA IUGCCACA | 1316 |
| 950 | GCACCGGC U UGGGCAGU | 412 | ACUGCCCA CUGAUGAG GCCGUUAGGC CGAA ICCGGUGC | 1317 |
| 956 | GCUUGGGC A GUGAAAUG | 413 | CACUCCAC CUGAUGAG GCCGUUAGGC CGAA ICCCAAGC | 1318 |
| 973 | AAUGCGGC C CUCAUGCA | 414 | UGCAUGAG CUGAUGAG GCCGUUAGGC CGAA ICCUCAUC | 1319 |
| 974 | AUGCGGCC C UCAUGCAU | 415 | AUGCAUGA CUGAUGAG GCCGUUAGGC CGAA IGCCGCAU | 1320 |
| 975 | UGCGGCCC U CAUGCAUC | 416 | GAUGCAUG CUGAUGAG GCCGUUAGGC CGAA IGGCCGCA | 1321 |
| 977 | CGGCCCUC A UGCAUCGG | 417 | CCGAUGCA CUGAUGAG GCCGUUAGGC CGAA IAGGGCCG | 1322 |
| 981 | CCUCAUGC A UCGGAGAC | 418 | GUCUCCGA CUGAUGAG GCCGUUAGGC CGAA ICAUGAGG | 1323 |
| 990 | UCGGAGAC A CACAGACC | 419 | GGUCUGUG CUGAUGAG GCCGUUAGGC CGAA IUCUCCGA | 1324 |
| 992 | GGAGACAC A CAGACCCA | 420 | UGGGUCUG CUGAUGAG GCCGUUAGGC CGAA IUGUCUCC | 1325 |
| 994 | AGACACAC A GACCCAGU | 421 | ACUGGGUC CUGAUGAG GCCGUUAGGC CGAA IUGUGUCU | 1326 |
| 998 | ACACAGAC C CAGUCCAG | 422 | CUGCACUG CUGAUGAG GCCGUUAGGC CGAA IUCUGUGU | 1327 |
| 999 | CACAGAUC C AGUGCAGC | 423 | GCUGCACU CUGAUGAG GCCGUUAGGC CGAA IGUCUGUG | 1328 |
| 1000 | ACAGACCC A GUGCAGCU | 424 | AGCUGCAC CUGAUGAG GCCGUUAGGC CGAA IGGUCUGU | 1329 |
| 1005 | CCCAGUGC A GCUCCAGG | 425 | CCUGGAGC CUGAUGAG GCCGUUAGGC CGAA ICACUGGG | 1330 |
| 1008 | AGUGCAGC U CCAGGCGG | 426 | CCGCCUGG CUGAUGAG GCCGUUAGGC CGAA ICUGCACU | 1331 |
| 1010 | UGCAGCUC C AGGCGGCA | 427 | UGCCGCCU CUGAUGAG GCCGUUAGGC CGAA IAGCUGCA | 1332 |
| 1011 | GCAGCUCC A GGCGGCAG | 428 | CUGCCGCC CUGAUGAG GCCGUUAGGC CGAA IGAGCUGC | 1333 |
| 1018 | CAGGUGGC A GGGCGAGU | 429 | ACUCGCCC CUGAUGAG GCCGUUAGGC CGAA ICCGCCUG | 1334 |
| 1036 | CUGUGGUC C CGGGCGCU | 430 | AGCGCCCG CUGAUGAG GCCGUUAGGC CGAA ICCCACCG | 1335 |
| 1037 | GGUGGGCC C GGGCGCUG | 431 | CAGCGCCC CUGAUGAG GCCGUUAGGC CGAA IGCCCACC | 1336 |
| 1044 | CCGGGCGC U GUAUGACU | 432 | AGUCAUAC CUGAUGAG GCCGUUAGGC CGAA ICGCCCGG | 1337 |
| 1052 | UGUAUGAC U UUGAGGCC | 433 | GGCCUCAA CUGAUGAG GCCGUUAGGC CGAA IUCAUACA | 1338 |
| 1060 | UUUGAGGC C CUGGAGGA | 434 | UCCUCCAG CUGAUGAG GCCGUUAGGC CGAA ICCUCAAA | 1339 |
| 1061 | UUGAGGCC C UGGAGGAU | 435 | AUCCUCCA CUGAUGAG GCCGUUAGGC CGAA IGCCUCAA | 1340 |
| 1062 | UGAGGCCC U GGAGGAUG | 436 | CAUCCUCC CUGAUGAG GCCGUUAGGC CGAA IGGCCUCA | 1341 |
| 1077 | UGACGAGC U GGGGUUCC | 437 | GGAACCCC CUGAUGAG GCCGUUAGGC CGAA ICUCGUCA | 1342 |
| 1085 | UGGGGUUC C ACAGCGGG | 438 | CCCGCUGU CUGAUGAG GCCGUUAGGC CGAA IAACCCCA | 1343 |
| 1086 | GGGGUUCC A CAGCGGGG | 439 | CCCCGCUG CUGAUGAG GCCGUUAGGC CGAA IGAACCCC | 1344 |
| 1088 | GGUUCCAC A GCGGGGAG | 440 | CUCCCCGC CUGAUGAG GCCGUUAGGC CGAA IUGGAACC | 1345 |
| 1109 | UGGAGGUC C UGGAUAGC | 441 | GCUAUCCA CUGAUGAG GCCGUUAGGC CGAA IACCUCCA | 1346 |
| 1110 | GGAGGUCC U GGAUAGCU | 442 | AGCUAUCC CUGAUGAG GCCGUUAGGC CGAA IGACCUCC | 1347 |
| 1118 | UGGAUAGC U CCAACCCA | 443 | UGGGUUGG CUGAUGAG GCCGUUAGGC CGAA ICUAUCCA | 1348 |
| 1120 | GAUAGCUC C AACCCAUC | 444 | GAUGGGUU CUGAUGAG GCCGUUAGGC CGAA IAGCUAUC | 1349 |
| 1121 | AUAGCUCC A ACCCAUCC | 445 | GGAUGGGU CUGAUGAG GCCGUUAGGC CGAA IGAGCUAU | 1350 |
| 1124 | GCUCCAAC C CAUCCUGG | 446 | CCAGGAUG CUGAUGAG GCCGUUAGGC CGAA IUUGGAGC | 1351 |
| 1125 | CUCCAACC C AUCCUGGU | 447 | ACCAGGAU CUGAUGAG GCCGUUAGGC CGAA IGUUGGAG | 1352 |
| 1126 | UCCAACCC A UCCUGGUG | 448 | CACCAGGA CUGAUGAG GCCGUUAGGC CGAA IGGUUGGA | 1353 |
| 1129 | AACCCAUC C UGGUGGAC | 449 | GUCCACCA CUGAUGAG GCCGUUAGGC CGAA IAUGGGUU | 1354 |
| 1130 | ACCCAUCC U GGUGGACC | 450 | GGUCCACC CUGAUGAG GCCGUUAGGC CGAA IGAUGGGU | 1355 |
| 1138 | UGGUGGAC C GGCCGCCU | 451 | AGGCGGCC CUGAUGAG GCCGUUAGGC CGAA IUCCACCA | 1356 |
| 1142 | GGACCGGC C GCCUGCAC | 452 | GUGCAGGC CUGAUGAG GCCGUUAGGC CGAA ICCGGUCC | 1357 |
| 1145 | CCGGCCGC C UGCACAAC | 453 | GUUGUGCA CUGAUGAG GCCGUUAGGC CGAA ICGGCCGG | 1358 |
| 1146 | CGGCCGCC U GCACAACA | 454 | UGUUGUGC CUGAUGAG GCCGUUAGGC CGAA IGCGGCCG | 1359 |
| 1149 | CCGCCUGC A CAACAAGC | 455 | GCUUGUUG CUGAUGAG GCCGUUAGGC CGAA ICAGGCGG | 1360 |
| 1151 | GCCUGCAC A ACAAGCUG | 456 | CAGCUUGU CUGAUGAG GCCGUUAGGC CGAA IUGCAGGC | 1361 |
| 1154 | UGCACAAC A AGCUGGGC | 457 | GCCCAGCU CUGAUGAG GCCGUUAGGC CGAA IUUGUGCA | 1362 |
| 1158 | CAACAAGC U GGGCCUCU | 458 | AGAGGCCC CUGAUGAG GCCGUUAGGC CGAA ICUUGUUG | 1363 |
| 1163 | AGCUGGGC C UCUUCCCU | 459 | AGGGAAGA CUGAUGAG GCCGUUAGGC CGAA ICCCAGCU | 1364 |
| 1164 | GCUGGGCC U CUUCCCUG | 460 | CAGGGAAG CUGAUGAG GCCGUUAGGC CGAA IGCCCAGC | 1365 |
| 1166 | UGGGCCUC U UCCCUGCC | 461 | GGCAGGGA CUGAUGAG GCCGUUAGGC CGAA IAGGCCCA | 1366 |
| 1169 | GCCUCUUC C CUGCCAAC | 462 | GUUGGCAG CUGAUGAG GCCGUUAGGC CGAA IAAGAGGC | 1367 |
| 1170 | CCUCUUCC C UGCCAACU | 463 | AGUUGGCA CUGAUGAG GCCGUUAGGC CGAA IGAAGAGG | 1368 |
| 1171 | CUCUUCCC U GCCAACUA | 464 | UAGUUGGC CUGAUGAG GCCGUUAGGC CGAA IGGAAGAG | 1369 |
| 1174 | UUCCCUGC C AACUACGU | 465 | ACGUAGUU CUGAUGAG GCCGUUAGGC CGAA ICAGGGAA | 1370 |
| 1175 | UCCCUGCC A ACUACGUG | 466 | CACGUAGU CUGAUGAG GCCGUUAGGC CGAA IGCAGGGA | 1371 |
| 1178 | CUGCCAAC U ACGUGGCA | 467 | UGCCACGU CUGAUGAG GCCGUUAGGC CGAA IUUGGCAG | 1372 |
| 1186 | UACGUGGC A CCCAUGAC | 468 | GUCAUGGG CUGAUGAG GCCGUUAGGC CGAA ICCACGUA | 1373 |
| 1188 | CGUGGCAC C CAUGACCC | 469 | GGGUCAUG CUGAUGAG GCCGUUAGGC CGAA IUGCCACG | 1374 |
| 1189 | GUGGCACC C AUGACCCG | 470 | CGGGUCAU CUGAUGAG GCCGUUAGGC CGAA IGUGCCAC | 1375 |
| 1190 | UGGCACCC A UGACCCGA | 471 | UCGGGUCA CUGAUGAG GCCGUUAGGC CGAA IGGUGCCA | 1376 |
| 1195 | CCCAUGAC C CGAUAAAC | 472 | GUUUAUCG CUGAUGAG GCCGUUAGGC CGAA IUCAUGGG | 1377 |
| 1196 | CCAUGACC C GAUAAACU | 473 | AGUUUAUC CUGAUGAG GCCGUUAGGC CGAA IGUCAUGG | 1378 |
| 1204 | CGAUAAAC U CUUCAGGG | 474 | CCCUGAAG CUGAUGAG GCCGUUAGGC CGAA IUUUAUCG | 1379 |
| 1206 | AUAAACUC U UCAGGGGA | 475 | UCCCCUGA CUGAUGAG GCCGUUAGGC CGAA IAGUUUAU | 1380 |
| 1209 | AACUCUUC A GGGGACAG | 476 | CUGUCCCC CUGAUGAG GCCGUUAGGC CGAA IAAGAGUU | 1381 |
| 1216 | CAGGGGAC A GAAGCUUU | 477 | AAAGCUUC CUGAUGAG GCCGUUAGGC CGAA IUCCCCUG | 1382 |
| 1222 | ACAGAAGC U UUUUGUCU | 478 | AGACAAAA CUGAUGAG GCCGUUAGGC CGAA ICUUCUGU | 1383 |
| 1230 | UUUUUGUC U GGAGCUGC | 479 | GCAGCUCC CUGAUGAG GCCGUUAGGC CGAA IACAAAAA | 1384 |
| 1236 | UCUGGAGC U GCCCACAA | 490 | UUGUGGGC CUGAUGAG GCCGUUAGGC CGAA ICUCCAGA | 1385 |
| 1239 | GGAGCUGC C CACAAGAA | 481 | UUCUUGUG CUGAUGAG GCCGUUAGGC CGAA ICAGCUCC | 1386 |
| 1240 | GAGCUGCC C ACAAGAAA | 482 | UUUCUUGU CUGAUGAG GCCGUUAGGC CGAA IGCAGCUC | 1387 |
| 1241 | AGCUGCCC A CAAGAAAG | 483 | CUUUCUUG CUGAUGAG GCCGUUAGGC CGAA IGGCAGCU | 1388 |
| 1243 | CUGCCCAC A AGAAAGAG | 484 | CUCUUUCU CUGAUGAG GCCGUUAGGC CGAA IUGGGCAG | 1389 |
| 1255 | AAGAGGGC A AGGAAAAA | 485 | UUUUUCCU CUGAUGAG GCCGUUAGGC CGAA ICCCUCUU | 1390 |
| 1268 | AAAAAGGC U GGACUCCA | 486 | UGGAGUCC CUGAUGAG GCCGUUAGGC CGAA ICCUUUUU | 1391 |
| 1273 | GGCUGGAC U CCAUGACU | 487 | AGUCAUGG CUGAUGAG GCCGUUAGGC CGAA IUCCAGCC | 1392 |
| 1275 | CUGGACUC C AUGACUAU | 488 | AUAGUCAU CUGAUGAG GCCGUUAGGC CGAA IAGUCCAG | 1393 |
| 1276 | UGGACUCC A UGACUAUA | 489 | UAUAGUCA CUGAUGAG GCCGUUAGGC CGAA IGAGUCCA | 1394 |
| 1281 | UCCAUGAC U AUAUAUAC | 490 | GUAUAUAU CUGAUGAG GCCGUUAGGC CSAA IUCAUGGA | 1395 |
| 1290 | AUAUAUAC A UACAUCUA | 491 | UAGAUGUA CUGAUGAG GCCGUUAGGC CGAA IUAUAUAU | 1396 |
| 1294 | AUACAUAC A UCUAUCUA | 492 | UAGAUAGA CUGAUGAG GCCGUUAGGC CGAA IUAUGUAU | 1397 |

Input Sequence = HSA011736.
Cut Site = CH/.
Stem Length = 8.
Core Sequence = CUGAUGAG GCCGUUAGGC CGAA
HSA011736 (Homo sapiens mRNA for growth factor receptor binding protein (GRBLG); 1303 bp)
Underlined region can be any X sequence or linker as defined herein.
I = Inosine

TABLE V

Human GRID G-cleaver Ribozyme and Substrate Sequence

| | | | | |
|---|---|---|---|---|
| Pos | Substrate | Seq ID | Ribozyme | Seq ID |

| | | | | |
|---|---|---|---|---|
| 31 | GUAAACUU G CACCCUCU | 493 | AGAGGGUG UGAUG GCAUGCACUAUGC GCG AAGUUUAC | 1398 |
| 85 | CAUACUCU G AAAUGCAG | 494 | CUGCAUUU UGAUG GCAUGCACUAUGC GCG AGAGUAUG | 1399 |
| 90 | UCUGAAAU G CAGUAACU | 495 | AGUUACUG UGAUG GCAUGCACUAUGC GCG AUUUCAGA | 1400 |
| 101 | GUAACUCU G AUGCUUGA | 496 | UCAAGCAU UGAUG GCAUGCACUAUGC GCG AGAGGUAC | 1401 |
| 104 | ACUCUGAU G CUUGAAUU | 497 | AAUUCAAG UGAUG GCAUGCACUAUGC GCG AUCAGAGU | 1402 |
| 108 | UGAUGCUU G AAUUUGUU | 498 | AACAAAUU UGAUG GCAUGCACUAUGC GCG AAGCAUCA | 1403 |
| 127 | CCCUUCUU G CCAGAAAG | 499 | CUUUCUGG UGAUG GCAUGCACUAUGC GCG AAGAAGGG | 1404 |
| 221 | AAGCUGUU G CCAAGUUU | 500 | AAACUUGG UGAUG GCAUGCACUAUGC GCG AACAGCUU | 1405 |
| 230 | CCAAGUUU G AUUUCACU | 501 | AGUGAAAU UGAUG GCAUGCACUAUGC GCG AAACUUGG | 1406 |
| 239 | AUUUCACU G CUUCAGGU | 502 | ACCUGAAG UGAUG GCAUGCACUAUGC GCG AGUGAAAU | 1407 |
| 248 | CUUCAGGU G AGGAUGAA | 503 | UUCAUCCU UCAUG GCAUGCACUAUGC GCG ACCUGAAG | 1408 |
| 254 | GUGAGGAU G AACUGAGC | 504 | GCUCAGUU UGAUG GCAUGCACUAUGC GCG AUCCUCAC | 1409 |
| 259 | GAUGAACU G AGCUUUCA | 505 | UGAAAGCU UGAUG GCAUGCACUAUGC GCG AGUUCAUC | 1410 |
| 283 | GAUGUUUU G AAGAUUUU | 506 | AAAAUCUU UGAUG GCAUGCACUAUGC GCG AAAACAUC | 1411 |
| 346 | GGAUAUGU G CCCAAGAA | 507 | UUCUUGGG UGAUG GCAUGCACUAUGC GCG ACAUAUCC | 1412 |
| 389 | GGUUUCAC G AAGGCCUC | 508 | CAGGCCUU UGAUG GCAUGCACUAUGC GCG GUGAAACC | 1413 |
| 402 | CCUCUCUC G ACACCAGG | 509 | CCUGGUGU UGAUG GCAUGCACUAUGC GCG GAGAGAGG | 1414 |
| 503 | UCAGGCAU G AGGAUGAC | 510 | GUCAUCCU UGAUG GCAUGCACUAUGC GCG AUGUCUGA | 1415 |
| 509 | AUGAGGAU G ACGUUCAA | 511 | UUGAACGU UGAUG GCAUGCACUAUGC GCG AUCCUCAU | 1416 |
| 532 | AAGGUCAU G CGAGACAA | 512 | UUGUCUCG UCAUG GCAUGCACUAUGC GCC AUGACCUU | 1417 |
| 534 | GGUCAUGC G AGACAACA | 513 | UGUUGUCU UCAUG GCAUGCACUAUGC GCG GCAUGACC | 1418 |
| 566 | UGUGGACU G AGAAGUUU | 514 | AAACUUCU UGAUG GCAUGCACUAUGC GCG AGUCCACA | 1419 |
| 657 | CAGAACCC G AGAAGACC | 515 | GGUCUUCU UGAUG GCAUGCACUAUGC GCG GGGUUCUG | 1420 |
| 744 | AGAAAUCC G ACCUUCGA | 516 | UCGAAGGU UGAUG GCAUGCACUAUGC GCG GGAUUUCU | 1421 |
| 751 | CGACCUUC G AUGAACCG | 517 | CGGUUCAU UGAUG GCAUGCACUAUGC GCG GAAGGUCG | 1422 |
| 754 | CCUUCGAU G AACCGGAA | 518 | UUCCGGUU UGAUG GCAUGCACUAUGC GCG AUCGAAGG | 1423 |
| 781 | CACCCCCC G ACCCUUCC | 519 | GGAAGGGU UGAUG GCAUGCACUAUGC GCG GGGGGGUG | 1424 |
| 793 | CUUCCCCU G CAGCAGCA | 520 | UGCUGCUG UGAUG GCAUGCACUAUGC GCG AGGGGAAG | 1425 |
| 823 | CAGCCUCC G CAAUAUGC | 521 | GCAUAUUG UCAUG GCAUGCACUAUGC GCG GGAGGCUG | 1426 |
| 830 | CGCAAUAU G CCCCAGCG | 522 | CGCUGGGG UGAUG GCAUGCACUAUGC GCG AUAUUGCG | 1427 |
| 838 | GCCCCAGC G CCCCAGCA | 523 | UGCUGGGG UCAUG GCAUGCACUAUGC GCG GCUGGGGC | 1428 |
| 850 | CAGCAGUC G CAGCAGCC | 524 | GGCUGCUG UGAUG GCAUGCACUAUGC GCG AGCUGCUG | 1429 |
| 870 | ACAGCAGC G AUAUCUGC | 525 | GCAGAUAU UCAUG GCAUGCACUAUGC GCG GCUGCUGU | 1430 |
| 877 | CGAUAUCU G CAGCACCA | 526 | UGGUGCUG UGAUG GCAUGCACUAUGC GCG AGAUAUCG | 1431 |
| 903 | CCAGGAAC G CCGAGGAG | 527 | CUCCUCGG UGAUG GCAUGCACUAUGC GCG GUUCCUGG | 1432 |
| 906 | GGAACGCC G AGGAGGCA | 528 | UGCCUCCU UGAUG GCAUGCACUAUGC GCG GGCGUUCC | 1433 |
| 920 | GCAGCCUU G ACAUAAAU | 529 | AUUUAUGU UCAUG GCAUGCACUAUGC GCG AAGGCUGC | 1434 |
| 929 | ACAUAAAU G AUGGGCAU | 530 | AUGCCCAU UGAUG GCAUGCACUAUGC GCG AUUUAUGU | 1435 |
| 959 | UGGGCAGU G AAAUGAAU | 531 | AUUCAUUU UGAUG GCAUGCACUAUGC GCG ACUGCCCA | 1436 |
| 964 | AGUGAAAU G AAUGCGGC | 532 | GCCGCAUU UGAUG GCAUGCACUAUGC GCG AUUUCACU | 1437 |
| 968 | AAAUGAAU G CGGCCCUC | 533 | GAGGGCCG UCAUG GCAUGCACUAUGC GCG AUUCAUUU | 1438 |
| 979 | GCCCUCAU G CAUCGGAG | 534 | CUCCGAUG UGAUG GCAUGCACUAUGC GCG AUGAGGGC | 1439 |
| 1003 | GACCCAGU G CAGCUCCA | 535 | UGGAGCUG UGAUG GCAUGCACUAUGC GCG ACUGGGUC | 1440 |
| 1023 | GGCAGGGC G AGUGCGGU | 536 | ACCGCACU UCAUG GCAUGCACUAUGC GCG GCCCUGCC | 1441 |
| 1027 | GGGCGAGU G CGGUGGGC | 537 | GCCCACCG UGAUG GCAUGCACUAUGC GCG ACUCGCCC | 1442 |
| 1042 | GCCCGGGC G CUGUAUGA | 538 | UCAUACAG UGAUG GCAUGCACUAUGC GCG GCCCGGGC | 1443 |
| 1049 | CGCUGUAU G ACUUUGAG | 539 | CUCAAAGU UGAUG GCAUGCACUAUGC GCG AUACAGCG | 1444 |
| 1055 | AUGACUUU G AGGCCCUG | 540 | CAGGGCCU UGAUG GCAUGCACUAUGC GCG AAAGUCAU | 1445 |
| 1070 | UGGAGGAG G ACGAGCUG | 541 | CAGCUCGU UGAUG GCAUGCACUAUGC GCG AUCCUCCA | 1446 |
| 1073 | AGGAUGAC G AGCUGGGG | 542 | CCCCAGCU UGAUG GCAUGCACUAUGC GCG GUCAUCCU | 1447 |
| 1143 | GACCCGCC G CCUGCACA | 543 | UGUGCAGG UGAUG GCAUGCACUAUGC GCG GGCCGGUC | 1448 |
| 1147 | GGCCGCCU G CACAACAA | 544 | UUGUUGUG UCAUG GCAUGCACUAUGC GCG AGGCGGCC | 1449 |
| 1172 | UCUUCCCU G CCAACUAC | 545 | GUAGUUGG UGAUG GCAUGCACUAUGC GCG AGGGAAGA | 1450 |
| 1192 | CCACCCAU G ACCCGAUA | 546 | UAUCGGGU UGAUG GCAUGCACUAUGC GCG AUGGGUGC | 1451 |
| 1197 | CAUGACCC G AUAAACUC | 547 | GAGUUUAU UCAUG GCAUGCACUAUGC GCG GGGUCAUG | 1452 |
| 1237 | CUCGACCU G CCCACAAG | 548 | CUUGUGGG UGAUG GCAUGCACUAUGC GCG AGCUCCAG | 1453 |
| 1278 | GACUCCAU G ACUAUAUA | 549 | UAUAUAGU UGAUG GCAUGCACUAUGC GCG AUGGAGUC | 1454 |

Input Sequence = HSA011736.
Cut Site = YG/M or UG/U.
Stem Length = 8.
Core Sequence = UCAUG GCAUGCACUAUGC GCG
HSA011736 (Homo sapiens mRNA for growth factor receptor binding protein (GRBLG); 1303 bp)

TABLE VI

Human GRID Zinzyme and Substrate Sequence

| | | | | |
|---|---|---|---|---|
| Pos | Substrate | Seq ID | Zinzyme | Seq ID |

| | | | | |
|---|---|---|---|---|
| 11 | GAGGCACA G UUAAUGGA | 550 | UCCAUUAA GCCGAAAGGCGAGUCAAGGUCU UGUGCCUC | 1455 |
| 23 | AUGGAUCU G UAAACUUG | 551 | CAAGUUUA GCCGAAAGGCGAGUCAAGGUCU AGAUCCAU | 1456 |
| 31 | GUAAACUU G CACCCUCU | 493 | AGAGGGUG GCCGAAAGGCGAGUCAAGGUCU AAGUUUAC | 1457 |
| 46 | CUUUCAGA G UGGUACAU | 552 | AUGUACCA GCCGAAAGGCGAGUCAAGGUCU UCUGAAAG | 1458 |
| 49 | UCAGAGUG G UACAUGGA | 553 | UCCAUGUA GCCGAAAGGCGAGUCAAGGUCU CACUCUGA | 1459 |
| 63 | GGAAGACA G CACAAAGU | 554 | ACUUUGUG GCCGAAAGGCGAGUCAAGGUCU UGUCUUCC | 1460 |
| 70 | AGCACAAA G UGGAUCCA | 555 | UGGAUCCA GCCGAAAGGCGAGUCAAGGUCU UUUGUGCU | 1461 |
| 90 | UCUGAAAU G CAGUAACU | 495 | AGUUACUG GCCGAAAGGCGAGUCAAGGUCU AUGUCAGA | 1462 |
| 93 | GAAAUGCA G UAACUCUG | 556 | CAGAGUUA GCCGAAAGGCGAGUCAAGGUCU UGCAUUUC | 1463 |
| 104 | ACUCUGAU G CUUGAAUU | 497 | AAUUCAAG GCCGAAAGGCGAGUCAAGGUCU AUCAGAGU | 1464 |
| 114 | UUGAAUUU G UUCUCCCU | 557 | AGGGAGAA GCCGAAAGGCGAGUCAAGGUCU AAAUUCAA | 1465 |
| 127 | CCCUUCUU G CCAGAAAG | 499 | CUUUCUGG GCCGAAAGGCGAGUCAAGGUCU AAGAAGGG | 1466 |
| 151 | AUAACUCG G UGUCAAAG | 558 | CUCUGACA GCCGAAAGGCGAGUCAAGGUCU CGAGUUAU | 1467 |
| 153 | AACUCGGU G UCAAAGCC | 559 | GGCUUUGA GCCGAAAGGCGAGUCAAGGUCU ACCGAGUU | 1468 |
| 159 | GUGUCAAA G CCAAGACA | 560 | UGUCUUGG GCCGAAAGGCGAGUCAAGGUCU UUUGACAC | 1469 |
| 194 | UUCCAAAA G CUUCACGU | 561 | ACGUGAAG GCCGAAAGGCGAGUCAAGGUCU UUUUGGAA | 1470 |
| 201 | AGCUUCAC G UUACAGCA | 562 | UGCUGUAA GCCGAAAGGCGAGUCAAGGUCU GUGAAGCU | 1471 |
| 207 | ACGUUACA G CAUGGAAG | 563 | CUUCCAUG GCCGAAAGGCGAGUCAAGGUCU UGUAACGU | 1472 |
| 215 | GCAUGGAA G CUGUUGCC | 564 | GGCAACAG GCCGAAAGGCGAGUCAAGGUCU UUCCAUGC | 1473 |
| 218 | UGGAAGCU G UUGCCAAG | 565 | CUUGGCAA GCCGAAAGGCGAGUCAAGGUCU AGCUUCCA | 1474 |
| 221 | AAGCUGUU G CCAAGUUU | 500 | AAACUUGG GCCGAAAGGCGAGUCAAGGUCU AACAGCUU | 1475 |
| 226 | GUUGCCAA G UUUGAUUU | 566 | AAAUCAAA GCCGAAAGGCGAGUCAAGGUCU UUGGCAAC | 1476 |
| 239 | AUUUCACU G CUUCAGGU | 502 | ACCUGAAG GCCGAAAGGCGAGUCAAGGUCU AGUGAAAU | 1477 |
| 246 | UGCUUCAG G UGAGGAUG | 567 | CAUCCUCA GCCGAAAGGCGAGUCAAGGUCU CUGAAGCA | 1478 |
| 261 | UGAACUGA G CUCUCACA | 568 | UGUGAAAG GCCGAAAGGCGAGUCAAGGUCU UCAGUUCA | 1479 |
| 278 | CUGGAGAU G UUUUGAAG | 569 | CUUCAAAA GCCGAAAGGCGAGUCAAGGUCU AUCUCCAG | 1480 |
| 294 | GAUUUUAA G UAACCAAG | 570 | CUUGGUUA GCCGAAAGGCGAGUCAAGGUCU UUAAAAUC | 1481 |
| 307 | CAAGAGGA G UGGUUUAA | 571 | UUAAACCA GCCGAAAGGCGAGUCAAGGUCU UCCUCUUG | 1482 |
| 310 | GAGGAGUG G UUUAAGGC | 572 | GCCUUAAA GCCGAAAGGCGAGUCAAGGUCU CACUCCUC | 1483 |
| 317 | GGUUUAAG G CGGAGCUU | 573 | AAGCUCCG GCCGAAAGGCGAGUCAAGGUCU CUCCUUGC | 1494 |
| 322 | AAGGCGGA G CUUGGGAG | 574 | CUCCCAAG GCCGAAAGGCGAGUCAAGGUCU UCCGCCUU | 1485 |
| 330 | GCUUGGGA G CCAGGAAG | 575 | CUUCCUGG GCCGAAAGGCGAGUCAAGGUCU UCCCAAGC | 1486 |
| 344 | AAGGAUAU G UGCCCAAG | 576 | CUUGGGCA GCCGAAAGGCGAGUCAAGGUCU AUAUCCUU | 1487 |
| 346 | GGAUAUGU G CCCAAGAA | 507 | UUCUUGGG GCCGAAAGGCGAGUCAAGGUCU ACAUAUCC | 1488 |
| 370 | GACAUCCA G UUUCCCAA | 577 | UUGGGAAA GCCGAAAGGCGAGUCAAGGUCU UGGAUGUC | 1489 |
| 392 | CCCAAAUG G UCUCACGA | 578 | UCGUGAAA GCCGAAAGGCGAGUCAAGGUCU CAUUUGGG | 1490 |
| 393 | UCACGAAG G CCUCUCUC | 579 | GAGAGAGG GCCGAAAGGCGAGUCAAGGUCU CUUCGUGA | 1491 |
| 410 | GACACCAG G CAGAGAAC | 580 | GUUCUCUG GCCGAAAGGCGAGUCAAGGUCU CUGGUGUC | 1492 |
| 429 | ACUCAUGG G CAAGGAGG | 581 | CCUCCUUG GCCGAAAGGCGAGUCAAGGUCU CCAUGAGU | 1493 |
| 437 | GCAAGGAG G UUGGCUUC | 582 | GAAGCCAA GCCGAAAGGCGAGUCAAGGUCU CUCCUUGC | 1494 |
| 441 | GGAGGUUG G CUUCUUCA | 583 | UGAAGAAG GCCGAAAGGCGAGUCAAGGUCU CAACCUCC | 1495 |
| 458 | UCAUCCGG G CCAGCCAG | 584 | CUGGCUGG GCCGAAAGGCGAGUCAAGGUCU CCGGAUGA | 1496 |
| 462 | CCGGGCCA G CCAGAGCU | 585 | AGCUCUGG GCCGAAAGGCGAGUCAAGGUCU UGGCCCGG | 1497 |
| 468 | CAGCCAGA G CUCCCCAG | 586 | CUGGGGAG GCCGAAAGGCGAGUCAAGGUCU UCUGGCUG | 1498 |
| 494 | CCAUCUCU G UCAGGCAU | 587 | AUGCCUGA GCCGAAAGGCGAGUCAAGGUCU AGAGAUGG | 1499 |
| 499 | UCUGUCAG G CAUGAGGA | 588 | UCCUCAUG GCCGAAAGGCGAGUCAAGGUCU CUGACAGA | 1500 |
| 512 | AGGAUGAC G UUCAACAC | 589 | GUGUUGAA GCCGAAAGGCGAGUCAAGGUCU GUCAUCCU | 1501 |
| 527 | ACUUCAAG G UCAUGCGA | 590 | UCGCAUGA GCCGAAAGGCGAGUCAAGGUCU CUUGAAGU | 1502 |
| 532 | AAGGUCAU G CGAGACAA | 512 | UUGUCUCG GCCGAAAGGCGAGUCAAGGUCU AUGACCUU | 1503 |
| 546 | CAACAAGG G UAAUUACU | 591 | AGUAAUUA GCCGAAAGGCGAGUCAAGGUCU CCUUGUUG | 1504 |
| 559 | UACUUUCU G UGGACUGA | 592 | UCAGUCCA GCCGAAAGGCGAGUCAAGGUCU AGAAAGUA | 1505 |
| 571 | ACUGAGAA G UUUCCAUC | 593 | GAUGGAAA GCCGAAAGGCGAGUCAAGGUCU UUCUCAGU | 1506 |
| 589 | CUAAAUAA G CUGGUAGA | 594 | UCUACCAG GCCGAAAGGCGAGUCAAGGUCU UUAUUUAG | 1507 |
| 593 | AUAAGCUG G UAGACUAC | 595 | GUAGUCUA GCCGAAAGGCGAGUCAAGGUCU CAGCUUAU | 1508 |
| 631 | AGACAGAA G CAGAUCUU | 596 | AAGAUCUG GCCGAAAGGCGAGUCAAGGUCU UUCUGUCU | 1509 |
| 669 | AGACCAGG G UCACCGGG | 597 | CCCGGUGA GCCGAAAGGCGAGUCAAGGUCU CCUGGUCU | 1510 |
| 678 | UCACCGGG G CAACAGCC | 598 | GGCUGUUG GCCGAAAGGCGAGUCAAGGUCU CCCGGUGA | 1511 |
| 684 | GGGCAACA G CCUGGACC | 599 | GGUCCAGG GCCGAAAGGCGAGUCAAGGUCU UGUUGCCC | 1512 |
| 697 | GACCGGAG G UCCCAGGG | 600 | CCCUGGGA GCCGAAAGGCGAGUCAAGGUCU CUCCGGUC | 1513 |
| 708 | CCAGGGAG G CCCACACC | 601 | GGUGUGGG GCCGAAAGGCGAGUCAAGGUCU CUCCCUGG | 1514 |
| 720 | ACACCUCA G UGGGGCUG | 602 | CAGCCCCA GCCGAAAGGCGAGUCAAGGUCU UGAGGUGU | 1515 |
| 725 | UCAGUGGG G CUGUGGGA | 603 | UCCCACAG GCCGAAAGGCGAGUCAAGGUCU CCCACUGA | 1516 |
| 728 | GUGGGGCU G UGGGAGAA | 604 | UUCUCCCA GCCGAAAGGCGAGUCAAGGUCU AGCCCCAC | 1517 |
| 763 | AACCGGAA G CUGUCGGA | 605 | UCCGACAG GCCGAAAGGCGAGUCAAGGUCU UUCCGGUU | 1518 |
| 766 | CGGAAGCU G UCGGAUCA | 606 | UGAUCCGA GCCGAAAGGCGAGUCAAGGUCU AGCUUCCG | 1519 |
| 793 | CUUCCCCU G CAGCAGCA | 520 | UGCUGCUG GCCGAAAGGCGAGUCAAGGUCU AGGGGAAG | 1520 |
| 796 | CCCCUGCA G CAGCACCA | 607 | UGGUGCUG GCCGAAAGGCGAGUCAAGGUCU UGCAGGGG | 1521 |
| 799 | CUGCAGCA G CACCAGCA | 608 | UGCUGGUG GCCGAAAGGCGAGUCAAGGUCU UGCUGCAG | 1522 |
| 805 | CAGCACCA G CACCAGCC | 609 | GGCUGGUG GCCGAAAGGCGAGUCAAGGUCU UGGUGCUG | 1523 |
| 811 | CAGCACCA G CCACAGCC | 610 | GGCUGUGG GCCGAAAGGCGAGUCAAGGUCU UGGUGCUG | 1524 |
| 817 | CAGCCACA G CCUCCGCA | 611 | UGCGGAGG GCCGAAAGGCGAGUCAAGGUCU UGUGGCUG | 1525 |
| 823 | CAGCCUCC G CAAUAUGC | 521 | GCAUAUUG GCCGAAAGGCGAGUCAAGGUCU GGAGGCUG | 1526 |
| 830 | CGCAAUAU G CCCCAGCG | 522 | CGCUGGGG GCCGAAAGGCGAGUCAAGGUCU AUAUUGCG | 1527 |
| 836 | AUGCCCCA G CGCCCCAG | 612 | CUGGGGCG GCCGAAAGGCGAGUCAAGGUCU UGGGGCAU | 1528 |
| 838 | GCCCCAGC G CCCCAGCA | 523 | UGCUGGGG GCCGAAAGGCGAGUCAAGGUCU GCUGGGGC | 1529 |
| 844 | GCGCCCCA G CAGCUGCA | 613 | UGCAGCUG GCCGAAAGGCGAGUCAAGGUCU UGGGGCGC | 1530 |
| 847 | CCCCAGCA G CUGCAGCA | 614 | UGCUGCAG GCCGAAAGGCGAGUCAAGGUCU UGCUGGGG | 1531 |
| 850 | CAGCAGCU G CAGCAGCC | 524 | GGCUGCUG GCCGAAAGGCGAGUCAAGGUCU AUCUGCUG | 1532 |
| 853 | CAGCUGCA G CAUCCCCC | 615 | GGGGGCUG GCCGAAAGGCGAGUCAAGGUCU UGCAGCUG | 1533 |
| 856 | CUGCAGCA G CCCCCACA | 616 | UGUGGGGG GCCGAAAGGCGAGUCAAGGUCU UGCUGCAG | 1534 |
| 865 | CCCCCACA G CAGCGAUA | 617 | UAUCGCUG GCCGAAAGGCGAGUCAAGGUCU UGUGGGGG | 1535 |
| 868 | CCACAGCA G CGAUAUCU | 618 | AGAUAUCG GCCGAAAGGCGAGUCAAGGUCU UGCUGUGG | 1536 |
| 877 | CGAUAUCU G CAGCACCA | 526 | UGGUGCUG GCCGAAAGGCGAGUCAAGGUCU AGAUAUCG | 1537 |
| 880 | UAUCUGCA G CACCACCA | 619 | UGGUGGUG GCCGAAAGGCGAGUCAAGGUCU UGCAGAUA | 1538 |
| 903 | CCAGGAAC G CCGAGGAG | 527 | CUCCUCGG GCCGAAAGGCGAGUCAAGGUCU GUUCCUGG | 1539 |
| 912 | CCGAGGAG G CAGCCUUG | 620 | CAAGGCUG GCCGAAAGGCGAGUCAAGGUCU CUCCUCGG | 1540 |
| 915 | AGGAGGCA G CCUUGACA | 621 | UGUCAAGG GCCGAAAGGCGAGUCAAGGUCU UGCCUCCU | 1541 |
| 934 | AAUGAUGG G CAUUGUGG | 622 | CCACAAUG GCCGAAAGGCGAGUCAAGGUCU CCAUCAUU | 1542 |
| 939 | UGGGCAUU G UGGCACCG | 623 | CGGUGCCA GCCGAAAGGCGAGUCAAGGUCU AAUGCCCA | 1543 |
| 942 | GCAUUGUG G CACCGGCU | 624 | AGCCGGUG GCCGAAAGGCGAGUCAAGGUCU CACAAUGC | 1544 |
| 948 | UGGCACCG G CUUGGGCA | 625 | UGCCCAAG GCCGAAAGGCGAGUCAAGGUCU CGGUGCCA | 1545 |
| 954 | CGGCUUGG G CAGUGAAA | 626 | UUUCACUG GCCGAAAGGCGAGUCAAGGUCU CCAAGCCG | 1546 |
| 957 | CUCGGGCA G UGAAAUGA | 627 | UCAUUUCA GCCGAAAGGCGAGUCAAGGUCU UGCCCAAG | 1547 |
| 968 | AAAUGAAU G CGGCCCUC | 533 | GAGGGCCG GCCGAAAGGCGAGUCAAGGUCU AUUCAUUU | 1548 |
| 971 | UGAAUGCG G CCCUCAUG | 628 | CAUGAGGG GCCGAAAGGCGAGUCAAGGUCU CGCAUUCA | 1549 |
| 979 | GCCCUCAU G CAUCGGAG | 534 | CUCCGAUG GCCGAAAGGCGAGUCAAGGUCU AUGAGGGC | 1550 |
| 1001 | CAGACCCA G UGCAGCUC | 629 | GAGCUGCA GCCGAAAGGCGAGUCAAGGUCU UGGGUCUG | 1551 |
| 1003 | GACCCAGU G CAGCUCCA | 535 | UGGAGCUG GCCGAAAGGCGAGUCAAGGUCU ACUGGGUC | 1552 |
| 1006 | CCAGUGCA G CUCCAGGC | 630 | GCCUGGAG GCCGAAAGGCGAGUCAAGGUCU UGCACUGG | 1553 |
| 1013 | AGCUCCAG G CGGCAGGG | 631 | CCCUGCCG GCCGAAAGGCGAGUCAAGGUCU CUGGAGCU | 1554 |
| 1016 | UCCAGGGG G CAGGGCGA | 632 | UCGUCCUG GCCGAAAGGCGAGUCAAGGUCU CGCCUGGA | 1555 |
| 1021 | GCGGCAGG G CGAGUGCG | 633 | CGCACUCG GCCGAAAGGCGAGUCAAGGUCU CCUGCCGC | 1556 |
| 1025 | CAGGGCGA G UGCGGUGG | 634 | CCACCGCA GCCGAAAGGCGAGUCAAGGUCU UCGCCCUG | 1557 |
| 1027 | GGGCGACU G CGGUGGGC | 537 | GCCCACCG GCCGAAAGGCGAGUCAAGGUCU ACUCGCCC | 1558 |
| 1030 | CGAGUGCG G UGGGCCCG | 635 | CGGGCCCA GCCGAAAGGCGAGUCAAGGUCU CGCACUCG | 1559 |
| 1034 | UGCGGUGG G CCCGGGCG | 636 | CGCCCGGG GCCGAAAGGCGAGUCAAGGUCU CCACCGCA | 1560 |
| 1040 | GGGCCCGG G CGCUGUAU | 637 | AUACAGCG GCCGAAAGGCGAGUCAAGGUCU CCGGGCCC | 1561 |
| 1042 | GCCCGGGC G CUGUACGA | 538 | UCAGACAG GCCGAAAGGCGAGUCAAGGUCU GCCCGGGC | 1562 |
| 1045 | CGGGCGCU G UAUGACUU | 638 | AAGUCAUA GCCGAAAGGCGAGUCAAGGUCU AGCGCCCG | 1563 |
| 1058 | ACUCUGAG G CCCUGGAG | 639 | CUCCAGGG GCCGAAAGGCGAGUCAAGGUCU CUCAAAGU | 1564 |
| 1075 | GAUGACGA G CUGGGGUU | 640 | AACCCCAG GCCGAAAGGCGAGUCAAGGUCU UCGUCAUC | 1565 |
| 1081 | GAGCUGGG G UUCCACAG | 641 | CUGUGGAA GCCGAAAGGCGAGUCAAGGUCU CCCAGCUC | 1566 |
| 1089 | GUUCCACA G CGGGGACC | 642 | CCUCCCCG GCCGAAAGGCGAGUCAAGGUCU UGUGGAAC | 1567 |
| 1097 | GCGGGGAG G UGGUGGAG | 643 | CUCCACCA GCCGAAAGGCGAGUCAAGGUCU CUCCCCGC | 1568 |
| 1100 | GGGAGGUG G UGGAGGUC | 644 | GACCUCCA GCCGAAAGGCGAGUCAAGGUCU CACCUCCC | 1569 |
| 1106 | UGGUGGAG G UCCUGGAU | 645 | AUCCAGGA GCCGAAAGGCGAGUCAAGGUCU CUCCACCA | 1570 |
| 1116 | CCUGGAUA G CUCCAACC | 646 | GGUUGGAG GCCGAAAGGCGAGUCAAGGUCU UAUCCAGG | 1571 |
| 1132 | CCAUCCUG G UGGACCGG | 647 | CCGGUCCA GCCGAAAGGCGAGUCAAGGUCU CAGGAUGG | 1572 |
| 1140 | GUGGACCG G CCGCCUGC | 648 | GCAGGCGG GCCGAAAGGCGAGUCAAGGUCU CGGUCCAC | 1573 |
| 1143 | GACCGGCC G CCUGCACA | 543 | UGUGCAGG GCCGAAAGGCGAGUCAAGGUCU GGCCGGUC | 1574 |
| 1147 | GGCCGCCU G CACAACAA | 544 | UUGUUGUG GCCGAAAGGCGAGUCAAGGUCU AGGCGGCC | 1575 |
| 1156 | CACAACAA G CUGGGCCU | 649 | AGGCCCAG GCCGAAAGGCGAGUCAAGGUCU UUGUUGUG | 1576 |
| 1161 | CAAGCUGG G CCUCUUCC | 650 | GGAAGAGG GCCGAAAGGCGAGUCAAGGUCU CCAGCUUG | 1577 |
| 1172 | UCUUCCCU G CCAACUAC | 545 | GUAGUUGG GCCGAAAGGCGAGUCAAGGUCU AGGGAAGA | 1578 |
| 1181 | CCAACUAC G UGGCACCC | 651 | GGGUGCCA GCCGAAAGGCGAGUCAAGGUCU GUAGUUGG | 1579 |
| 1184 | ACUACGUG G CACCCAUG | 652 | CAUGGGUG GCCGAAAGGCGAGUCAAGGUCU CACGUAGU | 1580 |
| 1220 | GGACAGAA G CUUUUUGU | 653 | ACAAAAAG GCCGAAAGGCGAGUCAAGGUCU UUCUGUCC | 1581 |
| 1227 | AGCUUUUU G UCUGGAGC | 654 | GCUCCAGA GCCGAAAGGCGAGUCAAGGUCU AAAAAGCU | 1582 |
| 1234 | UGUCUGGA G CUGCCCAC | 655 | GUGGGCAG GCCGAAAGGCGAGUCAAGGUCU UCCAGACA | 1583 |
| 1237 | CUGGAGCU G CCCACAAG | 548 | CUUGUGGG GCCGAAAGGCGAGUCAAGGUCU AGCUCCAG | 1584 |
| 1253 | GAAAGAGG G CAAGGAAA | 656 | UUUCCUUG GCCGAAAGGCGAGUCAAGGUCU CCUCUUUC | 1585 |
| 1266 | GAAAAAAG G CUGGACUC | 657 | GAGUCCAG GCCGAAAGGCGAGUCAAGGUCU CUUUUUUC | 1586 |

Input Sequence = HSA011736.
Cut Site = G/Y
Stem Length = 8.
Core Sequence = GCcgaaagGCGaGuCaaGGuCu
HSA011736 (Homo sapiens mRNA for growth factor receptor binding protein (GRBLG); 1303 bp)

TABLE VII

Human GRID DNAzyme and Substrate Sequence

| | | | | |
|---|---|---|---|---|
| | | Seq | | Seq |
| Pos | Substrate | ID | DNAzyme | ID |

| | | | | |
|---|---|---|---|---|
| 11 | GAGGCACA G UUAAUGGA | 550 | TCCATTAA GGCTAGCTACAACGA TGTGCCTC | 1587 |
| 15 | CACAGUUA A UGGAUCUG | 658 | CAGATCCA GGCTAGCTACAACGA TAACTGTG | 1588 |
| 19 | GUUAAUGG A UCUGUAAA | 659 | TTTACAGA GGCTAGCTACAACGA CCATTAC | 1589 |
| 23 | AUGGAUCU G UAAACUUG | 551 | CAAGTTTA GGCTAGCTACAACGA AGATCCAT | 1590 |
| 27 | AUCUGUAA A CUUGCACC | 660 | GGTGCAAG GGCTAGCTACAACGA TTACAGAT | 1591 |
| 31 | GUAAACUU G CACCCUCU | 493 | AGAGGUTO GGCTAGCTACAACGA AAGTTTAC | 1592 |
| 33 | AAACUUGC A CCCUCUUU | 183 | AAAGAGGG GGCTAGCTACAACGA GCAAGTTT | 1593 |
| 46 | CUUUCAGA G UGGUACAU | 552 | ATGTAOCA GGCTAGCTACAACGA TCTGAAAG | 1594 |
| 49 | UCAGAGUG G UACAUGGA | 553 | TCCATGTA GGCTAGCTACAACGA CACTCTGA | 1595 |
| 51 | AGAGUGGU A CAUGGAAG | 10 | CTTCCATG GGCTAGCTACAACGA ACCACTOT | 1596 |
| 53 | AGUGGUAC A UGGAAGAC | 189 | GTCTTCCA GGCTAGCTACAACGA UTACCACT | 1597 |
| 60 | CAUGGAAG A CAGCACAA | 661 | TTGTGCTG GGCTAGCTACAACGA CTTCCATG | 1599 |
| 63 | GGAAGACA G CACAAAGU | 554 | ACTTTGTG GGCTAGCTACAACGA TGTCTTCC | 1599 |
| 65 | AAGACAGC A CAAAGUGG | 191 | CCACTTTG GGCTAGCTACAACGA GCTGTCTT | 1600 |
| 70 | AGCACAAA G UGGAUCCA | 555 | TGGATCCA GGCTAGCTACAACGA TTTGTGCT | 1601 |
| 74 | CAAAGUGG A UCCAUACU | 662 | AUTATOGA GGCTAGCTACAACGA CCACTTTG | 1602 |
| 78 | GUGGAUCC A UACUCUGA | 194 | TCAGAGTA GGCTAGCTACAACGA GGATCCAC | 1603 |
| 80 | GGAUCCAU A CUCUGAAA | 12 | TTTCAGAG GGCTAGCTACAACGA ATGGATCC | 1604 |
| 88 | ACUCUGAA A UGCAGUAA | 663 | TTACTGCA GGCTAGCTACAACGA TTCAGAGT | 1605 |
| 90 | UCUGAAAU G CAGUAACU | 495 | AGTTACTG GGCTAGCTACAACGA ATTTCAGA | 1606 |
| 93 | GAAAUGCA G UAACUCUG | 556 | CAGAGTTA GGCTAGCTACAACGA TGCATTTC | 1607 |
| 96 | AUGCAGUA A CUCUGAUG | 664 | CATOAGAG GGCTAGCTACAACGA TACTUCAT | 1608 |
| 102 | UAACUCUG A UGCUUGAA | 665 | TTCAAGCA GGCTAGCTACAACGA CAGAGTTA | 1609 |
| 104 | ACUCUGAU G CUUGAAUU | 497 | AATTCAAG GUCTAGCTACAACGA ATCAGAUT | 1610 |
| 110 | AUGCUUGA A UUUGUUCU | 666 | AGAACAAA GGCTAGCTACAACGA TCAAGCAT | 1611 |
| 114 | UUGAAUUU G UUCUCCCU | 557 | AGGGAUAA GGCTAGCTACAACGA AAATTCAA | 1612 |
| 127 | CCCUUCUU G CCAGAAAG | 499 | CTTTCTGG GGCTAGCTACAACGA AAGAAGGU | 1613 |
| 137 | CAGAAAGG A UUCUAAUA | 667 | TATTAGAA GGCTAGCTACAACGA CCTTTCTG | 1614 |
| 143 | GGAUUCUA A UAACUCGG | 668 | CCGAGTTA GGCTAGCTACAACGA TAGAATCC | 1615 |
| 146 | UUCUAACA A CUCGGUGC | 669 | ACACCUAG GGCTAGCTACAACGA TATTAGAA | 1616 |
| 151 | AUAACUCG G UGUCAAAG | 558 | CTTTGACA GGCTAUCTACAACGA CGAUTTAT | 1617 |
| 153 | AACUCGGU G UCAAAGCC | 559 | UGCTTTGA GGCTAGCTACAACGA ACCUAGTT | 1618 |
| 159 | GUGUCAAA G CCAAGACA | 560 | TUTOTTUG GGCTAGCTACAACGA TTTUACAC | 1619 |
| 165 | AAGCCAAG A CAUAAACU | 670 | AUTTTATG UGCTAGCTACAACGA CTTGGCTT | 1620 |
| 167 | GCCAAGAC A UAAACUCA | 213 | TGAGTTTA GGCTAGCTACAACGA GTCTTGUC | 1621 |
| 171 | AGACAUAA A CUCAAUCU | 671 | AGATTUAG GGCTAGCTACAACGA TTATUTCT | 1622 |
| 176 | UAAACUCA A UCUCUUCU | 672 | AGAAGAGA GGCTAGCTACAACGA TGAGTTTA | 1623 |
| 194 | UUCCAAAA G CUUCACGU | 561 | ACGTGAAG GGCTAGCTACAACGA TTTTGGAA | 1624 |
| 199 | AAAGCUUC A CGUUACAG | 223 | CTGTAACG GGCTAGCTACAACGA GAAGCTTT | 1625 |
| 201 | AGCUUCAC G UUACAGCA | 562 | TGCTGTAA GGCTAGCTACAACGA GTGAAGCT | 1626 |
| 204 | UUCACGUU A CAGCAUGG | 43 | CCATGCTG GUCTAGCTACAACGA AACGTGAA | 1627 |
| 207 | ACGUUACA G CAUGGAAG | 563 | CTTCCATU GGCTAGCTACAACGA TGTAACGT | 1628 |
| 209 | GUUACAGC A UGGAAGCU | 225 | AGCTTCCA GGCTAGCTACAACGA GCTGTAAC | 1629 |
| 215 | GCAUGGAA G CUGUUGCC | 564 | GGCAACAG GGCTAGCTACAACGA TTCCATUC | 1630 |
| 218 | UGGAAGCU G UUGCCAAG | 565 | CTTGGCAA GUCTAUCTACAACGA AUCTTCCA | 1631 |
| 221 | AAGCUGUU G CCAAGUUU | 500 | AAACTTGG GGCTAGCTACAACGA AACAUCTT | 1632 |
| 226 | GUUGCCAA G UUUGAUUU | 566 | AAATCAAA GGCTAGCTACAACGA TTGGCAAC | 1633 |
| 231 | CAAGUUUG A UUUCACUG | 673 | CAGTGAAA GGCTAGCTACAACGA CAAACTTG | 1634 |
| 236 | UUGAUUUC A CUGCUUCA | 229 | TGAAGCAC GGCTAGCTACAACGA GAAATCAA | 1635 |
| 239 | AUUUCACU G CUUCAGGU | 502 | ACCTGAAC GCCTAGCTACAACGA ACTGAAAT | 1636 |
| 246 | UGCUUCAG G UGAGGAUG | 567 | CATCCTCA GGCTAGCTACAACGA CTGAAGCA | 1637 |
| 252 | AGGUGAGG A UGAACUGA | 674 | TCAGTTCA GGCTAGCTACAACGA CCTCACCT | 1638 |
| 256 | GAGGAUGA A CUGAGCUU | 675 | AAGCTCAG GGCTAGCTACAACGA TCATCCTC | 1639 |
| 261 | UGAACUGA G CUUUCACA | 568 | TGTGAAAG GGCTAGCTACAACGA TCAGTTCA | 1640 |
| 267 | GAGCUUUC A CACUGGAG | 235 | CTCCAGTG GGCTAGCTACAACGA GAAAGCTC | 1641 |
| 269 | GCUUUCAC A CUGGAGAU | 236 | ATCTCCAG GGCTAGCTACAACGA GTGAAAGC | 1642 |
| 276 | CACUGGAG A UGUUUUGA | 676 | TCAAAACA GGCTAGCTACAACGA CTCCAGTG | 1643 |
| 278 | CUGGAGAU G UUUUGAAG | 569 | CTTCAAAA GGCTAGCTACAACGA ATUTOCAG | 1644 |
| 287 | UUUUGAAG A UUUUAAGU | 677 | ACTTAAAA GGCTAGCTACAACGA CTTCAAAA | 1645 |
| 294 | GAUUUUAA G UAACCAAG | 570 | CTTGGTTA GGCTAGCTACAACGA TTAAAATC | 1646 |
| 297 | UUUAAGUA A CCAAGAGG | 678 | CCTCTTGG GGCTAGCTACAACGA TACTTAAA | 1647 |
| 307 | CAAGAGGA G UGGUUUAA | 571 | TTAAACCA GGCTAGCTACAACGA TCCTCTTG | 1648 |
| 310 | GAGGAGUG G UUUAAGGC | 572 | GCCTTAAA GGCTAGCTACAACGA CACTCCTC | 1649 |
| 317 | GGUUUAAG C CGGAGCUU | 573 | AAGCTCCG GGCTAGCTACAACGA CTTAAACC | 1650 |
| 322 | AAGGCGGA G CUUGGGAG | 574 | CTCCCAAG GGCTAGCTACAACGA TCCGCCTT | 1651 |
| 330 | GUUUGGGA U CCAGGAAG | 575 | CTTCCTGG GGCTAGCTACAACGA TCCCAAGC | 1652 |
| 340 | CAGGAAGG A UAUGUGCC | 679 | GGCACATA GGCTAGCTACAAUGA CCTTCCTG | 1653 |
| 342 | GGAAGGAU A UGUGCCCA | 67 | TGGGCACA GGCTAGCTACAACGA ATCCTTCC | 1654 |
| 344 | AAGGAUAU G UGCCCAAG | 576 | CTTGGGCA GGCTAGCTACAACGA ATATCCTT | 1655 |
| 346 | GGAUAUGU G CCCAAGAA | 507 | TTCTTGGG GGCTAGCTACAACGA ACATATCC | 1656 |
| 354 | GCCCAAGA A UUUCAUAG | 680 | CTATGAAA GGCTAGCTACAACGA TCTTGGGC | 1657 |
| 359 | AGAAUUUC A UAGACAUC | 246 | GATGTCTA GGCTAGCTACAACGA GAAATTCT | 1658 |
| 363 | UCUCAGAG A CAUCCAGU | 681 | ACTGGATG GGCTAGCTACAACGA CTATGAAA | 1659 |
| 365 | UCACAGAC A UCCAGUUU | 247 | AAACTGGA GGCTAGCTACAACGA GTCTATGA | 1660 |
| 370 | GACAUCCA C UUUCCCAA | 577 | TTGGGAAA GGCTAGCTACAACGA TGGATGTC | 1661 |
| 379 | UUUCCCAA A UGGUCUCA | 682 | TGAAACCA GGCTAGCTACAACGA TTGGGAAA | 1662 |
| 382 | CCCAAAUG U UUUCACGA | 578 | TCGTGAAA GGCTAGCTACAACGA CATTTGGG | 1663 |
| 387 | AUGGUCUC A CGAAGGCU | 253 | GGCCTTCG GGCTAGCTACAACGA GAAACCAT | 1664 |
| 393 | UCACGAAG C CCUCUCUC | 579 | GAGAGAGG GGCTAGCTACAACGA CTTCGTGA | 1665 |
| 403 | CUCUCUCO A CACCAGGC | 683 | GCCTGGTG GGCTAGCTACAAUGA CGAGAGAG | 1666 |
| 405 | CUCUCGAC A CCAGGCAG | 258 | CTGCCTGG GGCTAGCTACAACGA GTCGAGAG | 1667 |
| 410 | GACACCAG C CAGAGAAC | 580 | GTTCTCTG GGCTAGCTACAACGA CTGGTGTC | 1668 |
| 417 | GGCAGAGA A CUUACUCA | 684 | CGAGTAAG GGCTAGCTACAACGA TCTCTGCC | 1669 |
| 421 | GAGAACUU A CUCAUGGG | 83 | CCCATGAG GGCTAGCTACAACGA AAGTTCTC | 1670 |
| 425 | ACUWACUC A UGGGCAAG | 264 | CTTGCCCA GGCTAGCTACAACGA GAGTAAGT | 1671 |
| 429 | ACUCAUGO U CAAGGAGG | 581 | CCTCCTTG GGCTAGCTACAACGA CCATGAGT | 1672 |
| 437 | GCAAGGAG G UUGGCUUC | 582 | GAAGCCAA GGCTAGCTACAACGA CCATGAGT | 1673 |
| 441 | GGAGGUUG C CUUCUUCA | 583 | TGAAGAAG GGCTAGCTACAACCA CAACCTCC | 1674 |
| 449 | GCUUCUUC A UCAUCCUG | 268 | CCGGATGA GGCTAGCTACAACGA GAAGAAGC | 1675 |
| 452 | UCUOCAUC A UCCGGGCC | 269 | GGCCCGGA GGCTAGCTACAACGA GATGAAGA | 1676 |
| 458 | UCAUCCUG U CCAGCCAG | 584 | CTGGCTGG GGCTAGCTACAACGA CCGGATGA | 1677 |
| 462 | UCOGUCCA C CCAGAGCU | 585 | AGCTCTCG GCCTACCTACAACCA TGGCCCGG | 1678 |
| 468 | CAUCCAGA C CUCCOCAG | 586 | CTGGGGAG GGCTAGCTACAACGA TCTGCCTG | 1679 |
| 480 | UCCAGGUG A CUUCUCCA | 685 | TGGAGAAG CGCTAGCTACAACGA CCCCTGGG | 1680 |
| 488 | ACUUCUCC A UCUCUGUC | 283 | CACAGAGA GGCTAGCTACAACGA GGACAAGT | 1681 |
| 494 | CCAUCUCU G UCAGGCAU | 587 | ATGCCTGA GGCTAGCTACAACGA AGAGATGG | 1682 |
| 499 | UCUGUCAG C CAUCACCA | 588 | TCCTCATG CGCTAGCTACAACGA CTCACACA | 1682 |
| 501 | UGUCAGUC A UGAGGAUG | 287 | CATCCTCA GGCLAGCTACAACGA GCCTGACA | 1684 |
| 507 | GCAUGACG A UGACGUUC | 686 | GAACGTCA GGCTAGCTACAACGA CCTCATGC | 1685 |
| 510 | UGAGGAUG A CGUUCAAC | 687 | GTTGAACG GGCTAGCTACAACGA CATCCTCA | 1686 |
| 512 | AGGAUGAC C UUCAACAC | 589 | GTGTTGAA GGCTAGCTACAACGA GTCATCCT | 1687 |
| 517 | GACUCUCA A CACUUCAA | 688 | TTGAAGTG GGCTAGCTACAACGA TGAACGTC | 1688 |
| 519 | CGUUCAAC A CUUCAAGG | 289 | CCTTGAAG GGCTAGCTACAACGA GTTGAACG | 1689 |
| 527 | ACUUCAAG C UCAUGCGA | 590 | TCGCATGA GGCTAGCTACAACGA CTTGAAGT | 1690 |
| 530 | UCAAGGUC A UGGUAGAC | 292 | GTCTCGCA GGCTAGCTACAACGA GACCTTGA | 1691 |
| 532 | AAGGUCAU G CGAGACAA | 512 | TTGTCTCG GGCTAGCTACAACGA ATGACCTT | 1692 |
| 537 | CAUGCGAG A CAACAAGG | 689 | CCTTGTTG GGCTAGCTACAACGA CTCGCATG | 1693 |
| 540 | GCGAGACA A CAAGGGUA | 690 | TACCCTTG GGCTAGCTACAACGA TGTCTCGC | 1694 |
| 546 | CAACAAGG C UAAUUACU | 591 | ACTAATTA GGCTAGCTACAACGA CCTTGTTG | 1695 |
| 549 | CAAGGGUA A UUACUUUC | 691 | GAAAGTAA GGCTAGCTACAACGA TACCCTTG | 1696 |
| 552 | GGGUAAUU A CUUUCUGU | 106 | ACAGAAAG GGCTAGCTACAACGA AATTACCC | 1697 |
| 559 | UACGUUCU G UGGACUGA | 592 | TCAGTCCA GGCTAGCTACAACGA AGAAAGTA | 1698 |
| 563 | UUCUGUGG A CUGAGAAG | 692 | CTTCTCAG GGCTAGCTACAACGA CCACAGAA | 1699 |
| 571 | ACUGAGAA G UUUCCAUC | 593 | GATGGAAA GGCTAGCTACAACGA TTCTCAGT | 1700 |
| 577 | AAGUUUCC A UCCCUAAA | 299 | TTTAGGGA GGCTAGCTACAACGA GGAAACTT | 1701 |
| 585 | AUCCCUAA A UAAGCUGG | 693 | CCAGCTTA GGCTAGCTACAACGA TTAGGGAT | 1702 |
| 589 | CUAAAUAA G CUGGUAGA | 594 | TCTACCAG GGCTAGCTACAACGA TTATTTAG | 1703 |
| 593 | AUAAGCUG G UAGACUAC | 595 | GTAGTCTA GGCTAGCTACAACGA CAGCTTAT | 1704 |
| 597 | UCUGGUAG A CUACUACA | 694 | TGTAGTAG GGCTAGCTACAACGA CTACCAGC | 1705 |
| 600 | GGUAGACU A CUACAGGA | 117 | TCCTGTAG GGCTAGCTACAACGA AGTCTACC | 1706 |
| 603 | AGACUACU A CAGGACAA | 118 | TTGTCCTG GGCTAGCTACAACGA AGTAGTCT | 1707 |
| 608 | ACUACAGG A CAAAUUCC | 695 | GGAATTTG GGCTAGCTACAACGA CCTGTAGT | 1708 |
| 612 | CAGGACAA A UUCCAUCU | 696 | AGATGGAA GGCTAGCTACAACGA TTGTCCTG | 1709 |
| 617 | CAAAUUCC A UCUCCAGA | 309 | TCTGGAGA GGCTAGCTACAACGA GGAATTTG | 1710 |
| 625 | AUCUCCAG A CAGAAGCA | 697 | TGCTTCTG GGCTAGCTACAACGA CTGGAGAT | 1711 |
| 631 | AGACAGAA G CAGAUCUU | 596 | AAGATCTG GGCTAGCTACAACGA TTCTGTCP | 1712 |
| 635 | AGAAGCAG A UCUUCCUU | 698 | AAGGAAGA GGCTAGCTACAACGA CTGCTTCT | 1713 |
| 648 | CCUUAGAG A CAGAACCC | 699 | GGGTTCTG GGCTAGCTACAACGA CTCTAAGG | 1714 |
| 653 | GAGACAGA A CCCGAGAA | 700 | TTCTCGGG GGCTAGCTACAACGA TCTGTCTC | 1715 |
| 663 | CCGAGAAG A CCAGGGUC | 701 | GACCCTGG GGCTAGCTACAACGA CTTCTCGG | 1716 |
| 669 | AGACCAGG C UCACCGGG | 597 | CCCGGTGA GGCTAGCTACAACGA CCTGGTCT | 1717 |
| 672 | CCAGGGUC A CCGGGGCA | 323 | TGCCCCGG GGCTAGCTACAACGA GACCCTGG | 1718 |
| 678 | UCACCGGG C CAACAGCC | 598 | GGCTGTTG GCCTAGCTACAACCA CCCGGTGA | 1719 |
| 681 | CCGGGGCA A CAGCCUGG | 702 | CCAGGCTG GGCTAGCTACAACGA TGCCCCGG | 1720 |
| 684 | GGGCAACA C CCUCGACC | 599 | GGTCCAGG GGCTAGCTACAACGA TGTTGCCC | 1721 |
| 690 | CAGCCUGG A CCGGACGU | 703 | ACCTCCGG GGCTAGCTACAACGA CCAGGCTG | 1722 |
| 697 | GACCGGAG C UCCCAGGG | 600 | CCCTGGGA GGCTAGCTACAACGA CTCCGCTC | 1723 |
| 708 | CCAGGGAG C CCCACACC | 601 | GGTGTGCG GGCTAGCTACAACGA CTCCCTGG | 1724 |
| 712 | GGAGGCCC A CACCUCAG | 335 | CTGAGGTC GGCTAGCTACAACGA GGCCCTCC | 1725 |
| 714 | AGGCCCAC A CCUCAGUG | 336 | CACTGAGG GGCTAGCTACAACGA GTCGGCCT | 1726 |
| 720 | ACACCUCA C UGGGGCUG | 602 | CAGCCCCA GGCTAGCTACAACGA TGAGGTGT | 1727 |
| 725 | UCAGUGGG C CUCUCGCA | 603 | TCCCACAG GGCTAGCTACAACGA CCCACTGA | 1728 |
| 728 | GUGGGGCU C UGGGAGAA | 604 | TTCTCCCA GGCTAGCTACAACGA AGCCCCAC | 1729 |
| 740 | GAGAAGAA A UCCGACCU | 704 | AGCTCGGA GGCTAGCTACAACGA TTCTTCTC | 1730 |
| 745 | GAAAUCCG A CCUUCGAU | 705 | ATCGAAGG GGCTAGCTACAACGA CGGATTTC | 1731 |
| 752 | GACCUCG A UGAACCGG | 706 | CCGGTTCA GGCTAGCTACAACGA CGAAGGTC | 1732 |
| 756 | UUCGAUGA A CCGGAAGC | 707 | GCTTCCGG GGCTAGCTACAACGA TCATCGAA | 1733 |
| 763 | AACCGGAA G CUGUCGGA | 605 | TCCGACAG GGCTAGCTACAACGA TTCCGGTT | 1734 |
| 766 | CCGAAGCU G UCGGAUCA | 606 | TGATCCGA GGCTAGCTACAACGA AGCTTCCG | 1735 |
| 771 | GCUGUCGG A UCACCCCC | 708 | GGGGGTGA GGCTAGCTACAAOGA CCGACAGC | 1736 |
| 774 | CUCGGAC A CCCCCCGA | 346 | TCGGGGGG GGCTAGCTACAACGA GATCCGAC | 1737 |
| 782 | ACCCCCCG A CCCUUCCC | 709 | GGGAAGGG GGCTAGCTACAACGA CGGGGGGT | 1738 |
| 793 | CUUCCCCU G CAGCAGCA | 520 | TGCTGCTG GGCTAGCTACAACGA AGGGGAAG | 1739 |
| 796 | CCCCUGCA G CAGCACCA | 607 | TGGTGCTG GGCTAGCTACAACGA TGCAGGGG | 1740 |
| 799 | CUGCAGCA G CACCAGCA | 608 | TGCTGGTG GGCTAGCTACAACGA TGCTGCAG | 1741 |
| 801 | GCAGCAGC A CCAGCACC | 361 | GGTGCTGG GGCTAGCTACAACGA GCTGCTGC | 1742 |
| 805 | CAGCACCA G CACCAGCC | 609 | GGCTGGTG GGCTAGCTACAACGA TGGTGCTG | 1743 |
| 807 | GCACCAGC A CCAGCCAC | 364 | GTGGCTGG GGCTAGCTACAACGA GCTGGTGC | 1744 |
| 811 | CAGCACCA G CCACAGCC | 610 | GGCTGTGG GGCTAGCTACAACGA TGGTGCTG | 1745 |
| 814 | CACCAGCC A CAGCCUCC | 368 | GGAGGCTG GGCTAGCTACAACGA GGCTGGTG | 1746 |
| 817 | CAGCCACA G CCUCCGCA | 611 | TGCGGAGG GGCTAGCTACAACGA TGTGGCTG | 1747 |
| 823 | CAGCCUCC G CAAUAUGC | 521 | GCATATTG GGCTAGCTACAACGA GGAGGCTG | 1748 |
| 826 | CCUCCGCA A UAUGCCCC | 710 | GGGGCATA GCCTAGCTACAACGA TGCGGAGG | 1749 |
| 828 | UCCGCAAU A UGCCCCAG | 139 | CTGGGGCA GGCTAGCTACAACGA ATTGCGGA | 1750 |
| 830 | CGCAAAU C CCCCAGCG | 522 | CGCTGCGG GGCTAGCTACAACGA ATATTGCG | 1751 |
| 836 | AUCCUCCA C CGCCCCAG | 612 | CTGGGCCG GGCTAGCTACAACGA IGUGGUAT | 1752 |
| 838 | GCCCCAUC C CCCCAGCA | 523 | TOCTUGGO GGCTAGCTACAACGA GOTUGGOC | 1753 |
| 844 | CUGOCOCA C CACCUCCA | 613 | TCCACCTG GCCTACCTACAACCA TGGGGCGC | 1754 |
| 847 | CCCCAGCA G CUGCAGCA | 614 | TUCTOCAC GGCTAGCTACAACCA TGCTCGCG | 1755 |
| 850 | CACCAGUD G CAGCAGCC | 524 | GGCTGCTG GGCTAGCTACAACGA AGCTGCTG | 1756 |
| 853 | CACCUGCA C CACCCCCC | 615 | GGGGGCTG GGCTAGCTACAACGA TGCACCTC | 1757 |
| 856 | CUGCAGCA C CCCCCACA | 616 | TGTGGGGG GCCTACCTACAACCA TGCTCCAG | 1758 |
| 862 | CACUCCUC A CACCACUC | 390 | CGCTGCTG CGCTAGCTACAACGA CCGGCCTG | 1759 |
| 865 | OCUCCACA G CAGCCAUA | 617 | TATCCCTC CGCTAGCTACAACCA TGTCGGGC | 1760 |
| 868 | OCACACCA C CGAUAUCU | 618 | ACATATCC CGCTACCTACAACCA TCCTGTGC | 1761 |
| 871 | CAGUACOC A UAUOUCCA | 711 | TOCACATA GCCTACCTACAACCA CCCTGCTC | 1762 |
| 873 | GCAGCCAU A UCUGCAGC | 140 | GCTCCAGA GGCTAGCTACAACGA ATCCCTCC | 1763 |
| 877 | CCAUAUCU G CACCACCA | 526 | TGGTGCTC CGCTAGCTACAACCA AGATATCC | 1764 |
| 880 | UAUCUGCA C CACCACCA | 619 | TGGTGGTC GGCTAGCTACAACGA TGCAGATA | 1765 |
| 882 | UCUGCAGC A CCACCAUU | 395 | AATCGTCG CCCTACCTACAACCA CCTGCACA | 1766 |
| 885 | GCAGCACC A CCAUUUCC | 397 | GCAAATCG GGCTAGCTACAACGA GGTGCTGC | 1767 |
| 888 | GCACCACC A UUUCCACC | 399 | CGTCCAAA CGCTAGCTACAACGA GCTGCTGC | 1768 |
| 894 | CCAUUUCC A CCAGGAAC | 401 | CTTCCTGC GGCTACCTACAACGA GGAAATGG | 1769 |
| 901 | CACCAGGA A CGCCGAGG | 712 | CCTCGGCG GGCTAGCTACAACGA TCCTGGTG | 1770 |
| 903 | CCAGGAAC G CCGAGCAG | 527 | CTCCTCGC CCCTACCTACAACCA GTTCCTCC | 1771 |
| 912 | CCGACCAG C CACCCUUG | 620 | CAAGCCTG GGCTAGCTACAACGA CTCCTCGG | 1772 |
| 915 | ACGACGCA C CCUUCACA | 621 | TGTCAACG GCCTACCTACAACGA TCCCTCCT | 1773 |
| 921 | CACCC3UC A CAUAAAUG | 713 | CATTTATG GGCTACCTACAACGA CAAGGCTG | 1774 |
| 923 | GCCUUGAC A UAAAUGAU | 408 | ATCATTTA GGCTAGCTACAACGA GTCAACGC | 1775 |
| 927 | UGACAUAA A UGAUCGGC | 714 | CCCCATCA GGCTAGCTACAACCA TTATGTCA | 1776 |
| 930 | CAUAAAUG A UGGGCAUU | 715 | AATCCCCA GGCTAGCTACAACCA CATTTATG | 1777 |
| 934 | AAUCAUGG G CAUUGUCC | 622 | CCACAATC CGCTAGCTACAACCA CCATCATT | 1778 |
| 936 | UCAUGGCC A UUCUCCCA | 409 | TGCCACAA CCCTAGCTACAACCA GCCCATCA | 1779 |
| 939 | UGCAGCA3 C UGGCACCG | 623 | CGGTGCCA CGCTAGCTACAACGA AATGCCCA | 1780 |
| 942 | CCAUUGUG C CACCCCCU | 624 | AGCCCGTC CCCTACCTACAACGA CACAATGC | 1781 |
| 944 | AUUGUCGC A CCGCCUUC | 410 | CAAGCCGG GGCTAGCTACAACGA GCCACAAT | 1782 |
| 949 | UCGCACCG G CUUGGGCA | 625 | TGCCCAAC GGCTAGCTACAACGA CCCTCCCA | 1783 |
| 954 | CGGCUUCG G CAGUGAAA | 626 | TTTCACTC CGCTACCTACAACGA COAACCCG | 1784 |
| 957 | CUUGGGCA C UGAAAUCA | 627 | TCATTTCA GGCTACCTACAACGA TGCCCAAC | 1785 |
| 962 | GCAGUCAA A UGAAUGCG | 716 | CCCATTCA GGCTACCPACAACCA TTCAOTCC | 1786 |
| 966 | UGAAAUGA A UCCCGCCC | 717 | CCGCCGCA GGCTAGCTACAACGA TCATTTCA | 1797 |
| 968 | AAAUGAAU G CGGCCCUC | 533 | GAGCGCCC CGCTAGCTACAACGA ATTCATTT | 1788 |
| 971 | UCAAUGCC G CCCUCAUG | 628 | CATGAGGG GCCTAGCTACAACGA CCCATTCA | 1789 |
| 977 | CGCCCCUC A UGCAUCGC | 417 | CCCATCCA CCCTAGCTACAACCA CAGCGCCC | 1790 |
| 979 | GCCCUCAU G CAUCGCAC | 534 | CTCCGATG GGCTACCTACAACGA ATCACCCC | 1791 |
| 981 | CCUCAUGC A UCGGAGAC | 418 | GTCTCCGA CGCTAGCTACAACGA CCATGACG | 1792 |
| 988 | CAUCGCAG A CACACAGA | 718 | TCTGTGTG CGCTAGCTACAACCA CTCCGATC | 1793 |
| 990 | UCGGAGAC A CACAGACC | 419 | GGTCTGTC GGCTACCTACAACGA CTCTCCGA | 1794 |
| 992 | GGAGACAC A CAGACCCA | 420 | TGGCTCTG GGCTACCTACAACCA GTGTCTCC | 1795 |
| 996 | ACACACAC A CCCAGUGC | 719 | CCACTCGC GGCTAGCTACAACGA CTGTGTGT | 1796 |
| 1001 | CAGACCCA G UGCAGCUC | 629 | CACCTCCA CGCTAGCTACAACGA TCCGTCTC | 1797 |
| 1003 | GACCCAGU C CAGCUCCA | 535 | TCCACCTC GCCTACCTACAACCA ACTCCCTC | 1798 |
| 1006 | CCAGUGCA G CUCCAGUC | 630 | CCCTCCAG GCCTACCTACAACGA TGCACTGG | 1799 |
| 1013 | AGCUCCAG G CGGCAGCC | 631 | CCCTCCCG GGCTAGCTACAACGA CTGGACCT | 1800 |
| 1016 | UCCAGGCG G CAGGUCCA | 632 | TCGCCCTG GGCTAGCTACAACGA CGCCTCGA | 1801 |
| 1021 | CCGGCAGC G CGAGUGCG | 633 | CGCACTCG GCCTAGCTACAACCA CCTGCCGC | 1802 |
| 1025 | CACGGCGA G UGCGGUGG | 634 | CCACCGCA GGCTAGCTACAACGA TCGCCCTG | 1803 |
| 1027 | GGCCGACU C CGCUCCGC | 537 | CCCCACCC GCCTACCTACAACCA ACTCCCCC | 1804 |
| 1030 | CGAGUCCG C UGGGCCCG | 635 | CGGGCCCA CCCTACCTACAACGA CCCACTCC | 1805 |
| 1034 | UCCCCUCC C CCCCGCCC | 636 | CGCCCGGG CCCTACCTACAACCA CCACCCCA | 1806 |
| 1040 | CCCCCCCC C CCCUCUAU | 637 | ATACAGCG CCCTACCTACAACCA CCCCCCCC | 1807 |
| 1042 | CCCCCGCC C CUCUAUCA | 538 | TCATACAG CCCTACCTACAACCA CCCCCCCC | 1808 |
| 1045 | CGGGCCCU C UAUCACUU | 638 | AACTCATA CCCTACCTACAACCA ACCCCCCC | 1809 |
| 1047 | CCCCCUCU A UCACUUUC | 152 | CAAACTCA CCCTACCTACAACCA ACACCCCC | 1810 |
| 1050 | CCUCUAUC A CUUUCACC | 720 | CCTCAAAC CCCTACCTACAACCA CATACAGC | 1811 |
| 1058 | ACUUUGAG C CCCUGGAG | 639 | CTCCAGGG CCCTACCTACAACCA CTCAAACT | 1812 |
| 1068 | CCUGCAGC A UGACGAGC | 721 | CCTCGTCA CCCTACCTACAACCA CCTCCAGC | 1813 |
| 1071 | GGAGGAUG A CGAGCUGG | 722 | CCACCTCC CCCTACCTACAACCA CATCCTCC | 1814 |
| 1075 | GAUCACCA C CUCCUCUC | 640 | AACCCCAC CCCTACCTACAACCA TCCTCATC | 1815 |
| 1081 | GAGCUGGG C UUCCACAG | 641 | CTCTCCAA CCCTACCTACAACCA CCCACCTC | 1816 |
| 1086 | GGGGUUCC A CACCCCCG | 439 | CCCCCCTC CCCTACCTACAACCA CCAACCCC | 1817 |
| 1089 | GUUCCACA C CCCCCAGC | 642 | CCTCCCCC CCCTACCTACAACCA TCTCCAAC | 1818 |
| 1097 | GCGCCCAC C UCCUGGAC | 643 | CTCCACCA CCCTACCTACAACCA CTCCCCCC | 1819 |
| 1100 | GGGGGCUG C UGGAGGUC | 644 | CACCTCCA CCCTACCTACAACCA CACCTCCC | 1820 |
| 1106 | UCCUCCAG C UCCUGGAU | 645 | ATCCACCA CCCTACCTACAACCA CTCCACCA | 1821 |
| 1113 | GGUGGUCC A UACCUGGA | 723 | TUCACOTA CCCTACCTACAACCA CCAGCACC | 1822 |
| 1116 | CCUCCAUA C CUCCAACC | 646 | CCTTCCAC CCCTACCTACAACCA TATCCACC | 1823 |
| 1122 | UAGCUCCA A CCCAUCCU | 724 | ACCATCCC CCCTACCTACAACCA TGCACCTA | 1824 |
| 1126 | UCCAACCC A UCCUGGUG | 448 | CACCACCA CGCTAGCTACAACGA CCGTTCCA | 1825 |
| 1132 | CCAUCCUG G UGGACCGG | 647 | CCCCTCCA CCCTACCTACAACCA CACCATUG | 1826 |
| 1136 | CCUGGUGG A CCGGCCGC | 725 | CCCGCCCG CCCTACCTACAACCA CCACCACC | 1827 |
| 1140 | GUCCACCG C CCGCCUGC | 648 | CCACCCCC CCCTACCTACAACCA CCCTCCAC | 1828 |
| 1143 | GACCGGCC G CCUGCACA | 543 | TGTGCAGG GGCTAGCTACAACGA GGCCGGTC | 1829 |
| 1147 | GGCCCCCU C CACAACAA | 544 | TTCTTCTC CGCTAGCTACAACGA ACCCCCCC | 1830 |
| 1149 | CCGCCCGC A CAACAAGC | 455 | CCTTCTTG GGCTAGCTACAACCA GCAGGCGG | 1831 |
| 1152 | CCUGCACA A CAACCUGG | 726 | CCAGCTTG GGCTAGCTACAACGA TGTGCAGG | 1832 |
| 1156 | CACAACAA C CUGGGCCU | 649 | AGGCCCAG GGCTAGCTACAACGA TTCTTGTG | 1833 |
| 1161 | CAACCUCC C CCJCUUCC | 650 | GGAACACG GCCTAGCTACAACGA CCACCTTC | 1834 |
| 1172 | UCUUCCCU C CCAACUAC | 545 | GTAGTTGG GCCTACCTACAACCA ACCCAACA | 1835 |
| 1176 | CCCUCCCA A CUACGUGC | 727 | CCACCTAG CGCTAGCTACAACGA TGCCAGGG | 1836 |
| 1179 | UCCCAACU A CCUCCCAC | 164 | GTCCCACC CCCTAGCTACAACGA AGTTCCCA | 1837 |
| 1181 | CCAACUAC G UCCCACCC | 651 | CGCTCCCA CCCTACCTACAACCA CTAGTTGG | 1838 |
| 1184 | ACLACGDC G CACCCAUG | 652 | CATGCGTG CGCTACCTACAACCA CACCTAGT | 1839 |
| 1186 | UACCUGCC A CCCAUCAC | 468 | GTCATGGG GCCTAGCTACAACGA CCCACGTA | 1840 |
| 1190 | UCGCACCC A UCACCCCA | 471 | TCGCGTCA GGCTAGCTACAACCA CGCTCCCA | 1841 |
| 1193 | CACCCAUG A CCCGAUAA | 728 | TTATCGCC CGCTACCTACAACGA CATCCCTC | 1842 |
| 1198 | AUCACCCC A UAAACUCU | 729 | AGACTTTA GGCTAGCTACAACCA CGGGTCAT | 1843 |
| 1202 | CCCGAUAA A CUCUUCAC | 730 | CTGAAGAC GCCTAGCTACAACGA TTATCGGC | 1844 |
| 1214 | UUCAGGCC A CAGAAGCU | 731 | AGCTTCTG CCCTAGCTACAACGA CCCCTGAA | 1845 |
| 1220 | GGACAGAA G CUUUUUGU | 653 | ACAAAAAC GGCTAGCTACAACCA TTCTCTCC | 1846 |
| 1227 | AGCUUUUU C UCUGGAGC | 654 | COTOCAGA GGCTAGCTACAACCA AAAAACCT | 1847 |
| 1234 | UGUCUGGA G CUGCCCAC | 655 | CTCGGCAG GGCTAGCTACAACCA TCCAGACA | 1848 |
| 1237 | CUGCAGCU G CCCACAAG | 548 | CTTGTCGC GGCTAGCTACAACGA AGCTCCAG | 1849 |
| 1241 | AGCUGCCC A CAAGAAAG | 483 | CTTTCTTG GGCTAGCTACAACGA GGGCAGCT | 1850 |
| 1253 | CAAAGAGG G CAAGGAAA | 656 | TTTCCTTG GGCTAGCTACAACCA CCTCTTTC | 1851 |
| 1266 | GAAAAAAG C CUGGACUC | 657 | CAGTCCAG GCCTAGCTACAACGA CTTTTTTC | 1852 |
| 1271 | AAGGCUCG A CUCCAUGA | 732 | TCATCCAC CCCTACCTACAACCA CCAGCCTT | 1853 |
| 1276 | UGGACUCC A UGACUAUA | 489 | TATACTCA CCCTACCTACAACCA CCACTCCA | 1854 |
| 1279 | ACUCCAUG A CUAUAUAU | 733 | ATATATAG CGCTACCTACAACCA CATGCAGT | 1855 |
| 1282 | CCAUGACU A UAUAUACA | 175 | TCTATATA CCCTACCTACAACGA AGTCATCG | 1856 |
| 1284 | AUGACUAU A UAUACAUA | 176 | TATCTATA CCCTACCTACAACGA ATACTCAT | 1857 |
| 1286 | GACUAUAU A UACAUACA | 177 | TCTATGTA GCCTAGCTACAACCA ATATACTC | 1858 |
| 1288 | CUAUAUAU A CAUACAUC | 178 | CATGTATG CCCTACCTACAACCA ATATATAC | 1859 |
| 1290 | AUAUAUAC A UACAUCUA | 491 | TACATCTA CGCTACCTACAACCA CTATATAT | 1860 |
| 1292 | AUAUACAU A CAUCUAUC | 179 | CATACATC CCCTACCTACAACCA ATCTATAT | 1861 |
| 1294 | AUACAUAC A UCUAUCUA | 492 | TACATACA CCCTACCTACAACCA CTATCTAT | 1862 |

Input Sequence = HSA011736.
Cut Site = R/Y
Stem Length = 8.
Core Sequence = GGCTAGCTACAACGA
HSA011736 (Home sapiens mRNA for growth factor receptor binding protein (GRBLG); 1303 bp)

TABLE VIII

| Human GRID Amberzyme and Substrate Sequence | | | | |
|---|---|---|---|---|
| Pos | Substrate | Seq ID | Amberzyme | Seq ID |
| 11 | GAGGGACA G UUAAUGGA | 550 | UCCAUUAA GGAGGAAACUCC CU UCAAGGACAUCGUCCGGG UGUGCCUC | 1863 |
| 17 | CAGUUAAU G GAUCCUGA | 734 | GACAGAUC GGAGGAAACUCC CU UCAAGGACAUCGUCCGGG AUUAACUG | 1864 |
| 18 | AGUUAAUG G AUCUGUAA | 735 | UUACAGAU GGAGGAAACUCC CU UCAAGGACAUCGUCCGGG CAUUAACU | 1865 |
| 23 | AUGGAUCU G UAAACUUG | 551 | CAAGUUUA GGAGGAAACUCC CU UCAAGGACAUCGUCCGGG AGAUCCAU | 1866 |
| 31 | GUAAACUU G CACCCUCU | 493 | AGAGGGUG GGAGGAAACUCC CU UCAAGGACAUCGUCCGGG AAGUUUAC | 1867 |
| 44 | CUCUUUCA G AGUGGUAC | 736 | GUACCACU GGAGGAAACUCC CU UCAAGGACAUCGUCCGGG UGAAAGAG | 1868 |
| 46 | CUUUCAGA G UGGUACAU | 552 | AUGUACCA GGAGGAAACUCC CU UCAAGGACAUCGUCCGGG UCUGAAAG | 1869 |
| 48 | UUCAGAGU G GUACAUGG | 737 | CCAUGUAC GGAGGAAACUCC CU UCAAGGACAUCGUCCGGG ACUCUGAA | 1870 |
| 49 | UCAGAGUG G UACAUGGA | 553 | UCCAUGUA GGAGGAAACUCC CU UCAAGGACAUCGUCCGGG CACUCUCA | 1871 |
| 55 | UGGUACAG G GAAGACAG | 738 | CUGUCUUC GGAGGAAACUCC CU UCAAGGACAUCGUCCGGG AUGUACCA | 1872 |
| 56 | GGUACAUG G AAGACAGC | 739 | GCUGUCUU GGAGGAAACUCC CU UCAAGGACAUCGUCCGGG CAUGUACC | 1873 |
| 59 | ACAUGGAA G ACAGCACA | 740 | UGUGCUGU GGAGGAAACUCC CU UCAAGGACAUCGUCCGGG UUCCAUGU | 1874 |
| 63 | GCAAGACA G CACAAACU | 554 | ACUGUGUG GGAGGAAACUCC CU UCAAGGACAUCGUCCGGG UGUCUUCC | 1875 |
| 70 | ACCACASA G UGGAUCCA | 555 | UGGAUCCA GGAGGAAACUCC CU UCAAGGACAUCGUCCGGG UUUGUGCU | 1876 |
| 72 | CACAAACU G CAUCCAUA | 741 | GAUGGAUC GGAGGAAACUCC CU UCAAGGACAUCGUCCGGG ACUUUGUG | 1877 |
| 73 | ACAAACUC G AUCCAUAC | 742 | GUAUGCAU GGAGGAAACUCC CU UCAAGGACAUCGUCCGGG CACUUUGU | 1878 |
| 85 | CAUACUCU G AAAUGCAG | 494 | CUGCAUUU GGAGGAAACUCC CU UCAAGGACAUCGUCCGGG ACAGUAUG | 1879 |
| 90 | UCUGAAAU G CAGUAACU | 495 | AGGUACUC GGAGGAAACUCC CU UCAAGGACAUCGUCCGGG AUUUCAGA | 1880 |
| 93 | CAAAUCCA G UAACUCUG | 556 | CAGACUUA GGAGGAAACUCC CU UCAAGGACAUCGUCCGGG UGCAUUUC | 1861 |
| 101 | GUAACUCU G AUGCUUGA | 496 | UCAACCAU GGAGGAAACUCC CU UCAAGGACAUCGUCCGGG AGAGUUAC | 1882 |
| 104 | ACUCUGAU G CUUGAAUU | 497 | AAUUCAAG GGAGGAAACUCC CU UCAAGGACAUCGUCCGGG AUCAGAGU | 1883 |
| 108 | UGAUGCUU G AAUUUCUU | 498 | AACAAAUU GGAGGAAACUCC CU UCAAGGACAUCGUCCGGG AAGCAUCA | 1884 |
| 114 | UUGAAUUU G UUCUCCCU | 557 | AGCGACAA GGAGGAAACUCC CU UCAAGGACAUCGUCCGGG AAAUUCAA | 1885 |
| 127 | CCCUUCUU G CCAGAAAG | 499 | CUUUCUGG GGAGGAAACUCC CU UCAAGGACAUCGUCCGGG AAGAAGGG | 1886 |
| 131 | UCUUGCCA G AAAGGAUU | 743 | AAUCCUUU GGAGGAAACUCC CU UCAAGGACAUCGUCCGGG UGGCAAGA | 1887 |
| 135 | GCCAGAAA G CAUUCUAA | 744 | UUACAAUC GGAGGAAACUCC CU UCAAGGACAUCGUCCGGG UUUCUCCC | 1888 |
| 136 | CCACAAAG G AUUCUAAU | 745 | AUUAGAAU GGAGGAAACUCC CU UCAAGGACAUCGUCCGGG CUUUCUCC | 1889 |
| 150 | AAUAACUC G CUCUCAAA | 746 | UUUGACAC GGAGGAAACUCC CU UCAAGGACAUCGUCCGGG CAGUUAUU | 1890 |
| 151 | AUAACUCC G UCUCAAAC | 558 | CUUUGACA GGAGGAAACUCC CU UCAAGGACAUCGUCCGGG CCAGUUAU | 1891 |
| 153 | AACUCCCU G UCAAACCC | 559 | CCCUUUGA GGAGGAAACUCC CU UCAAGGACAUCGUCCGGG ACCCACUU | 1892 |
| 159 | GUGUCAAA G CCAAGACA | 560 | UCUCUUCC GGAGGAAACUCC CU UCAAGGACAUCGUCCGGG UUUCACAC | 1893 |
| 164 | AAAGCCAA G ACAUAAAC | 747 | GUUUAUCU GGAGGAAACUCC CU UCAAGGACAUCGUCCGGG UUCCCUUU | 1894 |
| 194 | UUCCAAAA G CUUCACGU | 561 | ACGUGAAG GGAGGAAACUCC CU UCAAGGACAUCGUCCGGG UUUUGGAA | 1895 |
| 201 | AGCCUCAC G UUACAGCA | 562 | UGCUGUAA GGAGGAAACUCC CU UCAAGGACAUCGUCCGGG CUGAAGCU | 1896 |
| 207 | ACGUUACA G CAUGGAAG | 563 | CUUCCAUG GGAGGAAACUCC CU UCAAGGACAUCGUCCGGG UGUAACGU | 1897 |
| 211 | UACAGCAU G GAAGCUGU | 748 | ACAGCUUC GGAGGAAACUCC CU UCAAGGACAUCGUCCGGG AUGCUGUA | 1898 |
| 212 | ACAGCAUG G AAGCUGUU | 749 | AACAGCUU GGAGGAAACUCC CU UCAAGGACAUCGUCCGGG CAUGCUGU | 1899 |
| 215 | GCAUGGAA G CUGUUGCC | 564 | GGCAACAG GGAGGAAACUCC CU UCAAGGACAUCGUCCGGG UUCCAUGC | 1900 |
| 218 | UGGAAGCU G UUGCCAAG | 565 | CUUGGCAA GGAGGAAACUCC CU UCAAGGACAUCGUCCGGG AGCUUCCA | 1901 |
| 221 | AACCUGUU G CCAAGUUU | 500 | AAACUUGG GGAGGAAACUCC CU UCAAGGACAUCGUCCGGG AACAGCUU | 1902 |
| 226 | GUUGCCAA G UUUGAUUU | 586 | AAAUCAAA GGAGGAAACUCC CU UCAAGGACAUCGUCCGGG UUGGCAAC | 1903 |
| 230 | CCAACUUU G AUUUCACU | 501 | ACUGAAAU GGAGGAAACUCC CU UCAAGGACAUCGUCCGGG AAACUUGC | 1904 |
| 239 | AUUUCACU G CUUCAGGU | 502 | ACCUGAAG GGAGGAAACUCC CU UCAAGGACAUCGUCCGGG AGUGAAAU | 1905 |
| 245 | CUGCUUCA G GUGAGGAU | 750 | AUCCUCAC GGAGGAAACUCC CU UCAAGGACAUCGUCCGGG UGAACCAG | 1906 |
| 246 | UCCUUCAC G UCAGGAUG | 567 | CAUCCUCA GGAGGAAACUCC CU UCAAGGACAUCGUCCGGG CUGAAGCA | 1907 |
| 248 | CUUCAGCU G AGGAUGAA | 503 | UUCAUCCU GGAGGAAACUCC CU UCAAGGACAUCGUCCGGG ACCUCAAG | 1908 |
| 250 | UCAGGUCA G CAUCAACU | 751 | ACUUCAUC GGAGGAAACUCC CU UCAAGGACAUCGUCCGGG UCACCUCA | 1909 |
| 251 | CAGGUCAC G AUCAACUC | 752 | CACUUCAU GGAGGAAACUCC CU UCAAGGACAUCGUCCGGG CUCACCUG | 1910 |
| 254 | GUGACCAU G AACUCACC | 504 | CCUCACUU GGAGGAAACUCC CU UCAAGGACAUCGUCCGGG AUCCUCAC | 1911 |
| 259 | GAUCAACU G ACCUUUCA | 505 | UCAAACCU GGAGGAAACUCC CU UCAAGGACAUCGUCCGGG ACUUCAUC | 1912 |
| 261 | UGAACUCA G CUUUCACA | 568 | UGUGAAAC GGAGGAAACUCC CU UCAAGGACAUCGUCCGGG UCACUUCA | 1913 |
| 272 | UUCACACU G GACAUCUU | 753 | AACAUCUC GGAGGAAACUCC CU UCAAGGACAUCGUCCGGG ACUCUGAA | 1914 |
| 273 | UCACACUC G ACAUCUUU | 754 | AAACAUCU GGAGGAAACUCC CU UCAAGGACAUCGUCCGGG CACUGUGA | 1915 |
| 275 | ACACUCCA G AUCUUUUG | 755 | CAAAACAU GGAGGAAACUCC CU UCAAGGACAUCGUCCGGG UCCAGUGU | 1916 |
| 278 | CUCCACAU G UUUUCAAG | 569 | CUUCAAAA GGAGGAAACUCC CU UCAAGGACAUCGUCCGGG AUCUCCAG | 1917 |
| 283 | CAUCUUUU G AACAUUUU | 506 | AAAAUCUU GGAGGAAACUCC CU UCAAGGACAUCGUCCGGG AAAACAUC | 1918 |
| 286 | GUUUUCAA G AUUUUAAC | 756 | CUUAAAAU GGAGGAAACUCC CU UCAAGGACAUCGUCCGGG UUCAAAAC | 1919 |
| 294 | GAUUUUAA G UAACCAAC | 570 | CUUCGUUA GGAGGAAACUCC CU UCAAGGACAUCGUCCGGG UUAAAAUC | 1920 |
| 302 | CUAACCAA G AGGACUCC | 757 | CCACUCCU GGAGGAAACUCC CU UCAAGGACAUCGUCCGGG UUCGUUAC | 1921 |
| 304 | AACCAACA G CACUGCUU | 758 | AACCACUC GGAGGAAACUCC CU UCAAGGACAUCGUCCGGG UCUUCCUU | 1922 |
| 305 | ACCAAGAC G ACUCGUUU | 759 | AAACCACU GGAGGAAACUCC CU UCAAGGACAUCGUCCGGG CUCUUCCU | 1923 |
| 307 | CAACACGA G UCCUUUAA | 571 | UUAAACCA GGAGGAAACUCC CU UCAAGGACAUCGUCCGGG UCCUCUUC | 1924 |
| 309 | ACACCACU G CUUUAAGG | 760 | CCUUAAAC GGAGGAAACUCC CU UCAAGGACAUCGUCCGGG ACUCCUCU | 1925 |
| 310 | GACCACUC G UUUAACCC | 572 | GCCUUAAA GGAGGAAACUCC CU UCAAGGACAUCGUCCGGG CACUCCUC | 1926 |
| 316 | UGGUUUAA G GCGCACCU | 761 | ACCUCCCC GGAGGAAACUCC CU UCAAGGACAUCGUCCGGG UUAAACCA | 1927 |
| 317 | CCUUUAAC G CCCACCUU | 573 | AAGCUCCC GGAGGAAACUCC CU UCAAGGACAUCGUCCGGG CUUAAACC | 1928 |
| 319 | UUUAAGGC G GAGCUUGG | 762 | CCAAGCUC GGAGGAAACUCC CU UCAAGGACAUCGUCCGGG GCCUUAAA | 1929 |
| 320 | UUAAGGCG G AGCUUGCG | 763 | CCCAAGCU GGAGGAAACUCC CU UCAAGGACAUCGUCCGGG CGCCUUAA | 1930 |
| 322 | AACCCGGA G CUUCCGAG | 574 | CUCCCAAG GGAGGAAACUCC CU UCAAGGACAUCGUCCGGG UCCGCCUU | 1931 |
| 326 | CUGACCUC G GGAGCCAG | 764 | CUGGCUCC GGAGGAAACUCC CU UCAAGGACAUCGUCCGGG AACCUCCG | 1932 |
| 327 | GGAGCUUG G GAGCCAGG | 765 | CCUCGCUC GGAGGAAACUCC CU UCAAGGACAUCGUCCGGG CAAGCUCC | 1933 |
| 328 | GAGCUUGC G AGCCAGGA | 766 | UCCUGGCU GGAGGAAACUCC CU UCAAGGACAUCGUCCGGG CCAAGCUC | 1934 |
| 330 | GCUUGCGA G CCACGAAG | 575 | CUUCCUGG GGAGGAAACUCC CU UCAAGGACAUCGUCCGGG UCCCAAGC | 1935 |
| 334 | GGGACCCA G GAAGGAUA | 767 | UAUCCUUC GGAGGAAACUCC CU UCAAGGACAUCGUCCGGG UGUCUCOC | 1936 |
| 335 | GGACCCAC G AAGCAUAU | 768 | AUAUCCUU GGAGGAAACUCC CU UCAAGGACAUCGUCCGGG CUCGCUCC | 1937 |
| 338 | GCCACGAA G GAUAUCUC | 769 | CACAUAUC GGAGGAAACUCC CU UCAAGGACAUCGUCCGGG UUCCUCCC | 1938 |
| 339 | CCAGGAAG G AUAUGUGC | 770 | GCACAUAU GGAGGAAACUCC CU UCAAGGACAUCGUCCGGG CUUCCUCG | 1939 |
| 344 | AAGGAUAU G UGCCCAAC | 576 | CUUCGCCA GGAGGAAACUCC CU UCAAGGACAUCGUCCGGG AUAUCCUU | 1940 |
| 346 | GGAUAUGU G CCCAACAA | 507 | UUCUUGGG GGAGGAAACUCC CU UCAAGGACAUCGUCCGGG ACAUAUCC | 1941 |
| 352 | GUGCCCAA G AAUUUCAU | 771 | AUCASAUU GGAGGAAACUCC CU UCAAGGACAUCGUCCGGG UUGGCCAC | 1942 |
| 362 | AUUUCAUA G ACAUCCAG | 772 | CUCGAUGU GGAGGAAACUCC CU UCAAGGACAUCGUCCGGG UAUGAAAU | 1943 |
| 370 | GACAUCCA G UUUCCCAA | 577 | UUGCGAAA GGAGGAAACUCC CU UCAAGGACAUCGUCCGGG UGGAUCUC | 1944 |
| 391 | UCCCAAAU G GUUUCACC | 773 | CGUCAAAC GGAGGAAACUCC CU UCAAGGACAUCGUCCGGG AUUUGGCA | 1945 |
| 382 | CCCAAAUG G UUUCACGA | 578 | UCGUGAAA GGAGGAAACUCC CU UCAAGGACAUCGUCCGGG CAUUUCCC | 1946 |
| 389 | GGUUUCAC G AAGGCCUC | 508 | GAGGCCUU GGAGGAAACUCC CU UCAAGGACAUCGUCCGGG GUGAAACC | 1947 |
| 392 | UUCACGAA G GCCUCUCU | 774 | AGAGACGC GGAGGAAACUCC CU UCAAGGACAUCGUCCGGG UUCCUCAA | 1948 |
| 393 | UCACGAAG G CCUCUCUC | 579 | GAGAGACC GGAGGAAACUCC CU UCAAGGACAUCGUCCGGG CUUCCUCA | 1949 |
| 402 | CCUCUCUC G ACACCAGG | 509 | CCUCCUCU GGAGGAAACUCC CU UCAAGGACAUCGUCCGGG CACACACC | 1950 |
| 409 | CGACACCA G GCAGAGAA | 775 | UUCUCUCC GGAGGAAACUCC CU UCAAGGACAUCGUCCGGG UCCUCUCC | 1951 |
| 410 | GACACCAG G GAGAGAAC | 580 | COUCUCUC GGAGGAAACUCC CU UCAAGGACAUCGUCCGGG CUCCUCUC | 1952 |
| 413 | ACCAGGCA G AGAACUUA | 776 | UAACUUCU GGAGGAAACUCC CU UCAAGGACAUCGUCCGGG UCCCUCCU | 1953 |
| 415 | CAGGCAGA G AACUUACU | 777 | ACUAACUU GGAGGAAACUCC CU UCAAGGACAUCGUCCGGG UCUCCCUC | 1954 |
| 427 | UUACUCAU G GGCAAGGA | 778 | UCCUUCCC GGAGGAAACUCC CU UCAAGGACAUCGUCCGGG AUCACUAA | 1955 |
| 428 | UACUCAUG G GCAAGGAG | 779 | CUCCUUCC GGAGGAAACUCC CU UCAAGGACAUCGUCCGGG CAUCACUA | 1956 |
| 429 | ACUCAUGG G CAAGGAGG | 581 | CCUCCUUC GGAGGAAACUCC CU UCAAGGACAUCGUCCGGG CCAUCACU | 1957 |
| 433 | AUGGGCAA G GAGGUUGG | 780 | CCAACCUC GGAGGAAACUCC CU UCAAGGACAUCGUCCGGG UUGCCCAU | 1958 |
| 434 | UGGGCAAG G AGGUUGGC | 781 | GCCAACCU GGAGGAAACUCC CU UCAAGGACAUCGUCCGGG CUUGCCCA | 1959 |
| 436 | GGCAAGGA G GUUGGCUU | 782 | AACCCAAC GGAGGAAACUCC CU UCAAGGACAUCGUCCGGG UCCUUCCC | 1960 |
| 437 | GCAAGGAG G UUGGCUUC | 582 | CAACCCAA GGAGGAAACUCC CU UCAAGGACAUCGUCCGGG CUCCUUGC | 1961 |
| 440 | AGGAGCUU G GCUUCUUC | 783 | CAACAACC GGAGGAAACUCC CU UCAAGGACAUCGUCCGGG AACCUCCU | 1962 |
| 441 | GGAGCUUG G CUUCUUCA | 593 | UGAAGAAG GGAGGAAACUCC CU UCAAGGACAUCGUCCGGG CAACCUCC | 1963 |
| 456 | CAUCAUCC G GGCCAGCC | 784 | GGCUGGCC GGAGGAAACUCC CU UCAAGGACAUCGUCCGGG GGAUGAUG | 1964 |
| 457 | AUCAUCCG G GCCAGCCA | 785 | UGGCUGGC GGAGGAAACUCC CU UCAAGGACAUCGUCCGGG CCGAUGAU | 1965 |
| 458 | UCAUCCGC G CCAGCCAG | 584 | CUCGCUGG GGAGGAAACUCC CU UCAAGGACAUCGUCCGGG CCGGAUGA | 1966 |
| 462 | CCGGGCCA G CCACAGCU | 565 | AGCUCUGG GGAGGAAACUCC CU UCAAGGACAUCGUCCGGG UGGCCCGG | 1967 |
| 466 | GCCAGCCA G AGCUCCCC | 786 | GGGGAGCU GGAGGAAACUCC CU UCAAGGACAUCGUCCGGG UGGCUGGC | 1968 |
| 468 | CAGCCACA G CUCOCCAG | 586 | CUGGGCAG GGAGGAAACUCC CU UCAAGGACAUCGUCCGGG UCUGGCUC | 1969 |
| 476 | GCUCCCCA G GGGACUUC | 787 | GAAGUCCC GGAGGAAACUCC CU UCAAGGACAUCGUCCGGG UGGGGACC | 1970 |
| 477 | CUCCCCAG G GGACUUCU | 788 | ACAAGUCC GGAGGAAACUCC CU UCAAGGACAUCGUCCGGG CUGGGCAG | 1971 |
| 478 | UCCCCAGG G GACUUCUC | 789 | GAGAAGUC GGAGGAAACUCC CU UCAAGGACAUCGUCCGGG CCUGCGCA | 1972 |
| 479 | CCCCAGGC G ACUUCUCC | 790 | GGAGAAGU GGAGGAAACUCC CU UCAAGGACAUCGUCCGGG CCCUGCGG | 1973 |
| 494 | CCAUCUCU G UCAGGCAU | 587 | AUGCCUGA GGAGGAAACUCC CU UCAAGGACAUCGUCCGGG AGACAUCG | 1974 |
| 498 | CUCUGUCA G GCAUGACG | 791 | CCUCAUCC GGAGGAAACUCC CU UCAAGGACAUCGUCCGGG UCACAGAG | 1975 |
| 499 | UCUGUCAG G CAUCAGGA | 598 | UCCUCAUG GGAGGAAACUCC CU UCAAGGACAUCGUCCGGG CUCACAGA | 1976 |
| 503 | UCAGCCAU G AGGAUGAC | 510 | GUCAUCCU GGAGGAAACUCC CU UCAAGGACAUCGUCCGGG AUGCCUGA | 1977 |
| 505 | AGOCAUCA G GAUGACGU | 792 | ACCUCAUC GGAGGAAACUCC CU UCAAGGACAUCGUCCGGG UCAUGCCU | 1979 |
| 506 | CCCAUGAC G AUCACGUU | 793 | AACGGUCA GGAGGAAACUCC CU UCAAGGACAUCGUCCGGG CUCAUGCC | 1979 |
| 509 | AUGAGGAU G ACGUUCAA | 511 | UUCAACCU GGAGGAAACUCC CU UCAAGGACAUCGUCCGGG AUCCUCAU | 1988 |
| 512 | ACCAUGAC G UUCAACAC | 589 | GUGUUCAA GGAGGAAACUCC CU UCAAGGACAUCGUCCGGG GUCAUCCU | 1981 |
| 526 | CACUUCAA G GUCAUGCG | 794 | CGCAUCAC GGAGGAAACUCC CU UCAAGGACAUCGUCCGGG UUCAACUC | 1982 |
| 527 | ACUUCAAC G UCAUGCGA | 598 | UCCCAUCA GGAGGAAACUCC CU UCAAGGACAUCGUCCGGG CUUCAACU | 1983 |
| 532 | AAGGUCAU G CGACACAA | 512 | UUCUCUCG GGAGGAAACUCC CU UCAAGGACAUCGUCCGGG AUCACCUU | 1984 |
| 534 | GGUCAUGC G AGACAACA | 513 | UCUUCUCU GGAGGAAACUCC CU UCAAGGACAUCGUCCGGG CCAUCACC | 1985 |
| 536 | UCAUGCGA G ACAACAAG | 795 | CUUCUUCU GGAGGAAACUCC CU UCAAGGACAUCGUCCGGG UCCCAUCA | 1986 |
| 544 | GACAACAA G GGUAAUUA | 796 | UAAUUACC GGAGGAAACUCC CU UCAAGGACAUCGUCCGGG UUCUUCUC | 1987 |
| 545 | ACAACAAG G GUAAUUAC | 797 | CUAAUUAC GGAGGAAACUCC CU UCAAGGACAUCGUCCGGG CUUCUUCU | 1988 |
| 546 | CAACAACG G UAAUUACU | 591 | AGUAUUAG GGAGGAAACUCC CU UCAAGGACAUCGUCCGGG CCUUCUUG | 1989 |
| 559 | UACUUUCU G UGGACUCA | 592 | UCACUCCA GGAGGAAACUCC CU UCAAGGACAUCGUCCGGG ACAAACUA | 1990 |
| 561 | CUUUCUGU G GACUGAGA | 798 | UCUCACUC GGAGGAAACUCC CU UCAAGGACAUCGUCCGGG ACACAAAC | 1991 |
| 562 | UUUCUGUG G ACUGAGAA | 799 | UUCUCAGU GGAGGAAACUCC CU UCAAGGACAUCGUCCGGG CACACAAA | 1992 |
| 566 | UGUGGACU G ACAACUUU | 514 | AAACUUCU GGAGGAAACUCC CU UCAAGGACAUCGUCCGGG ACUCCACA | 1993 |
| 568 | UCCACUGA G AAGUUUCC | 800 | CCAAACUU GGAGGAAACUCC CU UCAAGGACAUCGUCCGGG UCACUCCA | 1994 |
| 571 | ACUGAGAA G UUUCCAUC | 593 | CAUCCAAA GGAGGAAACUCC CU UCAAGGACAUCGUCCGGG UUCUCACU | 1995 |
| 589 | CUAAAUAA G CUCCUAGA | 594 | UCUACCAC GGAGGAAACUCC CU UCAAGGACAUCGUCCGGG UUAUUUAC | 1996 |
| 592 | AAUAAGCU G GUAGACUA | 801 | UAGUCUAC GGAGGAAACUCC CU UCAAGGACAUCGUCCGGG AGCUUAUU | 1997 |
| 593 | AUAAGCUG G UAGACUAC | 595 | GUAGUCUA GGAGGAAACUCC CU UCAAGGACAUCGUCCGGG CAGCUUAU | 1998 |
| 596 | AGCUGGUA G ACUACUAC | 802 | GUAGUAGU GGAGGAAACUCC CU UCAAGGACAUCGUCCGGG UACCAGCU | 1999 |
| 606 | CUACUACA G GACAAAUU | 803 | AAUUUGUC GGAGGAAACUCC CU UCAAGGACAUCGUCCGGG UGUAGUAG | 2000 |
| 607 | UACUACAG G ACAAAUUC | 804 | GAAUUUGU GGAGGAAACUCC CU UCAAGGACAUCGUCCGGG CUGUAGUA | 2001 |
| 624 | CAUCUCCA G ACAGAAGC | 805 | GCUUCUGU GGAGGAAACUCC CU UCAAGGACAUCGUCCGGG UGGAGAUG | 2002 |
| 628 | UCCAGACA G AAGCAGAU | 806 | AUCUGCUU GGAGGAAACUCC CU UCAAGGACAUCGUCCGGG UGUCUGGA | 2003 |
| 631 | AGACAGAA G CAGAUCUU | 596 | AAGAUCUG GGAGGAAACUCC CU UCAAGGACAUCGUCCGGG UUCUGUCU | 2004 |
| 634 | CAGAAGCA G AUCUUCCU | 807 | AGGAAGAU GGAGGAAACUCC CU UCAAGGACAUCGUCCGGG UGCUUCUG | 2005 |
| 645 | CUUCCUUA G AGACAGAA | 808 | UUCUGUCU GGAGGAAACUCC CU UCAAGGACAUCGUCCGGG UAAGGAAG | 2006 |
| 647 | UCCUUAGA G ACAGAACC | 809 | CGUUCUGU GGAGGAAACUCC CU UCAAGGACAUCGUCCGGG UCUAAGGA | 2007 |
| 651 | UAGAGACA G AACCCGAG | 810 | CUCCGGUU GGAGGAAACUCC CU UCAAGGACAUCGUCCGGG UGUCUCUA | 2008 |
| 657 | CAGAACCC G AGAAGACC | 515 | GGUCUUCU GGAGGAAACUCC CU UCAAGGACAUCGUCCGGG GGGUUCUG | 2009 |
| 659 | GAACCCCA G AAGACCAG | 911 | CUGGUCUU GGAGGAAACUCC CU UCAAGGACAUCGUCCGGG UCCGGUUC | 2010 |
| 662 | CCCGAGAA G ACCAGGGU | 812 | ACCCUGGU GGAGGAAACUCC CU UCAAGGACAUCGUCCGGG UUCUCGGG | 2011 |
| 667 | GAAGACCA G GGUCACCG | 813 | CGGUGACC GGAGGAAACUCC CU UCAAGGACAUCGUCCGGG UGCUCUUC | 2012 |
| 668 | AAGACCAG G GUCACCGG | 814 | CCCCUGAC GGAGGAAACUCC CU UCAAGGACAUCGUCCGGG CUCGUCUU | 2013 |
| 668 | AGACCAGG G UCACCCGG | 597 | CCCCGUGA GGAGGAAACUCC CU UCAAGGACAUCGUCCGGG CCUCGUCU | 2014 |
| 675 | GGGUCACC G GCGCAACA | 815 | UGUUGCCC GGAGGAAACUCC CU UCAAGGACAUCGUCCGGG GCUCACCC | 2015 |
| 676 | GGUCACCG G GCCAACAG | 816 | CUGUUCCC GGAGGAAACUCC CU UCAAGGACAUCGUCCGGG CGGUCACC | 2016 |
| 677 | GUCACCGG G GCAACAGC | 817 | GCUGUUCC GGAGGAAACUCC CU UCAAGGACAUCGUCCGGG CCCCUCAC | 2017 |
| 678 | UCACCGGG G CAACAGCC | 598 | GGCUCUUG GGAGGAAACUCC CU UCAAGGACAUCGUCCGGG CCCCCUGA | 2018 |
| 684 | GGGCAACA G CCUGGACC | 599 | GGUCCACG GGAGGAAACUCC CU UCAAGGACAUCGUCCGGG UGUUCCCC | 2019 |
| 688 | AACAGCCU G CACCCGAG | 818 | CUCCCCUC GGAGGAAACUCC CU UCAAGGACAUCGUCCGGG AGGCUCUU | 2020 |
| 689 | ACAGCCUG G ACCCCAGG | 819 | CCUCCCCU GGAGGAAACUCC CU UCAAGGACAUCGUCCGGG CAGCCUCU | 2021 |
| 693 | CCUGGACC G CAGGUCCC | 820 | CCCACCUC GGAGGAAACUCC CU UCAAGGACAUCGUCCGGG CGUCCAGG | 2022 |
| 694 | CUGGACCC G AGGUCCCA | 821 | UCCCACCU GGAGGAAACUCC CU UCAAGGACAUCGUCCGGG CCCUCCAG | 2023 |
| 696 | GGACCGCA G CUCCCAGG | 822 | CCUGGGAC GGAGGAAACUCC CU UCAAGGACAUCGUCCGGG UCCGGUCC | 2024 |
| 697 | CACCGGAG G UCCCAGCG | 600 | CCCUGGGA GGAGGAAACUCC CU UCAAGGACAUCGUCCGGG CUCCGGUC | 2025 |
| 703 | ACCUCCCA G GGAGCCCC | 823 | GGGCCUCC GGAGGAAACUCC CU UCAAGGACAUCGUCCGGG UGGGACCU | 2026 |
| 704 | CCUCCCAG G GACGCCCA | 924 | UGGGCCUC GGAGGAAACUCC CU UCAAGGACAUCGUCCGGG CUGGGACC | 2027 |
| 705 | GUCCCAGG G AGCCCCAC | 825 | GUGGGCCU GGAGGAAACUCC CU UCAAGGACAUCGUCCGGG CCUCCCAC | 2028 |
| 707 | CCCAGGCA G GCCCACAC | 826 | GUCUCCCC GGAGGAAACUCC CU UCAAGGACAUCGUCCGGG UCCCUGGC | 2029 |
| 708 | CCAGGGAG G CCCACACC | 601 | CCUCUGCG GGAGGAAACUCC CU UCAAGGACAUCGUCCGGG CUCCCUGC | 2030 |
| 720 | ACACCUCA G UGGGGCUG | 602 | CAGCCCCA GGAGGAAACUCC CU UCAAGGACAUCGUCCGGG UGAGGUGU | 2031 |
| 722 | ACCUCAUC G GGGCUGUC | 827 | CACAGCCC GGAGGAAACUCC CU UCAAGGACAUCGUCCGGG ACUGAGGU | 2032 |
| 723 | CCUCAGUG G GUCUGUGG | 828 | CCACAGCC GGAGGAAACUCC CU UCAAGGACAUCGUCCGGG CACUGAGG | 2033 |
| 724 | CUCAGUGG G GCUGUGGG | 829 | CCCACAGC GGAGGAAACUCC CU UCAAGGACAUCGUCCGGG CCACUGAG | 2034 |
| 725 | UCAGUGGG G CUGUGGGA | 603 | UCCCACAG GGAGGAAACUCC CU UCAAGGACAUCGUCCGGG CCCACUGA | 2035 |
| 728 | GUGGGGCU G UGGGACAA | 604 | UUCUCCCA GGAGGAAACUCC CU UCAAGGACAUCGUCCGGG AGCCCCAC | 2036 |
| 730 | GGGUCUGU G GGAGAAGA | 830 | UCUCUCCU GGAGGAAACUCC CU UCAAGGACAUCGUCCGGG ACAGCCCC | 2037 |
| 731 | GUGCUGUG G GAGAAGAA | 831 | UUCUUCUC GGAGGAAACUCC CU UCAAGGACAUCGUCCGGG CACAGCCC | 2038 |
| 732 | GGCUGUGG G AGAAGAAA | 832 | UUUCUUCU GGAGGAAACUCC CU UCAAGGACAUCGUCCGGG CCACAGCC | 2038 |
| 734 | CUGUGGGA G AAGAAAUC | 833 | GAUUUCUU GGAGGAAACUCC CU UCAAGGACAUCGUCCGGG UCCCACAG | 2040 |
| 737 | UGGGAGAA G AAAUCCGA | 834 | UCGGAUUU GGAGGAAACUCC CU UCAAGGACAUCGUCCGGG UUCUCCCA | 2041 |
| 744 | AGAAAUCC G ACCUUCGA | 516 | UCGAAGGU GGAGGAAACUCC CU UCAAGGACAUCGUCCGGG GGAUUUCU | 2042 |
| 751 | CGACCUUC G AUGAACCG | 517 | CGGUUCAU GGAGGAAACUCC CU UCAAGGACAUCGUCCGGG CAAGGUCG | 2043 |
| 754 | CCUUCGAU G AACCGGAA | 518 | UUCCGGUU GGAGGAAACUCC CU UCAAGGACAUCGUCCGGG AUCGAAGG | 2044 |
| 759 | GAUGAACC G GAAGCUGU | 835 | ACAGCUUC GGAGGAAACUCC CU UCAAGGACAUCGUCCGGG GGUUCAUC | 2045 |
| 760 | AUGAACCG G AAGCUGUC | 836 | GACAGCUU GGAGGAAACUCC CU UCAAGGACAUCGUCCGGG CGGUUCAU | 2046 |
| 763 | AACCGGAA G CUGUCGGA | 605 | UCCUACAG GGAGGAAACUCC CU UCAAGGACAUCGUCCGGG UUCCGGUU | 2047 |
| 766 | CGGAAGCU G UCGGAUCA | 606 | UGAUCCGA GGAGGAAACUCC CU UCAAGGACAUCGUCCGGG AGCUUCCG | 2048 |
| 769 | AAGCUGUC G GAUCACCC | 837 | GGGUGAUC GGAGGAAACUCC CU UCAAGGACAUCGUCCGGG GACAGCUU | 2048 |
| 770 | AGCUGUCG G AUCACCCC | 838 | GGGGUGAU GGAGGAAACUCC CU UCAAGGACAUCGUCCGGG CGACAGCU | 2050 |
| 781 | CACCCCCC G ACCCUUCC | 519 | GGAAGGGU GGAGGAAACUCC CU UCAAGGACAUCGUCCGGG GCGCGGUG | 2051 |
| 793 | CUUCCCCU G CACCACCA | 520 | UCCUCCUG GGAGGAAACUCC CU UCAAGGACAUCGUCCGGG AGCGGAAG | 2052 |
| 796 | CCCCUGCA G CACCACCA | 607 | UCCUCCUC GGAGGAAACUCC CU UCAAGGACAUCGUCCGGG UCCACCCC | 2053 |
| 799 | CUCCACCA G CACCACCA | 608 | UCCUCCUC GGAGGAAACUCC CU UCAAGGACAUCGUCCGGG UCCUCCAC | 2054 |
| 805 | CACCACCA G CACCACCC | 609 | CCCUCCUC GGAGGAAACUCC CU UCAAGGACAUCGUCCGGG UCCUCCUC | 2055 |
| 811 | CACCACCA G CCACACCC | 610 | CCCUCUCC GGAGGAAACUCC CU UCAAGGACAUCGUCCGGG UCCUCCUC | 2056 |
| 817 | CAGCCACA G CCUCCCCA | 611 | UCCCCACC GGAGGAAACUCC CU UCAAGGACAUCGUCCGGG UCUCCCUC | 2057 |
| 823 | CACCCUCC G CAAUAUGC | 521 | CCAUAUUC GGAGGAAACUCC CU UCAAGGACAUCGUCCGGG CCACCCUC | 2058 |
| 830 | CGCAAUAU G CCCCAGCC | 522 | CCCUCCCC GGAGGAAACUCC CU UCAAGGACAUCGUCCGGG AUAUUCCC | 2059 |
| 836 | AUCCCCCA G CCCCCCAC | 612 | CUCCCCCC GGAGGAAACUCC CU UCAAGGACAUCGUCCGGG UCCCCCAU | 2060 |
| 838 | CCCCCACC G CCCCACCA | 523 | UCCUCCCC GGAGGAAACUCC CU UCAAGGACAUCGUCCGGG CCUCCCCC | 2061 |
| 844 | GCCCCCCA G CAGCUGCA | 613 | UCCACCUC GGAGGAAACUCC CU UCAAGGACAUCGUCCGGG UCCCCCCC | 2062 |
| 847 | CCCCACCA G CUGCAGCA | 614 | UCCUCCAC GGAGGAAACUCC CU UCAAGGACAUCGUCCGGG UCCUCCCC | 2063 |
| 850 | CACCACCU G CAGCAGCC | 524 | CCCUCCUC GGAGGAAACUCC CU UCAAGGACAUCGUCCGGG ACCUCCUC | 2064 |
| 853 | CAGCUGCA G CAGCCCCC | 615 | GGGGGCUG GGAGGAAACUCC CU UCAAGGACAUCGUCCGGG UGCAGCUG | 2065 |
| 856 | CUGCAGCA G CCCCCACA | 616 | UGUOGGGG GGAGGAAACUCC CU UCAAGGACAUCGUCCGGG UGCUGCAG | 2066 |
| 865 | CCCCCACA G CAGCGAUA | 617 | UAUCGCUG GGAGGAAACUCC CU UCAAGGACAUCGUCCGGG UGUGGUGG | 2067 |
| 868 | CCACAGCA G CGAUAUCU | 618 | AGAUAUCG GGAGGAAACUCC CU UCAAGGACAUCGUCCGGG UGCUGUGG | 2068 |
| 870 | ACAGCAGC G AUAUCUGC | 525 | GCAGAUAU GGAGGAAACUCC CU UCAAGGACAUCGUCCGGG GCUGCUGU | 2069 |
| 877 | CGAUAUCU G CAGCACCA | 526 | UGGUGCUG GGAGGAAACUCC CU UCAAGGACAUCGUCCGGG AGAUAUCG | 2070 |
| 880 | UAUCUGCA G CACCACCA | 619 | UGGUGGUG GGAGGAAACUCC CU UCAAGGACAUCGUCCGGG UCCAGAUA | 2071 |
| 898 | UUCCACCA G GAACGCCG | 839 | CGGCGUUC GGAGGAAACUCC CU UCAAGGACAUCGUCCGGG UGGUGGAA | 2072 |
| 899 | UCCACCAC G AACGCCCA | 840 | UCGGCCUU GGAGGAAACUCC CU UCAAGGACAUCGUCCGGG CUGGUGGA | 2073 |
| 903 | CCAGGAAC G COGAGGAG | 527 | CUCCUCUC GGAGGAAACUCC CU UCAAGGACAUCGUCCGGG GUUCCUGC | 2074 |
| 906 | GGAACGCC G AGGACGCA | 528 | UCCCUCCU GGAGGAAACUCC CU UCAAGGACAUCGUCCGGG GGCGUUCC | 2075 |
| 908 | AACGCCGA G GAGGCAGC | 841 | GCUGCCUC GGAGGAAACUCC CU UCAAGGACAUCGUCCGGG UCGGCGUU | 2076 |
| 909 | ACGCCGAG G AGGCAGCC | 842 | GCCUGCCU GGAGGAAACUCC CU UCAAGGACAUCGUCCGGG CUCGGCGU | 2077 |
| 911 | CCCGAGGA G GCAGCCUU | 843 | AAGGCUGC GGAGGAAACUCC CU UCAAGGACAUCGUCCGGG UCCUCGGC | 2078 |
| 912 | CCGAGGAG G CAGOCCUG | 620 | CAAGGCUG GGAGGAAACUCC CU UCAAGGACAUCGUCCGGG CUCCUCGG | 2079 |
| 915 | AGGAGGCA G CCUUGACA | 621 | UGUCAAGG GGAGGAAACUCC CU UCAAGGACAUCGUCCGGG UCCCUCCU | 2080 |
| 920 | GCAGCCUU G ACAUAAAU | 529 | AUUUAUGU GGAGGAAACUCC CU UCAAGGACAUCGUCCGGG AAGCCUCC | 2081 |
| 929 | ACAUAAAU G AUGCGCAU | 530 | AUGCCCAU GGAGGAAACUCC CU UCAAGGACAUCGUCCGGG AUUUAUGU | 2082 |
| 932 | UAAAUGAU G CGCAUUGU | 844 | ACAAUGCC GGAGGAAACUCC CU UCAAGGACAUCGUCCGGG AUCAUUUA | 2083 |
| 933 | AAAUCAUG G GCAUUGUG | 845 | CACAAUGC GGAGGAAACUCC CU UCAAGGACAUCGUCCGGG CAUCAUUU | 2084 |
| 934 | AAUGAUGG G CAUUCUGG | 622 | CCACAAUG GGAGGAAACUCC CU UCAAGGACAUCGUCCGGG CCAUCAUU | 2085 |
| 939 | UGGGCAUU G UGGCACCG | 623 | CGGUGCCA GGAGGAAACUCC CU UCAAGGACAUCGUCCGGG AAUCCCCA | 2086 |
| 941 | GGCAUUGU G CCACCGGC | 846 | GCCCGUGC GGAGGAAACUCC CU UCAAGGACAUCGUCCGGG ACAAUGCC | 2087 |
| 942 | GCAUUCUC G CACCCGCU | 624 | AGCCGCUG GGAGGAAACUCC CU UCAAGGACAUCGUCCGGG CACAAUGC | 2088 |
| 947 | GUGOCACO G GCUUGGGC | 847 | GCCCAAGC GGAGGAAACUCC CU UCAAGGACAUCGUCCGGG GGUGCCAC | 2089 |
| 948 | UGGCACCG G CUUGGGCA | 625 | UGCCCAAG GGAGGAAACUCC CU UCAAGGACAUCGUCCGGG CGGUGCCA | 2090 |
| 952 | ACCCGCUU G GGCAGUGA | 848 | UCACUGCC GGAGGAAACUCC CU UCAAGGACAUCGUCCGGG AAGCCGGU | 2091 |
| 953 | CCCCCUUC G CCAGUGAA | 849 | UUCACUGC GGAGGAAACUCC CU UCAAGGACAUCGUCCGGG CAACCCGG | 2092 |
| 954 | CCCCUUGC G CAGUGAAA | 626 | UUUCACUG GGAGGAAACUCC CU UCAAGGACAUCGUCCGGG CCAACCCG | 2093 |
| 957 | CUUGGGCA G UGAAAUGA | 627 | UCAUUUCA GGAGGAAACUCC CU UCAAGGACAUCGUCCGGG UCCCCAAG | 2094 |
| 959 | UCGGCAGU G AAAUCAAU | 531 | AUUCAUUU GGAGGAAACUCC CU UCAAGGACAUCGUCCGGG ACUGOCCA | 2095 |
| 964 | AGUGAAAU G AAUCCCCC | 532 | GCCGCAUU GGAGGAAACUCC CU UCAAGGACAUCGUCCGGG AUUUCACU | 2096 |
| 968 | AAAUCAAU G CCCCCCUC | 533 | GAGCCCCC GGAGGAAACUCC CU UCAAGGACAUCGUCCGGG AUUCAUUU | 2097 |
| 970 | AUCAAUGC G GCCCUCAU | 850 | AUGAGGGC GGAGGAAACUCC CU UCAAGGACAUCGUCCGGG CCAUUCAU | 2098 |
| 971 | UGAAUGCG G CCCUCAUC | 628 | CAUGAGGG GGAGGAAACUCC CU UCAAGGACAUCGUCCGGG CGCAUCCA | 2099 |
| 979 | GCCCUCAU G CAUCGGAG | 534 | CUCCGAUG GGAGGAAACUCC CU UCAAGGACAUCGUCCGGG AUCAGGGC | 2100 |
| 984 | CAUCCAUC G GAGACACA | 851 | UGUGUCUC GGAGGAAACUCC CU UCAAGGACAUCGUCCGGG GAUGGAUG | 2101 |
| 985 | AUGCAUCG G AGACACAC | 852 | GUGUGUOU GGAGGAAACUCC CU UCAAGGACAUCGUCCGGG CGAUGCAU | 2102 |
| 987 | GCAUCGGA G ACACACAG | 853 | CUGUGUGU GGAGGAAACUCC CU UCAAGGACAUCGUCCGGG UCCGAUGC | 2103 |
| 995 | GACACACA G ACCOAGUG | 854 | CACUGGGU GGAGGAAACUCC CU UCAAGGACAUCGUCCGGG UGUGUGUC | 2104 |
| 1001 | CAGACCCA G UGCAGCUC | 629 | GAGCUGCA GGAGGAAACUCC CU UCAAGGACAUCGUCCGGG UGGGUCUG | 2105 |
| 1003 | GACCCAGU G CAGCUCCA | 535 | UGGACCUG GGAGGAAACUCC CU UCAAGGACAUCGUCCGGG ACUGGGUC | 2106 |
| 1006 | CCAGUGGA G CUCCAGGC | 630 | GCCUGGAG GGAGGAAACUCC CU UCAAGGACAUCGUCCGGG UGCACUGG | 2107 |
| 1012 | CAGCUCCA G GCGGCAGG | 855 | CCUGCCGC GGAGGAAACUCC CU UCAAGGACAUCGUCCGGG UGGAGCUG | 2108 |
| 1013 | AGCUCCAG G CGGCAGGG | 631 | CCCUGCCG GGAGGAAACUCC CU UCAAGGACAUCGUCCGGG CUGGAGCU | 2109 |
| 1015 | CUCCAGGC G GCAGGGCG | 856 | CGCCCUGC GGAGGAAACUCC CU UCAAGGACAUCGUCCGGG GCCUGGAG | 2110 |
| 1016 | UCCAGGCG G CAGGGCGA | 632 | UCGCCCUG GGAGGAAACUCC CU UCAAGGACAUCGUCCGGG CGCCUGGA | 2111 |
| 1019 | AGGCGGCA G GGCGAGUG | 857 | CACUCGCC GGAGGAAACUCC CU UCAAGGACAUCGUCCGGG UGCCGCCU | 2112 |
| 1020 | GGCGGCAG G GCGAGUGC | 858 | GCACUCGC GGAGGAAACUCC CU UCAAGGACAUCGUCCGGG CUGCCGCC | 2113 |
| 1021 | GCGGCAGG G CGAGUGCG | 633 | CGCACUCG GGAGGAAACUCC CU UCAAGGACAUCGUCCGGG CCUGCCGC | 2114 |
| 1023 | GGCAGGGC G AGUGCGGU | 536 | ACCGCACU GGAGGAAACUCC CU UCAAGGACAUCGUCCGGG GCCCUGCC | 2115 |
| 1025 | CAGGGCGA G UGCCGUGG | 634 | CCACCGCA GGAGGAAACUCC CU UCAAGGACAUCGUCCGGG UCGCCCUG | 2116 |
| 1027 | GGGCCAGU G CGGUGGGC | 537 | GCCCACCG GGAGGAAACUCC CU UCAAGGACAUCGUCCGGG ACUCGCCC | 2117 |
| 1029 | GCGAGUGC G GUGGGCCC | 859 | GGGCCCAC GGAGGAAACUCC CU UCAAGGACAUCGUCCGGG GCACUCGC | 2118 |
| 1030 | CGACUCCG G UGGCCCCG | 635 | CGGCCCCA GGAGGAAACUCC CU UCAAGGACAUCGUCCGGG CGCACUCG | 2119 |
| 1032 | AGUGCGCU G GCCCCGGG | 860 | CCCGGCCC GGAGGAAACUCC CU UCAAGGACAUCGUCCGGG ACCCCACU | 2120 |
| 1033 | GUGCCCUG G GGCCCCGC | 861 | CCCCCCCC GGAGGAAACUCC CU UCAAGGACAUCGUCCGGG CACCGCAC | 2121 |
| 1034 | UGCCCUCG G CCCGGGCG | 636 | CCCCCGCC GGAGGAAACUCC CU UCAAGGACAUCGUCCGGG CCACCGCA | 2122 |
| 1038 | GUGGCCCC G CGCCCUGU | 862 | ACACCCCC GGAGGAAACUCC CU UCAAGGACAUCGUCCGGG GGGCCCAC | 2123 |
| 1039 | UGCCCCCC G GCCCUGUA | 863 | UACACCGC GGAGGAAACUCC CU UCAAGGACAUCGUCCGGG CGGGCCCA | 2124 |
| 1040 | GGCCCCGG G CGCUGUAU | 637 | AUACACCC GGAGGAAACUCC CU UCAAGGACAUCGUCCGGG CCGGGCCC | 2125 |
| 1042 | GCCCGGGC G CUGUAUGA | 538 | UCAUACAC GGAGGAAACUCC CU UCAAGGACAUCGUCCGGG GCCCGGGC | 2126 |
| 1045 | CGGGCGCU G UAUGACUU | 638 | AACUCAUA GGAGGAAACUCC CU UCAAGGACAUCGUCCGGG AGCGCCCG | 2127 |
| 1049 | CGCUGUAU G ACUUUGAG | 539 | CUCAAAGU GGAGGAAACUCC CU UCAAGGACAUCGUCCGGG AUACAGCG | 2128 |
| 1055 | AUGACUUU G AGGCCCUG | 540 | CACCCCCU GGAGGAAACUCC CU UCAAGGACAUCGUCCGGG AAAGUCAU | 2129 |
| 1057 | GACUUUGA G GCCCUGCA | 864 | UCCACCCC GGAGGAAACUCC CU UCAAGGACAUCGUCCGGG UCAAACUC | 2130 |
| 1058 | ACUUUGAG G CCCUGGAG | 639 | CUCCACCC GGAGGAAACUCC CU UCAAGGACAUCGUCCGGG CUCAAAGU | 2131 |
| 1063 | GAGGCCCU G GAGGAUGA | 865 | UCAUCCUC GGAGGAAACUCC CU UCAAGGACAUCGUCCGGG AGGGCCUC | 2132 |
| 1064 | AGGCCCUG G AGGAUGAC | 866 | GUCAUCCU GGAGGAAACUCC CU UCAAGGACAUCGUCCGGG CAGGGCCU | 2133 |
| 1066 | GCCCUGGA G GAUGACGA | 867 | UCCUCAUC GGAGGAAACUCC CU UCAAGGACAUCGUCCGGG UCCAGGGC | 2134 |
| 1067 | CCCUGGAG G AUGACCAG | 666 | CUCGUCAU GGAGGAAACUCC CU UCAAGGACAUCGUCCGGG CUCCAGGG | 2135 |
| 1070 | UGGAGGAU G ACGAGCUG | 541 | CAGCUCGU GGAGGAAACUCC CU UCAAGGACAUCGUCCGGG AUCCUCCA | 2136 |
| 1073 | AGGAUGAC G AGCUGGGG | 542 | CCCCAGCU GGAGGAAACUCC CU UCAAGGACAUCGUCCGGG GUCAUCCU | 2137 |
| 1075 | GAUGACGA G CUGGGGUU | 640 | AACCCCAG GGAGGAAACUCC CU UCAAGGACAUCGUCCGGG UCGUCAUC | 2138 |
| 1078 | GACGAGCU G GGGUUCCA | 869 | UGGAACCC GGAGGAAACUCC CU UCAAGGACAUCGUCCGGG AGCUCGUC | 2139 |
| 1079 | ACGAGCUG G GGUUCCAC | 870 | GUGGAACC GGAGGAAACUCC CU UCAAGGACAUCGUCCGGG CAGCUCGU | 2140 |
| 1080 | CGAGCUGG G GOUCCACA | 871 | UGUGGAAC GGAGGAAACUCC CU UCAAGGACAUCGUCCGGG CCAGCUCG | 2141 |
| 1081 | GACCUGGG G UUCCACAG | 641 | CUGUGGAA GGAGGAAACUCC CU UCAAGGACAUCGUCCGGG CCCAGCUC | 2142 |
| 1069 | GUUCCACA G CGGGGAGG | 642 | CCUCCCCG GGAGGAAACUCC CU UCAAGGACAUCGUCCGGG UGUGGAAC | 2143 |
| 1091 | UCCACAGC G GCGAGGUG | 872 | CACCUCCC GGAGGAAACUCC CU UCAAGGACAUCGUCCGGG GCUCUGGA | 2144 |
| 1092 | CCACAGCC G GGAGGUGG | 873 | CCACCUCC GGAGGAAACUCC CU UCAAGGACAUCGUCCGGG CGCUGUCC | 2145 |
| 1093 | CACAGCGG G GAGGUGGU | 874 | ACCACCUC GGAGGAAACUCC CU UCAAGGACAUCGUCCGGG CCGCUGUC | 2146 |
| 1094 | ACACCGGG G ACCUGGUG | 875 | CACCACCU GGAGGAAACUCC CU UCAAGGACAUCGUCCGGG CCCGCUCU | 2147 |
| 1096 | ACCGGGGA G GUGGUGGA | 876 | UCCACCAC GGAGGAAACUCC CU UCAAGGACAUCGUCCGGG UCCCCGCU | 2148 |
| 1097 | GCCCCCAG G UGGUGGAG | 643 | CUCCACCA GGAGGAAACUCC CU UCAAGGACAUCGUCCGGG CUCCCCGC | 2149 |
| 1099 | GGGCAGGU G GUGGAGGU | 877 | ACCUCCAC GGAGGAAACUCC CU UCAAGGACAUCGUCCGGG ACCUCCCC | 2150 |
| 1100 | GGGAGGUG G UGGAGGUC | 644 | GACCUCCA GGAGGAAACUCC CU UCAAGGACAUCGUCCGGG CACCUCCC | 2151 |
| 1102 | GAGGUGGU G GAGGUCCU | 878 | AGGACCUC GGAGGAAACUCC CU UCAAGGACAUCGUCCGGG ACCACCUC | 2152 |
| 1103 | AGGUGGUC G AGGUCCUG | 879 | CAGGACCU GGAGGAAACUCC CU UCAAGGACAUCGUCCGGG CACCACCU | 2153 |
| 1105 | GUGGUGGA G GUCCUGGA | 880 | UCCAGGAC GGAGGAAACUCC CU UCAAGGACAUCGUCCGGG UCCACCAC | 2154 |
| 1106 | UGGUGGAG G UCCUGGAU | 645 | AUCCAGGA GGAGGAAACUCC CU UCAAGGACAUCGUCCGGG CUCCACCA | 2155 |
| 1111 | GAGGUCCU G GAUAGCUC | 881 | CAGCUAUC GGAGGAAACUCC CU UCAAGGACAUCGUCCGGG ACCACCUC | 2156 |
| 1112 | AGGUCCUC G AUAGCUCC | 882 | CGACCUAU GGAGGAAACUCC CU UCAAGGACAUCGUCCGGG CAGGACCU | 2157 |
| 1116 | CCUGGAUA G CUCCAACC | 646 | CGUUCCAG GGAGGAAACUCC CU UCAAGGACAUCGUCCGGG UAUCCACC | 2158 |
| 1131 | CCCAUCCU G GUGGACCG | 883 | CCCUCCAC GGAGGAAACUCC CU UCAAGGACAUCGUCCGGG AGCAUGGC | 2159 |
| 1132 | CCAUCCUG G UCGACCCG | 647 | CCCCUCCA GGAGGAAACUCC CU UCAAGGACAUCGUCCGGG CACCAUCC | 2160 |
| 1134 | AUCCUCGU G GACCGGCC | 884 | CCCCCCUC GGAGGAAACUCC CU UCAAGGACAUCGUCCGGG ACCACCAU | 2161 |
| 1135 | UCCUGGUG G ACCCCCCG | 885 | CGGCCCCU GGAGGAAACUCC CU UCAAGGACAUCGUCCGGG CACCACCA | 2162 |
| 1139 | GCUGCACC G CCCCCCUC | 886 | CACCCCCC GGAGGAAACUCC CU UCAAGGACAUCGUCCGGG CCUCCACC | 2163 |
| 1140 | GUGGACCC G CCGCCUGC | 648 | CCACGCCG GGAGGAAACUCC CU UCAAGGACAUCGUCCGGG CCCUCCAC | 2164 |
| 1143 | GACCGGCC G CCUGCACA | 543 | UCUCCAGG GGAGGAAACUCC CU UCAAGGACAUCGUCCGGG CCCCCCUC | 2165 |
| 1147 | GCCCGCCU G CACAACAA | 544 | UUCUUGUC GGAGGAAACUCC CU UCAAGGACAUCGUCCGGG ACCCGCCC | 2166 |
| 1156 | CACAACAA G CUGGGCCU | 649 | AGGCCCAG GGAGGAAACUCC CU UCAAGGACAUCGUCCGGG UUGUUGUG | 2167 |
| 1159 | AACAAGCU G GGCCUCUU | 887 | AAGAGGCC GGAGGAAACUCC CU UCAAGGACAUCGUCCGGG AGCUUGUU | 2168 |
| 1160 | ACAAGCUG G GCCUCUUC | 888 | GAAGAGGC GGAGGAAACUCC CU UCAAGGACAUCGUCCGGG CAGCUUGU | 2169 |
| 1161 | CAAGCUGG G ocucuwec | 650 | GGAAGAGG GGAGGAAACUCC CU UCAAGGACAUCGUCCGGG CCAGCUUG | 2170 |
| 1172 | UCUUCCCU G CCAACUAC | 545 | GUAGUUGG GGAGGAAACUCC CU UCAAGGACAUCGUCCGGG AGGGAAGA | 2171 |
| 1181 | CCAACUAC G UGGCACCC | 651 | GGGUGCCA GGAGGAAACUCC CU UCAAGGACAUCGUCCGGG GUAGUUGG | 2172 |
| 1183 | AACUACGU G GCACCCAU | 889 | AUGGGUGC GGAGGAAACUCC CU UCAAGGACAUCGUCCGGG ACGUAGUU | 2173 |
| 1184 | ACUACGUG G CACCCAUG | 652 | CAUGGGUG GGAGGAAACUCC CU UCAAGGACAUCGUCCGGG CACGUAGU | 2174 |
| 1192 | GCACCCAU G ACCCGAUA | 546 | UAUCGGGU GGAGGAAACUCC CU UCAAGGACAUCGUCCGGG AUGGGUGC | 2175 |
| 1197 | CAUGACCC G AUAAACUC | 547 | GAGUUUAU GGAGGAAACUCC CU UCAAGGACAUCGUCCGGG GOGUCAUG | 2176 |
| 1210 | ACUCUUCA G GOGACAGA | 890 | UCUGUCCC GGAGGAAACUCC CU UCAAGGACAUCGUCCGGG UGAAGAGU | 2177 |
| 1211 | CUCUUCAG G GGACAGAA | 891 | UUCUGUCC GGAGGAAACUCC CU UCAAGGACAUCGUCCGGG CUGAAGAG | 2178 |
| 1212 | UCUUCAGG G GACAGAAG | 892 | CUUCUGUC GGAGGAAACUCC CU UCAAGGACAUCGUCCGGG CCUGAAGA | 2178 |
| 1213 | CUUCAGGG G ACAGAAGC | 893 | GCUUCUGU GGAGGAAACUCC CU UCAAGGACAUCGUCCGGG CCCUGAAG | 2180 |
| 1217 | AGGGGACA G AAGCUUUU | 894 | AAAAGCUU GGAGGAAACUCC CU UCAAGGACAUCGUCCGGG UGUCCCCU | 2181 |
| 1220 | GGACAGAA G CUUUUUGU | 653 | ACAAAAAG GGAGGAAACUCC CU UCAAGGACAUCGUCCGGG UUCUGUCC | 2182 |
| 1227 | AGCUUUUU G UCUGGAGC | 654 | GCUCCAGA GGAGGAAACUCC CU UCAAGGACAUCGUCCGGG AAAAAGCU | 2183 |
| 1231 | UUUUGUCU G GAGCUGCC | 895 | GGCAGCUC GGAGGAAACUCC CU UCAAGGACAUCGUCCGGG AGACAAAA | 2184 |
| 1232 | UUUGUCUG G AGCUGCCC | 896 | GGGCAGCU GGAGGAAACUCC CU UCAAGGACAUCGUCCGGG CAGACAAA | 2185 |
| 1234 | UGUCUGGA G CUGCCCAC | 655 | GUGGGCAG GGAGGAAACUCC CU UCAAGGACAUCGUCCGGG UCCAGACA | 2186 |
| 1237 | CUGGAGCU G CCCACAAG | 548 | CUUGUGGG GGAGGAAACUCC CU UCAAGGACAUCGUCCGGG AGCUCCAG | 2187 |
| 1245 | GCCCACAA G AAAGAGGG | 897 | CCCUCUUU GGAGGAAACUCC CU UCAAGGACAUCGUCCGGG UUGUGGGC | 2188 |
| 1249 | ACAAGAAA G AGGGCAAG | 898 | CUUGCCCU GGAGGAAACUCC CU UCAAGGACAUCGUCCGGG UUUCUUGU | 2189 |
| 1251 | AAGAAAGA G GGCAAGGA | 899 | UCCUUGCC GGAGGAAACUCC CU UCAAGGACAUCGUCCGGG UCUUUCUU | 2190 |
| 1252 | AGAAAGAG G GCAAGGAA | 900 | UUCCUUGC GGAGGAAACUCC CU UCAAGGACAUCGUCCGGG CUCUUUCU | 2191 |
| 1253 | GAAAGAGG G CAAGGAAA | 656 | UUUCCUUG GGAGGAAACUCC CU UCAAGGACAUCGUCCGGG CCUCUUUC | 2192 |
| 1257 | GAGGGCAA G GAAAAAAG | 901 | CUUUUUUC GGAGGAAACUCC CU UCAAGGACAUCGUCCGGG UUGCCCUC | 2193 |
| 1258 | AGGGCAAC G AAAAAAGG | 902 | CCUUUUUU GGAGGAAACUCC CU UCAAGGACAUCGUCCGGG CUUCCCCU | 2194 |
| 1265 | GGAAAAAA G GCUGGACU | 903 | AGUCCAGC GGAGGAAACUCC CU UCAAGGACAUCGUCCGGG UUUUUUCC | 2195 |
| 1266 | GAAAAAAG G CUGGACUC | 657 | GAGUCCAG GGAGGAAACUCC CU UCAAGGACAUCGUCCGGG CUUUUUUC | 2196 |
| 1269 | AAAAGCCU G GACUCCAU | 904 | AUGGAGUC GGAGGAAACUCC CU UCAAGGACAUCGUCCGGG ACCCUUUU | 2197 |
| 1270 | AAAGGCUG G ACUCCAUC | 905 | CAUGCACU GGAGGAAACUCC CU UCAAGGACAUCGUCCGGG CAGCCUUU | 2198 |
| 1278 | CACUCCAU G ACUAUAUA | 549 | UAUAUACU GGAGGAAACUCC CU UCAAGGACAUCGUCCGGG AUGGACUC | 2199 |

Input Sequence = HSA011736.
Cut Site = G/.
Stem Length = 8.
Core Sequence = GGAGGAAACCCC CU UCAAGGACAUCGUCCGGG
HSA011736 (Homo sapiens mRNA for growth factor receptor binding protein (GRGLG); 1303 bp)
TABLE IX

Human GRID GeneBloc and Substrate Sequence

| | | | | | |
|---|---|---|---|---|---|
| Pos | Substrate | Seq ID | GeneBloc | GB Seq ID | GB RPI# |

| | | | | | |
|---|---|---|---|---|---|
| 324 | GAGGAGUGGUUUAAGGCGGAGCU | 2201 | aₛgₛcₛuccgCₛCₛTₛTₛAₛAₛAₛCₛCₛacucₛcₛuₛc B | 2212 | 14006 |
| 445 | UCAUGGGCAAGGAGGUUGGCUUC | 2201 | gₛaₛaₛgccaAₛCₛCₛTₛCₛCₛTₛTₛGₛcccaₛuₛgₛa B | 2213 | 14007 |
| 456 | GAGGUUGGCUUCUUCAUCAUCCG | 2202 | cₛgₛgₛaugaTₛGₛAₛAₛGₛAₛAₛGₛCₛcaacₛcₛuₛc B | 2214 | 14008 |
| 736 | ACCUCAGUGGGGCUGUGGGAGAA | 2203 | uₛuₛcₛucccAₛCₛAₛGₛCₛCₛCₛCₛAₛcugaₛgₛgₛu B | 2215 | 14009 |
| 819 | CAGCACCAGCACCAGCCACAGCC | 2204 | gₛgₛcₛugugGₛCₛTₛGₛGₛTₛGₛCₛTₛggugₛcₛuₛg B | 2216 | 14010 |
| 825 | CAGCACCAGCCACAGCCUCCGCA | 2205 | uₛgₛcₛggagGₛCₛTₛGₛTₛGₛGₛCₛTₛggugₛcₛuₛg B | 2217 | 14011 |
| 951 | GAUGGGCAUUGUGGCACCGGCUU | 2206 | aₛaₛgₛccggTₛGₛCₛCₛAₛCₛAₛAₛTₛgcccₛaₛuₛc B | 2218 | 14012 |
| 1108 | ACAGCGGGGAGGUGGUGGAGGUC | 2207 | gₛcₛuₛauccAₛCₛCₛAₛCₛCₛTₛCₛCₛccgcₛuₛgₛu B | 2219 | 14013 |
| 1117 | AGGUGGUGGAGGUCCUGGAUAGC | 2208 | gₛaₛcₛcuccAₛGₛGₛAₛCₛCₛTₛCₛCₛaccaₛcₛcₛu B | 2220 | 14014 |
| 1162 | GCCGCCUGCACAACAAGCUGGGC | 2209 | gₛcₛcₛcagcTₛTₛGₛTₛTₛGₛTₛGₛCₛaggcₛgₛgₛc B | 2221 | 14015 |
| 1166 | CCUGCACAACAAGCUGGGCCUCU | 2210 | aₛgₛaₛggccCₛCₛCₛAₛGₛCₛTₛTₛGₛuuguₛgₛcₛa B | 2222 | 14016 |
| 1168 | UGCACAACAAGCUGGGCCUCUUC | 2211 | gₛaₛaₛgaggCₛCₛCₛAₛGₛCₛTₛTₛGₛuuguₛgₛcₛa B | 2223 | 14017 |
| 324 | GAGGAGUGGUUUAAGGCGGAGCU | 2200 | B agcuccgCₛCₛTₛTₛAₛAₛAₛCₛCₛacuccuc B | 2224 | 14540 |
| 445 | UCAUGGGCAAGGAGGUUGGCUUC | 2201 | B gaagccaAₛCₛCₛTₛCₛCₛTₛTₛGₛcccauga B | 2225 | 14541 |
| 456 | GAGGUUGGCUUCUUCAUCAUCCG | 2202 | B cggaugaTₛGₛAₛAₛGₛAₛAₛGₛCₛcaaccuc B | 2226 | 14542 |
| 736 | ACCUCAGUGGGGCUGUGGGAGAA | 2203 | B uucucccAₛCₛAₛGₛCₛCₛCₛCₛAₛcugaggu B | 2227 | 14543 |
| 819 | CAGCACCAGCACCAGCCACAGCC | 2204 | B ggcugugGₛCₛTₛGₛGₛTₛGₛCₛTₛggugcug B | 2228 | 14544 |
| 825 | CAGCACCAGCCACAGCCUCCGCA | 2205 | B ugcggagGₛCₛTₛGₛTₛGₛGₛCₛTₛggugcug B | 2229 | 14545 |
| 951 | GAUGGGGAUUGUGGCACCGGCUU | 2206 | B aagccggTₛGₛCₛCₛAₛCₛAₛAₛTₛgcccauc B | 2230 | 14546 |
| 11O8 | ACAGCGGGGAGGUGGUGGAGGUC | 2207 | B gaccuccAₛCₛCₛAₛCₛCₛTₛCₛCₛccgcugu B | 2231 | 14547 |
| 1117 | AGGUGGUGGAGGUCCUGGAUAGC | 2208 | B gcuauccAₛGₛGₛAₛCₛCₛTₛCₛCₛaccaccu B | 2232 | 14548 |
| 1162 | GCCGCCUGCACAACAAGGUGGGC | 2209 | B gcccagcTₛTₛGₛTₛTₛGₛTₛGₛCₛaggcggc B | 2233 | 14549 |
| 1166 | CCUGCACAACAAGCUGGGCCUCU | 2210 | B agaggccCₛAₛGₛCₛTₛTₛGₛTₛTₛgugcagg B | 2234 | 14550 |
| 1168 | UGCACAACAAGCUGGGCCUCUUC | 2211 | B gaagaggCₛCₛCₛAₛGₛCₛTₛTₛGₛuugugca B | 2235 | 14551 |

Upper Case = Ribo
Lower Case = 2′-O-Methyl
s = phosphorothioate linkage
B = inverted deoxyabasic ribonucleotide
Input Sequence = HSA011736
GB Length = 23
HSA011736 (Homo sapiens mRNA for growth factor receptor binding protein (GRBLG); 1303 bp)

TABLE X
Human Grid Enzymatic nucleic acid and target sequence

| | | | | | | |
|---|---|---|---|---|---|---|
| ref | | | | | Seq | |
| pos | Target | Seq ID | RPI# | Enzymatic Nucleic Acid | ID | Motif |

| | | | | | | |
|---|---|---|---|---|---|---|
| 13 | GCACAGU U AAUGGAU | 2256 | 23891 | auccauu CUGAUGAggccguuaggccGAA Acugugc B | 2280 | Hammerhead |
| 178 | ACUCAAU C UCUUCUC | 2257 | 23892 | Gagaaga CUGAUGAggccguuaggccCAA Auugagu B | 2281 | Hammerhead |
| 183 | AUCUCUU C UCUUCCA | 2258 | 23901 | uggaaga CUGAUGAggccguuaggccGAA Aagagau B | 2282 | Hammerhead |
| 317 | AGUGGUU U AAGGCGG | 2259 | 23902 | ccgccuu CUGAUGAggccguuaggccGAA Aaccacu B | 2283 | Hammerhead |
| 401 | CCUCUCU C GACACCA | 2260 | 23910 | ugguguc CUGAUGAggccguuaggccGAA Agagagg B | 2284 | Hammerhead |
| 179 | CUCAAUC U CUUCUCU | 2261 | 23911 | agagaag CUGAUGAggccguuaggccGAA Iauugag B | 2285 | Inozyme |
| 395 | CGAAGGC C UCUCUCG | 2262 | 23920 | cgagaga CUGAUGAggccguuaggcccAA Iccuucg B | 2286 | Inozyme |
| 412 | ACCAGGC A GAGAACU | 2263 | 23921 | aguucuc CUGAUGAggccguuaggccGAA Iccuggu B | 2287 | Inozyme |
| 638 | GCAGAUC U UCCUUAG | 2284 | 23929 | cuaagga CUGAUGAggccguuaggcccAA Iaucugc B | 2288 | Inozyme |
| 1268 | AAAAGGC U GGACUCC | 2265 | 23930 | ggagucc CUGAUGAggccguuaggccGAA Iccuuuu B | 2289 | Inozyme |
| 11 | AGGCACA G UUAAUGG | 2288 | 23890 | ccauuaa gccgaaaggCgaqugaGGuCu ugugccu B | 2290 | Zinzyme |
| 153 | ACUCGGU G UCAAAGC | 2267 | 23900 | gcuuuga gccgaaaggCgagugaGGuCu accgagu B | 2291 | Zinzyme |
| 370 | ACAUCCA G UUUCCCA | 2260 | 23909 | ugggaaa gccgaaaggCgagugaGGuCu uggaugu B | 2292 | Zinzyme |
| 817 | AGCCACA G CCUCCGC | 2269 | 23919 | gcggagg gccgaaaggCgagugaGGuCu uguggcu B | 2293 | Zinzyme |
| 880 | AUCUGCA G CACCACC | 2270 | 23928 | gguggug gccgaaaggCgagugaGGuCu ugcagau B | 2294 | Zinzyme |
| 409 | GACACCA G GCAGAGA | 2271 | 23893 | ucucugc CgaggaaacucC CUUCaaggacaucgucCGGG ugguguc B | 2295 | Amberzyme |
| 413 | CCAGGCA G AGAACUU | 2272 | 23903 | aaguucu GgaggaaacucC CUUCaaggacaucgucCGGG ugccugg B | 2296 | Amherzyme |
| 628 | CCAGACA G AAGCAGA | 2273 | 23912 | ucugcuu GgaggaaacucC CUUCaaggacaucgucCGGG ugucugg B | 2297 | Amberzyme |
| 1070 | GGAGGAU G ACGAGCU | 2274 | 23922 | agcucgu GgaggaaacucC CUUCaaggacaucgucCGGG auccucc B | 2298 | Amberzyme |
| 110 | AGGUGGU G GAGGUCC | 2275 | 23931 | ggaccuc GgaggaaacucC CUUCaaggacaucgucCGGG accaccu B | 2299 | Amberzyme |
| 8 | AGGAGGU A CAGUUAA | 2276 | 23889 | uuaacug GGCTAGCTACAACGA gccuccu B | 2300 | DNAzyme |
| 102 | AACUCUG A UGCUUGA | 2277 | 23899 | ucaagca GGCTAGCTACAACGA cagaguu B | 2301 | DNAzyme |
| 176 | AAACUCA A UCUCUUC | 2278 | 23908 | gaagaga GGCTAGCTACAACGA ugaguuu B | 2302 | DNAzyme |
| 370 | ACAUCCA G UUUCCCA | 2268 | 23918 | ugggaaa GGCTAGCTACAACGA uggaugu B | 2303 | DNAzyme |
| 403 | UCUCUCG A CACCAGG | 2279 | 23927 | cauggug GGCTAGCTACAACGA cgagaga B | 2304 | DNAzyme |

Lowercase = 2′-O-methyl nucleotide
Uppercase = ribonucleotide
B = inverted deoxyabasic moiety

What is claimed is:

1. A nucleic acid molecule which down regulates expression of a Grb2-related with Insert Domain (GRID) gene.

2. The nucleic acid molecule of claim 1, wherein said nucleic acid molecule is used to treat conditions selected from the group consisting of tissue/graft rejection and leukemia.

3. The nucleic acid molecule of claim 1, wherein said nucleic acid molecule is an enzymatic nucleic acid molecule having at least one binding arm.

4. The nucleic acid molecule of claim 3, wherein one or more binding arms of the enzymatic nucleic acid molecule comprises a sequence complementary to a sequence selected from the group consisting of SEQ ID NOS. 1-905 and 2256-2279.

5. The nucleic acid molecule of claim 3, wherein the enzymatic nucleic acid molecule comprises a sequence selected from the group consisting of SEQ ID NOS. 906-2199 and 2280-2304.

6. The nucleic acid molecule of claim 1, wherein said nucleic acid molecule is an antisense nucleic acid molecule.

7. The nucleic acid molecule of claim 6, wherein said antisense nucleic acid molecule comprises a sequence complementary to a sequence selected from the group consisting of SEQ ID NOS. 1-905, 2200-2211, and 2256-2279.

8. The nucleic acid molecule of claim 6, wherein said antisense nucleic acid molecule comprises a sequence selected from the group consisting of SEQ ID NOS. 2212-2235.

9. The nucleic acid molecule of claim 3, wherein said enzymatic nucleic acid molecule is in a hammerhead (HH) motif.

10. The nucleic acid molecule of claim 3, wherein said enzymatic nucleic acid molecule is in a hairpin, hepatitis Delta virus, group I intron, VS nucleic acid, amberzyme, zinzyme or RNAse P nucleic acid motif.

11. The nucleic acid molecule of claim 3, wherein said enzymatic nucleic acid molecule is in an Inozyme motif.

12. The nucleic acid molecule of claim 3, wherein said enzymatic nucleic acid molecule is in a G-cleaver motif.

13. The nucleic acid molecule of claim 3, wherein said enzymatic nucleic acid molecule is a DNAzyme.

14. The nucleic acid molecule of claim 3, wherein said enzymatic nucleic acid molecule comprises between 12 and 100 bases complementary to the RNA of a GRID gene.

15. The nucleic acid molecule of claim 3, wherein said enzymatic nucleic acid molecule comprises between 14 and 24 bases complementary to the RNA of a GRID gene.

16. The nucleic acid molecule of claim 1, wherein said nucleic acid molecule is chemically synthesized.

17. The nucleic acid molecule of claim 1, wherein said nucleic acid molecule comprises at least one 2′-sugar modification.

18. The nucleic acid molecule of claim 1, wherein said nucleic acid molecule comprises at least one nucleic acid base modification.

19. The nucleic acid molecule of claim 1, wherein said nucleic acid molecule comprises at least one phosphate backbone modification.

20. A mammalian cell including the nucleic acid molecule of claim 1.

21. The mammalian cell of claim 20, wherein said mammalian cell is a human cell.

22. A method of reducing GRID activity in a cell comprising the step of contacting said cell with the nucleic acid molecule of claim 1 under conditions suitable for said reduction of GRID activity.

23. A method of treatment of a patient having a condition associated with the level of GRID, comprising contacting cells of said patient with the nucleic acid molecule of claim 1, under conditions suitable for said treatment.

24. The method of claim 23 further comprising the use of one or more therapies under conditions suitable for said treatment.

25. A method of cleaving RNA of a GRID gene comprising the step of contacting the nucleic acid molecule of claim 1 with said RNA under conditions suitable for the cleavage of said RNA.

26. The method of claim 25, wherein said cleavage is carried out in the presence of a divalent cation.

27. The method of claim 26, wherein said divalent cation is Mg²⁺.

28. The nucleic acid molecule of claim 1, wherein said nucleic acid molecule comprises a cap structure at the 5′-end, the 3′-end or both the 5′-end and the 3′-end.

29. The nucleic acid molecule of claim 9, wherein one or more binding arms of the hammerhead motif comprises a sequence complementary to a sequence selected from the group consisting of SEQ ID NOS. 1-179 and 2256-2260.

30. The nucleic acid molecule of claim 11, wherein one or more binding arms of the Inozyme motif comprises a sequence complementary to a sequence selected from the group consisting of SEQ ID NOS. 180-492 and 2261-2265.

31. The nucleic acid molecule of claim 12, wherein one or more binding arms of the G-cleaver motif comprises a sequence complementary to a sequence selected from the group consisting of SEQ ID NOS. 493-657.

32. An expression vector comprising a nucleic acid sequence encoding at least one nucleic acid molecules of claim 1 in a manner which allows expression of the nucleic acid molecule.

33. A mammalian cell including an expression vector of claim 32.

34. The mammalian cell of claim 33, wherein said mammalian cell is a human cell.

35. The expression vector of claim 32, wherein said nucleic acid molecule is an enzymatic nucleic acid molecule.

36. The expression vector of claim 32, wherein said expression vector further comprises a sequence for an antisense nucleic acid molecule complementary to the RNA of a GRID gene.

37. The expression vector of claim 32, wherein said expression vector comprises a sequence encoding two or more of said nucleic acid molecules, which may be the same or different.

38. The expression vector of claim 37, wherein said expression vector comprises a nucleic acid sequence encoding an antisense nucleic acid molecule complementary to the RNA of a GRID gene.

39. The expression vector of claim 37, wherein said expression vector comprises a nucleic acid sequence encoding an enzymatic nucleic acid molecule complementary to the RNA of a GRID gene.

40. A method for treatment of tissue/graft rejection comprising the step of administering to a patient the nucleic acid molecule of claim 1 under conditions suitable for said treatment.

41. A method for treatment of leukemia comprising the step of administering to a patient the nucleic acid molecule of claim 1 under conditions suitable for said treatment.

42. An enzymatic nucleic acid molecule which cleaves RNA derived from a GRID gene.

43. The enzymatic nucleic acid molecule of claim 42, wherein said enzymatic nucleic acid molecule is selected from the group consisting of Hammerhead, Hairpin, Inozyme, G-cleaver, DNAzyme, Amberzyme and Zinzyme.

44. The method of any of claims 40 or 41, wherein said method further comprises administering to said patient one or more other therapies.

45. The method of claim 44, wherein said other therapies are therapies selected from the group consisting of radiation, chemotherapy, and cyclosporin treatment.

46. The nucleic acid molecule of claim 7, wherein said nucleic acid molecule comprises at least five ribose residues, at least ten 2′-O-methyl modifications, and a 3′- end modification.

47. The nucleic acid molecule of claim 46, wherein said nucleic acid molecule further comprises a phosphorothioate core with a 3′ and a 5′-end modification.

48. The nucleic acid molecule of any of claims 46 and 47, wherein said 3′ and/or 5′-end modification is 3′-3′ inverted abasic moiety.

49. The nucleic acid molecule of claim 3, wherein said nucleic acid molecule comprises at least five ribose residues, at least ten 2′-O-methyl modifications, and a 3′- end modification.

50. The nucleic acid molecule of claim 49, wherein said nucleic acid molecule further comprises phosphorothioate linkages on at least three of the 5′ terminal nucleotides.

51. The nucleic acid molecule of claim 49, wherein said 3′- end modification is 3′-3′ inverted abasic moiety.

52. The enzymatic nucleic acid molecule of claim 13, wherein said DNAzyme comprises at least ten 2′-O-methyl modifications and a 3′-end modification.

53. The enzymatic nucleic acid molecule of claim 52, wherein said DNAzyme further comprises phosphorothioate linkages on at least three of the 5′ terminal nucleotides.

54. The enzymatic nucleic acid molecule of claim 52, wherein said 3′- end modification is 3′-3′ inverted abasic moiety.
